(19) United States
(12) Patent Application Publication
(10) Pub. No.: US 2011/0257163 A1
(43) Pub. Date: Oct. 20, 2011

(54) GAMMA SECRETASE MODULATORS

(75) Inventors: Anandan Palani, Bridgewater, NJ (US); Jun Qin, Edison, NJ (US); Xiaohong Zhu, Edison, NJ (US); Robert G. Aslanian, Rockaway, NJ (US); Mark D. McBriar, Clinton, NJ (US)

(73) Assignee: Schering Corporation

(21) Appl. No.: 12/671,782

(22) PCT Filed: Aug. 4, 2008
(86) PCT No.: PCT/US08/09368

§ 371 Date: Jun. 23, 2011

Related U.S. Application Data
(60) Provisional application No. 60/954,468, filed on Aug. 7, 2007.

(51) Int. Cl. A61K 31/541; A61K 31/4164; C07D 417/10; A61P 27/06; A61P 25/00; A61P 25/28; A61P 3/06; C07D 233/61; A61K 31/427

(52) U.S. Cl. 514/222.2; 548/343.5; 514/399; 548/214; 514/372; 544/3

(57) ABSTRACT

This invention provides novel compounds that are modulators of gamma secretase. The compounds have the formula Compounds of formula (I) include compounds of formulas (IA) and (IB). Also disclosed are methods of modulating gamma secretase activity and methods of treating Alzheimer's Disease using the compounds of formula (I).

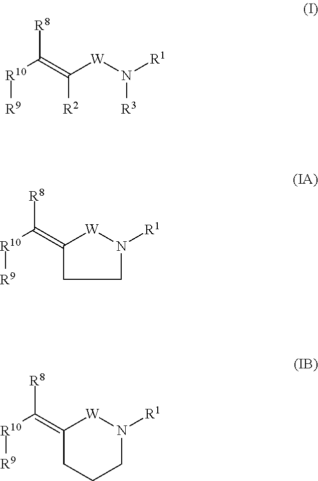

### REFERENCE TO RELATED APPLICATION

This application claims the benefit of U.S. Provisional Application Ser. No. 60/954,468 filed Aug. 7, 2007.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which is observed in familial Alzheimer's disease, increase production of Aβ40 and Aβ42 (for example, see Gouras G K, et al, Intraneuronal Aβ42 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272(51), p. 32247-32253.). Therefore, compounds which reduce production of Aβ40 and Aβ42 are expected as an agent for controlling progress of Alzheimer's disease or for preventing the disease.

These Aβs are produced when APP is cleaved by beta secretase and subsequently clipped by gamma secretase. In consideration of this, creation of inhibitors of γ secretase and β secretase has been attempted for the purpose of reducing production of Aβs. Many of these secretase inhibitors already known are peptides or peptidomimetics such as L-685,458. L-685,458, an aspartyl protease transition stale mimic, is a potent inhibitor of amyloid β-protein precursor γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics™, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

The compounds of this invention (Formula I) can be useful as gamma secretase modulators and can be useful in the treatment and prevention of diseases such as, for example, Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), Olfactory function loss (Getchell, et. al. Neurobiology of Aging, 663-673, 24, 2003).

This invention provides compounds of formula (A):

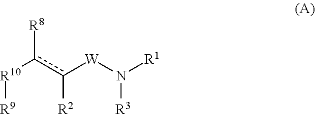

wherein the dotted line (----) represents an optional bond, and all substituents are as defined for formula (I) below. The embodiments of this invention include the embodiments described for formula (I) except that the embodiment is directed to a compound of formula (A). Thus, this invention includes compounds of formula (I) wherein there is a single bond, instead of a double bond, between the carbon to which R² is bound and the carbon to which R⁸ is bound. This invention also includes compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) and (IM) wherein there is a single bond, instead of a double bond, between the carbon to which R⁸ is bound and the ring carbon.

This invention also provides compounds of formula (I)

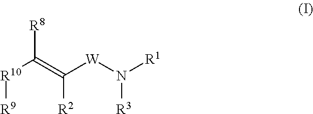

and the pharmaceutically acceptable salts, esters and solvates thereof, wherein W is —S(O)— or —S(O)₂, and R¹, R², R³, R⁸, R⁹, and R¹⁰ are as defined below.

This invention also provides a compound of formula (I) wherein R² and R³ can be taken together with the atoms to which they are bound to form a five or six membered heterocycloalkyl (heterocyclyl) ring, or a five or six membered heterocycloalkenyl (heterocyclenyl) ring. Each substitutable carbon atom of the ring is optionally substituted with one or two independently selected R²¹ groups. A substitutable nitrogen atom in the ring is optionally substituted with an R¹⁴ group. Examples of compounds of formula (I) wherein R² and R³ are taken together to form a ring include compounds of formulas (IA) to (IG).

This invention also provides a compound of formula (I).

This invention also provides a pharmaceutically acceptable salt of a compound of formula (I).

This invention also provides a pharmaceutically acceptable ester of a compound of formula (I).

This invention also provides a solvate of a compound of formula (I).

This invention also provides a compound of formula (I) in isolated form.

This invention also provides a compound of formula (I) in pure form.

This invention also provides a compound of formula (I) in pure and isolated form.

This invention also provides a compound of formula (I) selected from the group consisting of: (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) and (IM).

This invention also provides a compound selected from the group consisting of compounds 1.0 to 17.0.

This invention also provides a pharmaceutically acceptable salt of a compound selected from the group consisting of compounds 1.0 to 17.0.

This invention also provides a pharmaceutically acceptable ester of a compound selected from the group consisting of compounds 1.0 to 17.0.

This invention also provides a solvate of a compound selected from the group consisting of compounds 1.0 to 17.0.

This invention also provides a pharmaceutical composition comprising an effective amount of one or more (e.g., one) compounds of formula (I) and a pharmaceutically acceptable carrier.

This invention also provides a pharmaceutical composition comprising an effective amount of a pharmaceutically acceptable salt of one or more (e.g., one) compounds of formula (I) and a pharmaceutically acceptable carrier.

This invention also provides a pharmaceutical composition comprising an effective amount of a pharmaceutically acceptable ester of one or more (e.g., one) compounds of formula (I) and a pharmaceutically acceptable carrier.

This invention also provides a pharmaceutical composition comprising an effective amount of a solvate of one or more (e.g., one) compounds of formula (I) and a pharmaceutically acceptable carrier.

This invention also provides a pharmaceutical composition comprising an effective amount of one or more (e.g., one) compounds of formula (I), or pharmaceutically acceptable salts, esters or solvates thereof, and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

This invention also provides a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

This invention also provides pharmaceutical compositions comprising a combination of an effective amount of one or more (e.g., one) compounds of formula (I), in combination with an effective amount of one or more compounds selected from the group consisting of cholinesterase inhibitors, Aβ antibody inhibitors, gamma secretase inhibitors and beta secretase inhibitors. The pharmaceutical compositions also comprise a pharmaceutically acceptable carrier.

The compounds of formula (I) can be useful as gamma secretase modulators and can be useful in the treatment and prevention of diseases such as, for example, central nervous system disorders such as Alzheimers disease and Downs Syndrome.

Thus, this invention provides a method for modulating (including inhibiting, antagonizing and the like) gamma-secretase comprising administering an effective amount of one or more (e.g., one) compounds of formula (I) to a patient in need of such treatment.

This invention also provides a method of treating one or more neurodegenerative diseases, comprising administering an effective amount of one or more (e.g., one) compounds of formula (I) to a patient in need of treatment.

This invention also provides a method of inhibiting the deposition of amyloid protein (e.g., amyloid beta protein) in, on or around neurological tissue (e.g., the brain), comprising administering an effective amount of one or more (e.g., one) compounds of formula (I) to a patient in need of treatment.

This invention also provides a method of treating Alzheimer's disease, comprising administering an effective amount of one or more (e.g., one) compounds of formula (I) to a patient in need of treatment.

This invention also provides a method of treating Alzheimer's disease, comprising administering an effective amount of one or more (e.g., one) compounds of formula (I), in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Alzheimer's disease, comprising administering an effective amount of one or more compounds (e.g., one) of formula (I), in combination with an effective amount of one or more compounds selected from the group consisting of Aβ antibody inhibitors, gamma secretase inhibitors and beta secretase inhibitors.

This invention also provides a method of treating Downs syndrome, comprising administering an effective amount of one or more compounds (e.g., one) of formula (I) to a patient in need of treatment.

This invention also provides a method of treating Downs syndrome, comprising administering an effective amount of one or more (e.g., one) compounds of formula I, in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides combinations comprising an effective amount of one or more compounds of formula (I), in combination with an effective amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

This invention also provides a method of treating mild cognitive impairment, comprising administering an effective amount of one or more (e.g., one) compounds of formula (I) to a patient in need of treatment.

This invention also provides a method of treating glaucoma, comprising administering an effective amount of one or more (e.g., one) compounds of formula (I) to a patient in need of treatment.

This invention also provides a method of treating cerebral amyloid angiopathy, comprising administering an effective amount of one or more (e.g., one) compounds of formula (I) to a patient in need of treatment.

This invention also provides a method of treating stroke, comprising administering an effective amount of one or more (e.g., one) compounds of formula (I) to a patient in need of treatment.

This invention also provides a method of treating dementia, comprising administering an effective amount of one or more (e.g., one) compounds of formula (I) to a patient in need of treatment.

This invention also provides a method of treating microgliosis, comprising administering an effective amount of one or more (e.g., one) compounds of formula (I) to a patient in need of treatment.

This invention also provides a method of treating brain inflammation, comprising administering an effective amount of one or more (e.g., one) compounds of formula (I) to a patient in need of treatment.

This invention also provides a method of treating olfactory function loss, comprising administering an effective amount of one or more (e.g., one) compounds of formula (I) to a patient in need of treatment.

This invention also provides any one of the methods disclosed above and below wherein the compound of formula (I) is selected from the group consisting of the compounds (1.0) to (17.0).

This invention also provides any one of the pharmaceutical compositions disclosed above and below wherein the compound is selected from the group consisting of the compounds (1.0) to (17.0).

Other embodiments of this invention are directed to any one of the embodiments above or below that are directed to formula (I), or the use of formula (I) (e.g. the embodiments directed to methods of treatment, pharmaceutical compositions and kits), wherein the compound is a compound of formula A instead of formula I.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

### DETAILED DESCRIPTION OF THE INVENTION

This invention provides compounds, that are modulators of gamma secretase activity, of formula (I)

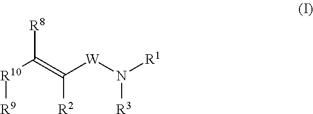

or the pharmaceutically acceptable salts, esters or solvates thereof, wherein:

R¹, R², R³, R⁸, R⁹, R¹⁰, and W are independently selected;

W is selected from the group consisting of; —S(O)—, and —S(O)₂—;

R¹ is selected from the group consisting of H, alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedheterocycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl- R¹ groups is optionally substituted with 1-5 independently selected R²¹ groups;

R² and R³ are each independently selected from the group consisting of H, alkyl-, alkenyl-, alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl-, and heterocyclyalkyl-; wherein each of said alkyl-, alkenyl- and alkynyl-, aryl-, arylalkyl-, alkylaryl-, cycloalkyl-, cycloalkenyl, cycloalkylalkyl-, cycloalkenyl-, heteroaryl-, heteroarylalkyl-, heterocyclyl-, heterocyclenyl- and heterocyclyalkyl- R¹ groups is optionally substituted with 1-5 independently selected R²¹ groups; or

R² and R³ taken together, along with the atoms to which they are bound, form a ring selected from the group consisting of:
(a) a 5 to 6 membered heterocycloalkyl ring, said heterocycloalkyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
   wherein said ring is optionally substituted with 1-5 independently selected R²¹ groups; and wherein:
(1) in one example the ring formed by R² and R³ being taken together comprises the heteroatom S (from the W moiety) and the heteroatom N adjacent to W;
(2) in another example the ring formed by R² and R³ being taken together comprises the heteroatom S (from the W moiety), the heteroatom N adjacent to W, and at least one (e.g., one) other heteroatom independently selected from the group consisting of: —O— and —NR¹⁴—; and
(3) in another example the ring formed by R² and R³ being taken together comprises the heteroatom S (from the W moiety), the heteroatom N adjacent to W, and the heteroatom —O—; and (4) in another example the ring formed by R² and R³ being taken together comprises the heteroatom S (from the W moiety), the heteroatom N adjacent to W, and the heteroatom —NR¹⁴—; or

R² and R³ taken together along with the atoms to which they are bound, and R¹ and R³ are taken together along with the atoms to which they are bound, form the fused ring moiety:

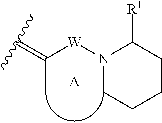

wherein Ring A represents the ring formed when R² and R³ are taken together to form a heterocycloalkyl ring, or a heterocycloalkenyl ring, as described above; that is Ring A is a ring selected from the group consisting of:
(a) a 5 to 6 membered heterocycloalkyl ring, said heterocycloalkyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
   wherein said fused ring moiety is optionally substituted with 1-5 independently selected R²¹ groups; or

R¹ and R³ taken together with the atoms to which they are bound form a fused benzoheterocycloalkyl (i.e., benzofusedheterocycloalkyl) ring, wherein said fused ring is optionally substituted with 1-5 independently selected R²¹ groups, and wherein in one example said fused ring is:

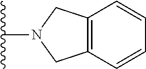

optionally substituted with 1-5 independently R²¹ groups (and in one example said fused ring is not substituted), and wherein in another example said fused ring is:

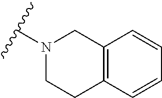

optionally substituted with 1-5 independently R²¹ groups (and in one example said fused ring is not substituted), and wherein in another example said fused ring is:

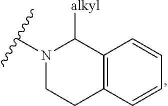

and wherein in another example said fused ring is:

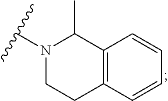

R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl-; wherein each of said R⁸ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl- is optionally substituted with 1-3 independently selected R²¹ groups;

R⁹ is selected from the group consisting of: alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, and heterocyclyalkyl-, wherein each of said R⁹ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl- and heterocyclyalkyl- is optionally substituted with 1-3 independently selected R²¹ groups;

R¹⁰ is selected from the group consisting of: a bond, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl-, heterocyclyalkenyl-,

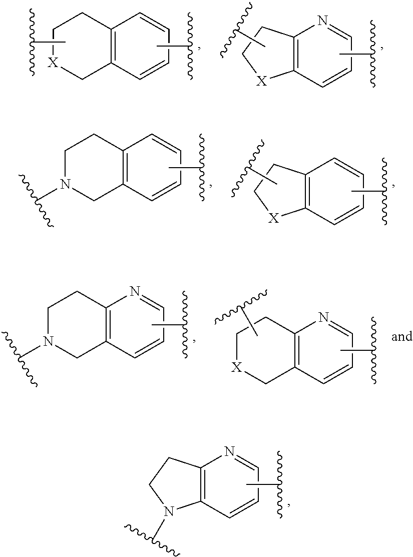

wherein X is selected from the group consisting of: O, —N(R¹⁴)— or —S—; and wherein each of said R¹⁰ moieties is optionally substituted with 1-3 independently selected R²¹ groups;

R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁸, and —P(O)(OR¹⁵)(OR¹⁶);

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;

Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

two R¹⁸ moieties on adjacent carbons can be linked together to form a

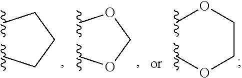

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

Each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR'⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups; and

Each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

Another embodiment of this invention is directed to compounds, that are modulators of gamma secretase activity, of formula (I)

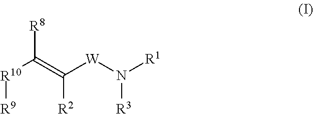

or the pharmaceutically acceptable salts, esters or solvates thereof, wherein:

W is selected from the group consisting of; —S(O)—, and —S(O)₂—;

R¹ is selected from the group consisting of H, alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedheterocycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl- R¹ groups is optionally substituted with 1-5 independently selected R²¹ groups;

R² and R³ are each independently selected from the group consisting of H, alkyl-, alkenyl-, alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl-, and heterocyclyalkyl-; wherein each of said alkyl-, alkenyl- and alkynyl-, aryl-, arylalkyl-, alkylaryl-, cycloalkyl-, cycloalkenyl, cycloalkylalkyl-, cycloalkenyl-, heteroaryl-, heteroarylalkyl-, heterocyclyl-, heterocyclenyl- and heterocyclyalkyl- R¹ groups is optionally substituted with 1-5 independently selected R²¹ groups; or

R² and R³ taken together, along with the atoms to which they are bound, form a 5 to 6 membered heterocycloalkyl ring, or a 5 to 6 membered heterocycloalkenyl ring, said heterocycloalkyl ring optionally comprising, in addition to W and in addition to the N adjacent to W, at least one (e.g., one) other heteroatom independently selected from the group consisting of: —O— and —NR¹⁴—, and said heterocycloalkenyl ring optionally comprising, in addition to W and in addition to the N adjacent to W, at least one (e.g., one) other heteroatom independently selected from the group consisting of: —O— and —NR¹⁴—; wherein (1) in one example the ring formed by R² and R³ being taken together comprises the heteroatom S (from the W moiety) and the heteroatom N adjacent to W; (2) in another example the ring formed by R² and R³ being taken together comprises the heteroatom S (from the W moiety), the heteroatom N adjacent to W, and at least one (e.g., one) other heteroatom independently selected from the group consisting of: —O— and —NR¹⁴—; (3) in another example the ring formed by R² and R³ being taken together comprises the heteroatom S (from the W moiety), the heteroatom N adjacent to W, and the heteroatom —O—; and (4) in another example the ring formed by R² and R³ being taken together comprises the heteroatom S (from the W moiety), the heteroatom N adjacent to W, and the heteroatom —NR¹⁴—;

R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl-; wherein each of said R⁸ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl- is optionally substituted with 1-3 independently selected R²¹ groups;

R⁹ is selected from the group consisting of: alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, and heterocyclyalkyl-, wherein each of said R⁹ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl- and heterocyclyalkyl- is optionally substituted with 1-3 independently selected R²¹ groups;

R¹⁰ is selected from the group consisting of: a bond, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl-, heterocyclyalkenyl-,

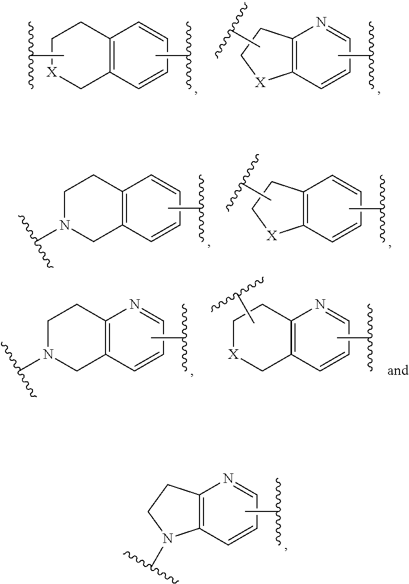

wherein X is selected from the group consisting of: O, —N(R¹⁴)— or —S—; and wherein each of said R¹⁰ moieties is optionally substituted with 1-3 independently selected R²¹ groups;

R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁶)R¹⁶, and —P(O)(OR¹⁶)(OR¹⁶);

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;

Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl; —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

two R¹⁸ moieties on adjacent carbons can be linked together to form a

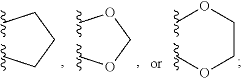

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

Each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁶, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁶)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups; and

Each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

Those skilled in the art will appreciate that when W is —S(O)—, the —S(O)— moiety can be:

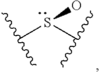

or the —S(O)— moiety can be;

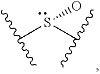

In another embodiment of this invention, R² and R³ taken together, along with the atoms to which they are bound, form a ring selected from the group consisting of:
(a) a 5 to 6 membered heterocycloalkyl ring, said heterocycloalkyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
   wherein said ring is optionally substituted with 1-5 independently selected R²¹ groups.

In another embodiment of this invention, R² and R³ taken together, along with the atoms to which they are bound, form a 5 to 6 membered heterocycloalkyl ring, said heterocycloalkyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and wherein said ring is optionally substituted with 1-5 independently selected R²¹ groups.

In another embodiment of this invention, R² and R³ taken together, along with the atoms to which they are bound, form a 5 to 6 membered heterocycloalkenyl ring, said heterocycloalkenyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and wherein said ring is optionally substituted with 1-5 independently selected R²¹ groups.

In another embodiment of this invention, R² and R³ taken together along with the atoms to which they are bound, and R¹ and R³ are taken together along with the atoms to which they are bound, form the fused ring moiety:

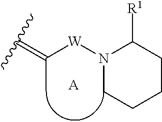

wherein Ring A represents the ring formed when R² and R³ are taken together to form a heterocycloalkyl ring, or a heterocycloalkenyl ring; that is Ring A is a ring selected from the group consisting of:
(a) a 5 to 6 membered heterocycloalkyl ring, said heterocycloalkyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and
   wherein said fused ring moiety is optionally substituted with 1-5 independently selected R²¹ groups.

In another embodiment of this invention, R² and R³ taken together along with the atoms to which they are bound, and R¹ and R³ are taken together along with the atoms to which they are bound, form the fused ring moiety:

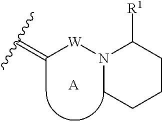

wherein Ring A is a 5 to 6 membered heterocycloalkyl ring, said heterocycloalkyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and wherein said fused ring moiety is optionally substituted with 1-5 independently selected R²¹ groups.

In another embodiment of this invention, R² and R³ taken together along with the atoms to which they are bound, and R¹ and R³ are taken together along with the atoms to which they are bound, form the fused ring moiety:

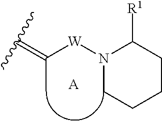

wherein Ring A is a 5 to 6 membered heterocycloalkenyl ring, said heterocycloalkenyl ring optionally comprising, in addition to W and in addition to the N adjacent to W, at least one (e.g., one) other heteroatom independently selected from the group consisting of: —O—, —NR¹⁴—, —S(O)—, —S(O)₂, and —C(O)—, and wherein said fused ring moiety is optionally substituted with 1-5 independently selected R²¹ groups.

In another embodiment of this invention, R¹ and R³ taken together with the atoms to which they are bound form a fused benzoheterocycloalkyl (i.e., benzofusedheterocycloalkyl) ring, wherein said fused ring is optionally substituted with 1-5 independently selected R²¹ groups.

In another embodiment of this invention no fused rings are formed by: (1) R² and R³, and (2) R² and R³, and Wand R³, and (3) R¹ and R³.

In another embodiment R² and R³ can be taken together with the atoms to which they are bound to form a five or six membered heterocycloalkyl (heterocyclyl) ring, or a five or six membered heterocycloalkenyl (heterocyclenyl) ring. Each substitutable carbon atom of the ring is optionally substituted with one or two independently selected R²¹ groups. A substitutable nitrogen atom in the ring is optionally substituted with an R¹⁴ group. Examples of compounds of formula (I) wherein R² and R³ are taken together to form a ring include compounds of formulas (IA) to (IH), (IJ) and (IK).

Thus, another embodiment of the compounds of formula (I) is directed to the compounds of formula (IA):

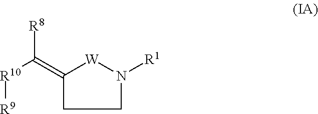

wherein the substituents are as defined for formula (I).

Another embodiment of the compounds of formula (I) is directed to the compounds of formula (IB):

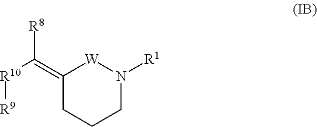

Another embodiment of the compounds of formula (I) is directed to the compounds of formula (IC):

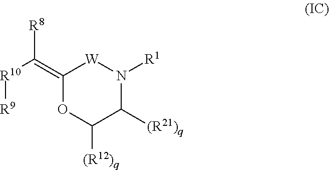

wherein each q is independently 0, 1 or 2, and each R²¹ group is independently selected, and wherein two R²¹ groups on one carbon can be taken together to form a ═O moiety or an ═NR¹⁵ moiety, and wherein all substituents are as defined for formula (I).

Another embodiment of the compounds of formula (I) is directed to the compounds of formula (ID):

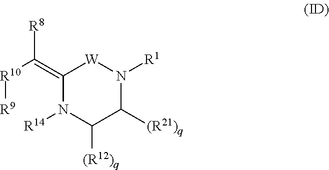

wherein each q is independently 0, 1 or 2, and each R²¹ group is independently selected, and wherein two R²¹ groups on one carbon can be taken together to form a ═O moiety or an ═NR¹⁵ moiety, and wherein all substituents are as defined for formula (I).

Another embodiment of the compounds of formula (I) is directed to the compounds of formula (IE):

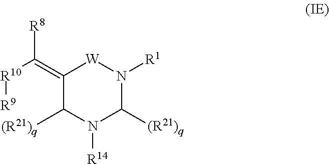

wherein each q is independently 0, 1 or 2, and each R²¹ group is independently selected, and wherein two R²¹ groups on one carbon can be taken together to form a ═O moiety or an ═NR¹⁵ moiety, and wherein all substituents are as defined for formula (I).

Another embodiment of the compounds of formula (I) is directed to the compounds of formula (IF):

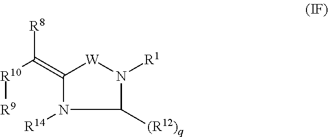

wherein q is 0, 1 or 2, and each R²¹ group is independently selected, and wherein two R²¹ groups on one carbon can be taken together to form a ═O moiety or an ═NR¹⁵ moiety, and wherein all substituents are as defined for formula (I).

Another embodiment of the compounds of formula (I) is directed to the compounds of formula (IG):

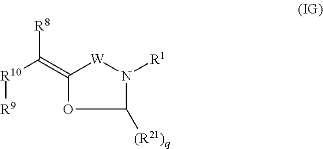

wherein q is 0, 1 or 2, and each R²¹ group is independently selected, and wherein two R²¹ groups on one carbon can be taken together to form a ═O moiety or an ═NR¹⁵ moiety, and wherein all substituents are as defined for formula (I).

Another embodiment of this invention is directed to a compound of formula (I).

Another embodiment of this invention is directed to a pharmaceutically acceptable salt of a compound of formula (I).

Another embodiment of this invention is directed to a pharmaceutically acceptable ester of a compound of formula (I).

Another embodiment of this invention is directed to a solvate of a compound of formula (I).

Another embodiment of this invention is directed to a compound of formula (IA).

Another embodiment of this invention is directed to a pharmaceutically acceptable salt of a compound of formula (IA).

Another embodiment of this invention is directed to a pharmaceutically acceptable ester of a compound of formula (IA).

Another embodiment of this invention is directed to a solvate of a compound of formula (IA).

Another embodiment of this invention is directed to a compound of formula (IB).

Another embodiment of this invention is directed to a pharmaceutically acceptable salt of a compound of formula (IB).

Another embodiment of this invention is directed to a pharmaceutically acceptable ester of a compound of formula (IB).

Another embodiment of this invention is directed to a solvate of a compound of formula (IB).

Another embodiment of this invention is directed to a compound of formula (I) in isolated form.

Another embodiment of this invention is directed to a compound of formula (I) in pure form.

Another embodiment of this invention is directed to a compound of formula (I) in pure and isolated form.

Another embodiment of this invention is directed to a compound of formula (IA) in isolated form.

Another embodiment of this invention is directed to a compound of formula (IA) in pure form.

Another embodiment of this invention is directed to a compound of formula (IA) in pure and isolated form.

Another embodiment of this invention is directed to a compound of formula (IB) in isolated form.

Another embodiment of this invention is directed to a compound of formula (IB) in pure form.

Another embodiment of this invention is directed to a compound of formula (IB) in pure and isolated form.

Another embodiment of this invention is directed to a compound selected from the group consisting of compounds 1.0 to 17.0.

Another embodiment of this invention is directed to a pharmaceutically acceptable salt of a compound selected from the group consisting of compounds 1.0 to 17.0.

Another embodiment of this invention is directed to a pharmaceutically acceptable ester of a compound selected from the group consisting of compounds 1.0 to 17.0.

Another embodiment of this invention is directed to a solvate of a compound selected from the group consisting of compounds 1.0 to 17.0.

Another embodiment is directed to a pharmaceutical composition comprising an effective amount of one or more (e.g., one) compounds of formula (I) and a pharmaceutically acceptable carrier.

Another embodiment is directed to a pharmaceutical composition comprising an effective amount of a pharmaceutically acceptable salt of one or more (e.g., one) compounds of formula (I) and a pharmaceutically acceptable carrier.

Another embodiment is directed to a pharmaceutical composition comprising an effective amount of a pharmaceutically acceptable ester of one or more (e.g., one) compounds of formula (I) and a pharmaceutically acceptable carrier.

Another embodiment is directed to a pharmaceutical composition comprising an effective amount of a solvate of one or more (e.g., one) compounds of formula (I) and a pharmaceutically acceptable carrier.

Another embodiment is directed to a pharmaceutical composition comprising an effective amount of one or more (e.g., one) compounds of formula (IA) and a pharmaceutically acceptable carrier.

Another embodiment is directed to a pharmaceutical composition comprising an effective amount of a pharmaceutically acceptable salt of one or more (e.g., one) compounds of formula (IA) and a pharmaceutically acceptable carrier.

Another embodiment is directed to a pharmaceutical composition comprising an effective amount of a pharmaceutically acceptable ester of one or more (e.g., one) compounds of formula (IA) and a pharmaceutically acceptable carrier.

Another embodiment is directed to a pharmaceutical composition comprising an effective amount of a solvate of one or more (e.g., one) compounds of formula (IA) and a pharmaceutically acceptable carrier.

Another embodiment is directed to a pharmaceutical composition comprising an effective amount of one or more (e.g., one) compounds of formula (IB) and a pharmaceutically acceptable carrier.

Another embodiment is directed to a pharmaceutical composition comprising an effective amount of a pharmaceutically acceptable salt of one or more (e.g., one) compounds of formula (IB) and a pharmaceutically acceptable carrier.

Another embodiment is directed to a pharmaceutical composition comprising an effective amount of a pharmaceutically acceptable ester of one or more (e.g., one) compounds of formula (IB) and a pharmaceutically acceptable carrier.

Another embodiment is directed to a pharmaceutical composition comprising an effective amount of a solvate of one or more (e.g., one) compounds of formula (IB) and a pharmaceutically acceptable carrier.

Another embodiment is directed to a pharmaceutical composition comprising an effective amount of one or more (e.g., one) compounds selected from the group consisting of compounds 1.0 to 17.0 and a pharmaceutically acceptable carrier.

This invention also provides combinations (i.e., pharmaceutical compositions) comprising an effective (i.e., therapeutically effective) amount of one or more (e.g., one) compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

Another embodiment of this invention is directed to a pharmaceutical composition comprising an effective amount of one or more (e.g., one) compounds of formula (I), and an effective amount of one or more (e.g., one) other pharmaceutically active ingredients (e.g. drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds selected from the group consisting of compounds 1.0 to 17.0, and effective amount of one or more (e.g., one) other therapeutically effective pharmaceutical active ingredients (e.g., drugs), and a pharmaceutically acceptable carrier. Examples of the other drugs include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

As used herein, “other pharmaceutically active ingredients” includes, for example, pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a pharmaceutical composition comprising an effective amount of one or more (e.g., one) compounds of formula (I), and effective amount of one or more BACE inhibitors, and a pharmaceutically acceptable carrier.

Another embodiment of this invention is directed to a pharmaceutical composition comprising an effective amount of one or more (e.g., one) compounds of formula (IA), and effective amount of one or more BACE inhibitors, and a pharmaceutically acceptable carrier.

Another embodiment of this invention is directed to a pharmaceutical composition comprising an effective amount of one or more (e.g., one) compounds of formula (IB), and effective amount of one or more BACE inhibitors, and a pharmaceutically acceptable carrier.

Another embodiment of this invention is directed to a pharmaceutical composition comprising an effective amount of one or more (e.g., one) compounds selected from the group consisting of compounds 1.0 to 17.0, and effective amount of one or more BACE inhibitors, and a pharmaceutically acceptable carrier.

Another embodiment of this invention is directed to a pharmaceutical composition comprising an effective amount of one or more (e.g., one) compounds of formula (I), and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of compounds 1.0 to 17.0, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of formula (I), and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds selected from the group consisting of compounds 1.0 to 17.0, and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds of formula (I), and effective amount of Exelon (rivastigmine), and a pharmaceutically acceptable carrier.

Another embodiment of this invention is directed to a pharmaceutical composition comprising an effective amount of one or more (e.g., one) compounds of formula (I), and effective amount of Cognex (tacrine), and a pharmaceutically acceptable carrier.

Another embodiment of this invention is directed to a pharmaceutical composition comprising an effective amount of one or more (e.g., one) compounds of formula (I), and effective amount of a Tau kinase inhibitor, and a pharmaceutically acceptable carrier.

Another embodiment of this invention is directed to a pharmaceutical composition comprising an effective amount of one or more (e.g., one) compounds of formula (I), and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula (I), and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula (I), and effective amount of one or more APP ligands, and a pharmaceutically acceptable carrier.

Another embodiment of this invention is directed to a pharmaceutical composition comprising an effective amount of one or more (e.g., one) compounds of formula (I), and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula (I), and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula (I), and effective amount of one or more fibrates (for example, clofibrate, Clofibride, Etofibrate, Aluminium Clofibrate), and a pharmaceutically acceptable carrier.

Another embodiment of this invention is directed to a pharmaceutical composition comprising an effective amount of one or more (e.g., one) compounds of formula (I), and effective amount of one or more LXR agonists, and a pharmaceutically acceptable carrier.

Another embodiment of this invention is directed to a pharmaceutical composition comprising an effective amount of one or more (e.g., one) compounds of formula (I), and effective amount of one or more LRP mimics, and a pharmaceutically acceptable carrier.

Another embodiment of this invention is directed to a pharmaceutical composition comprising an effective amount of one or more (e.g., one) compounds of formula (I), and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula (I), and effective amount of one or more nicotinic receptor agonists, and a pharmaceutically acceptable carrier.

Another embodiment of this invention is directed to a pharmaceutical composition comprising an effective amount of one or more (e.g., one) compounds of formula (I), and effective amount of one or more H3 receptor antagonists, and a pharmaceutically acceptable carrier.

Another embodiment of this invention is directed to a pharmaceutical composition comprising an effective amount of one or more (e.g., one) compounds of formula (I), and effective amount of one or more histone deacetylase inhibitors, and a pharmaceutically acceptable carrier.

Another embodiment of this invention is directed to a pharmaceutical composition comprising an effective amount of one or more (e.g., one) compounds of formula (I), and effective amount of one or more hsp90 inhibitors, and a pharmaceutically acceptable carrier.

Another embodiment of this invention is directed to a pharmaceutical composition comprising an effective amount of one or more (e.g., one) compounds of formula (I), and effective amount of one or more ml muscarinic receptor agonists, and a pharmaceutically acceptable carrier.

Another embodiment of this invention is directed to a pharmaceutical composition comprising an effective amount of one or more (e.g., one) compounds of formula (I), and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula (I), and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula (I), and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula (I), and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula (I), and effective amount of one or more PAI-1 inhibitors, and a pharmaceutically acceptable carrier.

Another embodiment of this invention is directed to a pharmaceutical composition comprising an effective amount of one or more (e.g., one) compounds of formula (I), and effective amount of one or more agents that can induce Abeta efflux such as gelsolin, and a pharmaceutically acceptable carrier.

Other embodiments of this invention are directed to any one of the above pharmaceutical compositions wherein the compounds of formula (I) are selected from the group consisting of compounds of the formula: (1.0) to (17.0).

Another embodiment of this invention is directed to a pharmaceutical composition comprising an effective amount of at least one (e.g., one) compound of formula (I) selected from the group consisting of compounds of the formula: (1.0) to (17.0), and a pharmaceutically acceptable carrier.

Another embodiment of this invention is directed to a pharmaceutical composition comprising an effective amount of a compound of formula (I) selected from the group consisting of compounds of the formula: (1.0) to (17.0), and a pharmaceutically acceptable carrier.

Another embodiment of this invention is directed to a pharmaceutical composition comprising an effective amount of a pharmaceutically acceptable salt of at least one (e.g., one) compound of formula (I) selected from the group consisting of compounds of the formula: (1.0) to (17.0), and a pharmaceutically acceptable carrier.

Another embodiment of this invention is directed to a pharmaceutical composition comprising an effective amount of a pharmaceutically acceptable salt of a compound of formula (I) selected from the group consisting of compounds of the formula: (1.0) to (17.0), and a pharmaceutically acceptable carrier.

Another embodiment of this invention is directed to a pharmaceutical composition comprising an effective amount of a pharmaceutically acceptable ester of at least one (e.g., one) compound of formula (I) selected from the group consisting of compounds of the formula: (1.0) to (17.0), and a pharmaceutically acceptable carrier.

Another embodiment of this invention is directed to a pharmaceutical composition comprising an effective amount of a pharmaceutically acceptable ester of a compound of formula (I) selected from the group consisting of compounds of the formula: (1.0) to (17.0), and a pharmaceutically acceptable carrier.

Another embodiment of this invention is directed to a pharmaceutical composition comprising an effective amount of a solvate of at least one (e.g., one) compound of formula (I) selected from the group consisting of compounds of the formula: (1.0) to (17.0), and a pharmaceutically acceptable carrier.

Another embodiment of this invention is directed to a pharmaceutical composition comprising an effective amount of a solvate of a compound of formula (I) selected from the group consisting of compounds of the formula: (1.0) to (17.0), and a pharmaceutically acceptable carrier.

The compounds of formula (I) can be useful as gamma secretase modulators and can be useful in the treatment and prevention of diseases such as, for example, central nervous system disorders (such as Alzheimers disease and Downs Syndrome), and treating mild cognitive impairment, glaucoma, cerebral amyloid angiopathy, stroke, dementia, microgliosis, brain inflammation, and olfactory function loss.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment.

Another embodiment of this invention is directed to a method of treating one or more neurodegenerative diseases, comprising administering an effective (i.e., therapeutically effective) amount of one or more (e.g., one) compounds of formula (I) to a patient in need of treatment.

Another embodiment of this invention is directed to a method of treating one or more neurodegenerative diseases, comprising administering an effective (i.e., therapeutically effective) amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment.

Another embodiment of this invention is directed to a method of treating Alzheimer's disease, comprising administering an effective (i.e., therapeutically effective) amount of one or more (e.g., one) compounds of formula (I) to a patient in need of treatment.

Another embodiment of this invention is directed to a method of treating Alzheimer's disease, comprising administering an effective (i.e., therapeutically effective) amount of a compound of formula (I) to a patient in need of treatment.

This invention also provides a method of treating Downs syndrome, comprising administering an effective (i.e., therapeutically effective) amount of one or more (e.g., one) compounds of formula (I) to a patient in need of treatment.

This invention also provides a method of treating Downs syndrome, comprising administering an effective (i.e., therapeutically effective) amount of a compound of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment.

Another embodiment of this invention is directed to a method of treating mild cognitive impairment, comprising administering an effective amount of one or more (e.g., one) compounds of formula (I) to a patient in need of treatment.

Another embodiment of this invention is directed to a method of treating glaucoma, comprising administering an effective amount of one or more (e.g., one) compounds of formula (I) to a patient in need of treatment.

Another embodiment of this invention is directed to a method of treating cerebral amyloid angiopathy, comprising administering an effective amount of one or more (e.g., one) compounds of formula (I) to a patient in need of treatment.

Another embodiment of this invention is directed to a method of treating stroke, comprising administering an effective amount of one or more (e.g., one) compounds of formula (I) to a patient in need of treatment.

Another embodiment of this invention is directed to a method of treating dementia, comprising administering an effective amount of one or more (e.g., one) compounds of formula (I) to a patient in need of treatment.

Another embodiment of this invention is directed to a method of treating microgliosis, comprising administering an effective amount of one or more (e.g., one) compounds of formula (I) to a patient in need of treatment.

Another embodiment of this invention is directed to a method of treating brain inflammation, comprising administering an effective amount of one or more (e.g., one) compounds of formula (I) to a patient in need of treatment.

Another embodiment of this invention is directed to a method of treating olfactory function loss, comprising administering an effective amount of one or more (e.g., one) compounds of formula (I) to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein an effective amount of the compound of formula (I) is used in combination with an effective amount of one or more other pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Other embodiments of this invention are directed to any one of the methods of treatment, or methods of inhibiting, described herein, wherein an effective amount of the compound of formula (I) is used in combination with an effective amount of one or more other pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; and PDE-10 inhibitors.

Other embodiments of this invention are directed to any one of the methods of treatment, or methods of inhibiting, described herein, wherein an effective amount of the compound of formula (I) is used in combination with an effective amount of one or more other pharmaceutically active ingredients selected from the group consisting of: Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1, mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Thus, other embodiments of this invention are directed to any one of the methods of treatment, or methods of inhibiting, described herein, wherein the compound of formula (I) is used in combination with an effective amount of one or more other pharmaceutically active ingredients selected from the group consisting of: BACE inhibitors (beta secretase inhibitors), muscarinic antagonists (e.g., m₁ or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe). In one embodiment one or more compounds of formula (I) are used wherein R² and R³ do not form a ring. In one embodiment one or more compounds of formula (I) are used wherein R² and R³, and R¹ and R³ do not form a ring. In one embodiment one or more compounds of formula (I) are used wherein R¹ and R³ do not form a ring. In one embodiment one or more compounds of formula (I) are used wherein: (1) R² and R³ do not form a ring, and (2) R² and R³, and R¹ and R³ do not form a ring, and (3) R¹ and R³ do not form a ring. In another embodiment one or more compounds of formula (IA) are used. In another embodiment one or more compounds of formula (IB) are used.

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Alzheimer's disease, comprising administering an effective (i.e., therapeutically effective) amount of a compound of formula I, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Alzheimer's disease, comprising administering an effective (i.e., therapeutically effective) amount of one or more (e.g., one) compounds of formula (I), in combination with an effective (i.e., therapeutically effective) amount of one or more compounds selected from the group consisting of Aβ antibody inhibitors, gamma secretase inhibitors and beta secretase inhibitors.

This invention also provides a method of treating Alzheimer's disease, comprising administering an effective (i.e., therapeutically effective) amount of one or more (e.g., one) compounds of formula (I), in combination with an effective (i.e., therapeutically effective) amount of one or more BACE inhibitors.

Another embodiment of this invention is directed to a method of treating Alzheimer's disease, comprising administering an effective amount of one or more compounds of formula (I), in combination with an effective amount of Exelon (rivastigmine).

Another embodiment of this invention is directed to a method of treating Alzheimer's disease, comprising administering an effective amount of one or more compounds of formula (I), in combination with an effective amount of Cognex (tacrine).

Another embodiment of this invention is directed to a method of treating Alzheimer's disease, comprising administering an effective amount of one or more compounds of formula (I), in combination with an effective amount of a Tau kinase inhibitor.

Another embodiment of this invention is directed to a method of treating Alzheimer's disease, comprising administering an effective amount of one or more compounds of formula (I), in combination with an effective amount of one or more Tau kinase inhibitor (e.g., GSK3beta inhibitor, cdk5 inhibitor, ERK inhibitor).

Another embodiment of this invention is directed to a method of treating Alzheimer's disease, comprising administering an effective amount of one or more compounds of formula (I), in combination with an effective amount of one anti-Abeta vaccination (active immunization).

Another embodiment of this invention is directed to a method of treating Alzheimer's disease, comprising administering an effective amount of one or more compounds of formula (I), in combination with an effective amount of one or more APP ligands.

Another embodiment of this invention is directed to a method of treating Alzheimer's disease, comprising administering an effective amount of one or more compounds of formula (I), in combination with an effective amount of one or more agents that upregulate insulin degrading enzyme and/or neprilysin.

Another embodiment of this invention is directed to a method of treating Alzheimer's disease, comprising administering an effective amount of one or more compounds of formula (I), in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

Another embodiment of this invention is directed to a method of treating Alzheimer's disease, comprising administering an effective amount of one or more compounds of formula (I), in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula (I), in combination with an effective amount of one or more LXR agonists.

Another embodiment of this invention is directed to a method of treating Alzheimer's disease, comprising administering an effective amount of one or more compounds of formula (I), in combination with an effective amount of one or more LRP mimics.

Another embodiment of this invention is directed to a method of treating Alzheimer's disease, comprising administering an effective amount of one or more compounds of formula (I), in combination with an effective amount of one or more 5-HT6 receptor antagonists.

Another embodiment of this invention is directed to a method of treating Alzheimer's disease, comprising administering an effective amount of one or more compounds of formula (I), in combination with an effective amount of one or more nicotinic receptor agonists.

Another embodiment of this invention is directed to a method of treating Alzheimer's disease, comprising administering an effective amount of one or more compounds of formula (I), in combination with an effective amount of one or more H3 receptor antagonists.

Another embodiment of this invention is directed to a method of treating Alzheimer's disease, comprising administering an effective amount of one or more compounds of formula (I), in combination with an effective amount of one or more histone deacetylase inhibitors.

Another embodiment of this invention is directed to a method of treating Alzheimer's disease, comprising administering an effective amount of one or more compounds of formula (I), in combination with an effective amount of one or more hsp90 inhibitors.

Another embodiment of this invention is directed to a method of treating Alzheimer's disease, comprising administering an effective amount of one or more compounds of formula (I), in combination with an effective amount of one or more ml muscarinic receptor agonists.

Another embodiment of this invention is directed to a method of treating Alzheimer's disease, comprising administering an effective amount of one or more compounds of formula (I), in combination with an effective amount of one or more 5-HT6 receptor antagonists, mGluR1, mGluR5, or positive allosteric modulators or agonists.

Another embodiment of this invention is directed to a method of treating Alzheimer's disease, comprising administering an effective amount of one or more compounds of formula (I), in combination with an effective amount of one or more mGluR2/3 antagonists.

Another embodiment of this invention is directed to a method of treating Alzheimer's disease, comprising administering an effective amount of one or more compounds of formula (I), in combination with an effective amount of one or more anti-inflammatory agents that can reduce neuroinflammation.

Another embodiment of this invention is directed to a method of treating Alzheimer's disease, comprising administering an effective amount of one or more compounds of formula (I), in combination with an effective amount of one or more Prostaglandin EP2 receptor antagonists.

Another embodiment of this invention is directed to a method of treating Alzheimer's disease, comprising administering an effective amount of one or more compounds of formula (I), in combination with an effective amount of one or more PAI-1 inhibitors.

Another embodiment of this invention is directed to a method of treating Alzheimer's disease, comprising administering an effective amount of one or more compounds of formula (I), in combination with an effective amount of one or more agents that can induce Abeta efflux such as gelsolin.

This invention also provides a method of treating Downs syndrome, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Other embodiments of this invention are directed to any one of the methods described above wherein the compound of formula (I) is a compound selected from the group consisting of compounds 1.0 to 17.0.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

In the embodiments described above wherein a compound of formula (I) is used, examples of the compounds of formula (I) include: (a) in one example, compounds of formula (I) wherein R² and R³ do not form a ring, (b) in another example, compounds wherein the compound of formula (I) is a compound of formula (IA), (c) in another example, compounds wherein the compound of formula (I) is a compound of formula (IB), (d) in another example wherein R² and R³, and R¹ and R³ do not form a ring, (e) in another example wherein R¹ and R³ do not form a ring, and (f) in another example wherein (i) R² and R³ do not form a ring, and (ii) R² and R³, and R¹ and R³ do not form a ring, and (iii) R¹ and R³ do not form a ring

In the embodiments described above wherein a compound of formula (I) is used, examples of the compounds of formula (I) include: (a) in one example, compounds of formula (I) wherein R² and R³ form a ring, (b) in another example wherein R² and R³, and R¹ and R³ form a fused ring moiety, and (c) in another example wherein R¹ and R³ form a ring.

Other embodiments of this invention are directed to any one of the embodiments above that are directed to formulas (IA) or (IB) wherein a compound of formula (IA.1), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) is used instead of (IA) or (IB).

Other embodiments of this invention are directed to any one of the embodiments above that are directed to formula (I) wherein a compound of formula (IA.1), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), is used instead of (I), (IL) or (IM)

Other embodiments of this invention are directed to any one of the embodiments above that are directed to formulas (IA) or (IB), wherein a compound of formula (IA.1), (IB.1), (IC), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein the bond between the carbon to which R⁸ is bound and the ring carbon is a single bond, that is the moiety

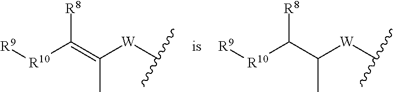

is used instead of (IA) or (IB).

Other embodiments of this invention are directed to any one of the embodiments above that are directed to formula (I), wherein a compound of formula (IA.1), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein the bond between the carbon to which R⁸ is bound and the ring carbon is a single bond, that is the moiety

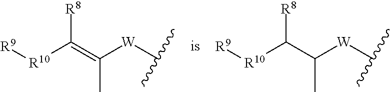

is used instead of (I).

Compounds of formula (I) include compounds of formula (IA.1):

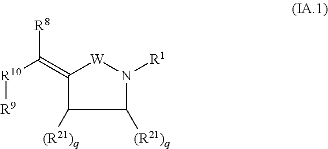

wherein each q is independently 0, 1 or 2, and each R²¹ group is independently selected, and wherein two R²¹ groups on one carbon can be taken together to form a ═O moiety or an ═NR¹⁵ moiety, and wherein all substituents are as defined for formula (I).

Compounds of formula (I) include compounds of formula (IB.1):

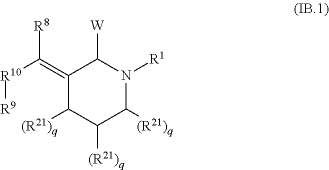

wherein each q is independently 0, 1 or 2, and each R²¹ group is independently selected, and wherein two R²¹ groups on one carbon can be taken together to form a ═O moiety or an ═NR¹⁵ moiety, and wherein all substituents are as defined for formula (I).

Compounds of formula (IC) include compounds of formula (IC.1):

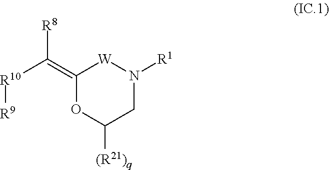

wherein q is 0, 1 or 2, and each R²¹ group is independently selected, and wherein all substituents are as defined for formula (I).

Compounds of formula (IC) include compounds of formula (IC.2):

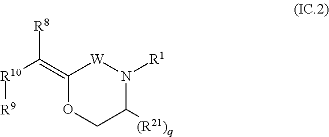

wherein q is 0, 1 or 2, and each R²¹ group is independently selected, and wherein all substituents are as defined for formula (I).

Compounds of formula (ID) include compounds of formula (ID.1):

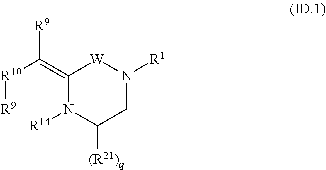

wherein q is 0, 1 or 2, and each R²¹ group is independently selected, and wherein all substituents are as defined for formula (I).

Compounds of formula (ID) include compounds of formula (ID.2):

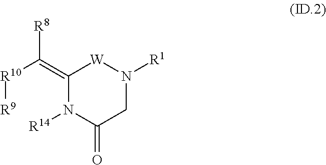

wherein all substituents are as defined for formula (I).

Compounds of formula (ID) include compounds of formula (ID.3):

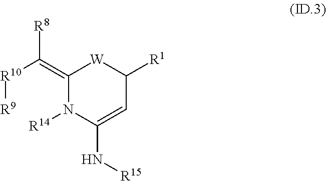

wherein all substituents are as defined for formula (I).

Those skilled in the art will appreciate that compounds of formula (ID.3) include the tautomer of formula (ID.4):

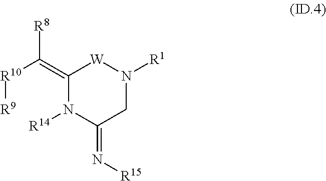

wherein all substituents are as defined for formula (I).

Compounds of formula (ID) include compounds of formula (ID.5):

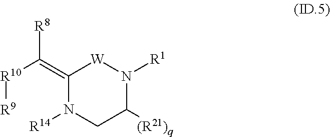

wherein q is 0, 1 or 2, and each R²¹ group is independently selected, and wherein all substituents are as defined for formula (I).

Compounds of formula (ID) include compounds of formula (ID.6):

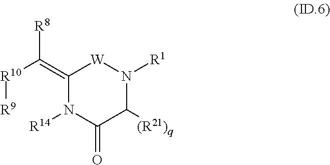

wherein q is 0, 1 or 2, and each R²¹ group is independently selected, and wherein all substituents are as defined for formula (I).

Compounds of formula (ID) include compounds of formula (ID.7):

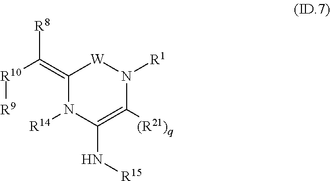

wherein q is 0, 1 or 2, and each R²¹ group is independently selected, and wherein all substituents are as defined for formula (I).

Compounds of formula (ID) include compounds of formula (ID.8):

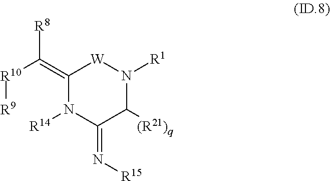

wherein q is 0, 1 or 2, and each R²¹ group is independently selected, and wherein all substituents are as defined for formula (I).

Compounds of formula (IE) include compounds of formula (IE.1):

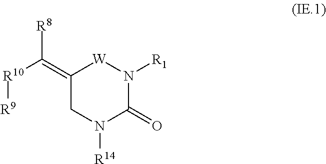

wherein all substituents are as defined for formula (I).

Compounds of formula (IE) include compounds of formula (IE.2):

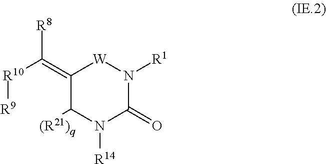

wherein q is 0, 1 or 2, and each R²¹ group is independently selected, and wherein all substituents are as defined for formula (I).

Compounds of formula (IF) include compounds of formula (IF.1):

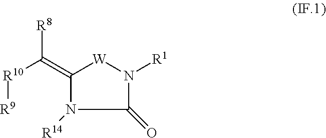

wherein all substituents are as defined for formula (I).

Compounds of formula (IF) include compounds of formula (IF.2):

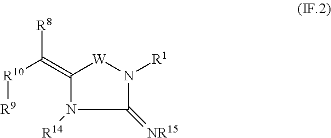

wherein all substituents are as defined for formula (I).

Compounds of formula (IG) include compounds of formula (IG.1):

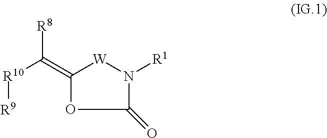

wherein all substituents are as defined for formula (I).

Compounds of formula (IG) include compounds of formula (IG.2):

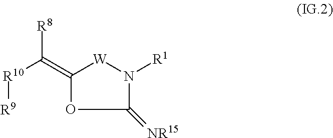

wherein all substituents are as defined for formula (I).

Compounds of formula (I) include compounds of formula (IH):

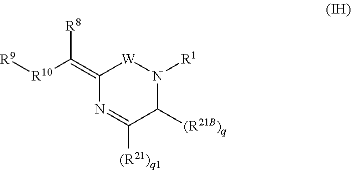

wherein q is 0, 1 or 2, and q1 is 0 or 1, and wherein each R^{21B} group is independently selected, and wherein R^{21B} is defined the same as R²¹, and wherein two R^{21B} groups can be taken together to form a ═O moiety or an ═NR¹⁵ moiety, and wherein all substituents are as defined for formula (I).

Compounds of formula (I) include compounds of formula (IJ):

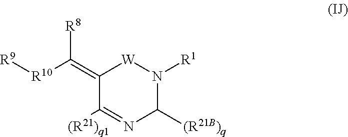

wherein q is 0, 1 or 2, and q1 is 0 or 1, and wherein each R^{21B} group is independently selected, and wherein R^{21B} is defined the same as R²¹, and wherein two R^{21B} groups can be taken together to form a ═O moiety or an ═NR¹⁵ moiety, and wherein all substituents are as defined for formula (I).

Compounds of formula (I) include compounds of formula (IK):

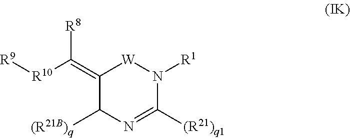

wherein q is 0, 1 or 2, and q1 is 0 or 1, and wherein each R^{21B} group is independently selected, and wherein R^{21B} is defined the same as R²¹, and wherein two R^{21B} groups can be taken together to form a ═O moiety or an ═NR¹⁵ moiety, and wherein all substituents are as defined for formula (I).

Compounds of formula (I) include compounds of formula (IL):

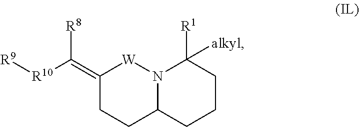

wherein the fused ring moiety:

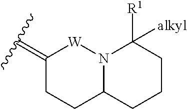

is optionally substituted with 1 to 5 independently selected R²¹ groups, and wherein in one example, said fused ring moiety is not substituted with R²¹ groups.

Compounds of formula (I) include compounds of formula (IL):

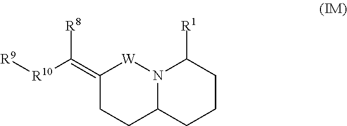

wherein the fused ring moiety:

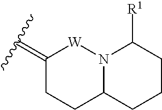

is optionally substituted with 1 to 5 independently selected R²¹ groups, and wherein in one example, said fused ring moiety is not substituted with R²¹ groups.

Compounds of formula (A) include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein the bond between the carbon to which R⁸ is bound and the ring carbon is a single bond. That is compounds of formula (A) include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein the moiety

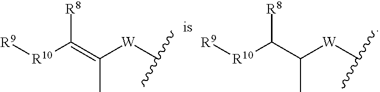

Compounds of this invention include compounds selected from the group consisting of: (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) and (IM) wherein W is —S(O)₂—.

Compounds of this invention include compounds selected from the group consisting of: (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) and (IM) wherein W is —S(O)—. Compounds of this invention include compounds of formula (IA) wherein W is —S(O)₂—.

Compounds of this invention include compounds of formula (IA) wherein W is —S(O)₂—.

Compounds of this invention include compounds of formula (IA.1) wherein W is —S(O)₂—.

Compounds of this invention include compounds of formula (IB) wherein W is —S(O)₂—.

Compounds of this invention include compounds of formula (IB.1) wherein W is —S(O)₂—.

Compounds of this invention include compounds of formula (IC) wherein W is —S(O)₂—.

Compounds of this invention include compounds of formula (IC.1) wherein W is —S(O)₂—.

Compounds of this invention include compounds of formula (IC.2) wherein W is —S(O)₂—.

Compounds of this invention include compounds of formula (ID) wherein W is —S(O)₂—.

Compounds of this invention include compounds of formula (ID.1) wherein W is —S(O)₂—.

Compounds of this invention include compounds of formula (ID.2) wherein W is —S(O)₂—.

Compounds of this invention include compounds of formula (ID.3) wherein W is —S(O)₂—.

Compounds of this invention include compounds of formula (ID.4) wherein W is —S(O)₂—.

Compounds of this invention include compounds of formula (ID.5) wherein W is —S(O)₂—.

Compounds of this invention include compounds of formula (ID.6) wherein W is —S(O)₂—.

Compounds of this invention include compounds of formula (ID.7) wherein W is —S(O)₂—.

Compounds of this invention include compounds of formula (IE) wherein W is —S(O)₂—.

Compounds of this invention include compounds of formula (IE.1) wherein W is —S(O)₂—.

Compounds of this invention include compounds of formula (IE.2) wherein W is —S(O)₂—.

Compounds of this invention include compounds of formula (IF) wherein W is —S(O)₂—.

Compounds of this invention include compounds of formula (IF.1) wherein W is —S(O)₂—.

Compounds of this invention include compounds of formula (IF.2) wherein W is —S(O)₂—.

Compounds of this invention include compounds of formula (IG) wherein W is —S(O)₂—.

Compounds of this invention include compounds of formula (IG.1) wherein W is —S(O)₂—.

Compounds of this invention include compounds of formula (IG.2) wherein W is —S(O)₂—.

Compounds of this invention include compounds of formula (IH) wherein W is —S(O)₂—.

Compounds of this invention include compounds of formula (IJ) wherein W is —S(O)₂—.

Compounds of this invention include compounds of formula (IK) wherein W is —S(O)₂—.

Compounds of this invention include compounds of formula (IL) wherein W is —S(O)₂—.

Compounds of this invention include compounds of formula (IM) wherein W is —S(O)₂—.

Compounds of this invention include compounds of formula (I) wherein R¹ is selected from the group consisting of: benzofusedcycloalkyl (i.e., fused benzocycloalkyl), fused benzoheterocycloalkyl, fused heteroarylcycloalkyl, fused heteroarylheterocycloalkyl, and wherein said R¹ groups are optionally substituted with 1-5 independently selected R²¹ groups. In one example, the R²¹ groups are halo (e.g., F).

Thus, compounds of this invention include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is selected from the group consisting of: benzofusedcycloalkyl (i.e., fused benzocycloalkyl), fused benzoheterocycloalkyl, fused heteroarylcycloalkyl, fused heteroarylheterocycloalkyl, and wherein said R¹ groups are optionally substituted with 1-5 independently selected R²¹ groups. In one example, the R²¹ groups are halo (e.g., F).

Compounds of this invention also include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is selected from the group consisting of: benzofusedcycloalkyl (i.e., fused benzocycloalkyl), fused benzoheterocycloalkyl, fused heteroarylcycloalkyl, fused heteroarylheterocycloalkyl, wherein said R¹ groups are optionally substituted with 1-5 independently selected R²¹ groups, and wherein W is —S(O)₂—. In one example, the R²¹ groups are halo (e.g., F).

Compounds of this invention also include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.4 (IE.2), (IF), (IF.1), (IF.2), (IG), (IG.1), (IG.2), (IH), (IJ), (IK), (IL) or (IM) wherein R¹ is selected from the group consisting of: benzofusedcycloalkyl (i.e., fused benzocycloalkyl), fused benzoheterocycloalkyl, fused heteroarylcycloalkyl, fused heteroarylheterocycloalkyl, wherein said R¹ groups are optionally substituted with 1-5 independently selected R²¹ groups, and wherein W is —S(O)—. In one example, the R²¹ groups are halo (e.g., F).

Examples of the fused ring R¹ groups include, but are not limited to:

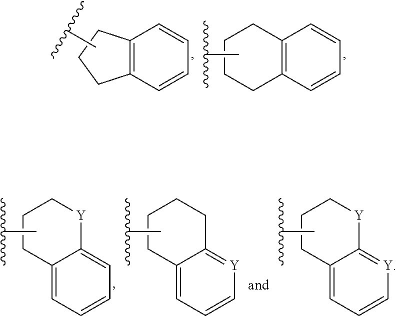

wherein each Y is independently selected from the group consisting of: —O—, —NR¹⁴— and —C(R²¹)_{q}—, wherein q is as defined above (i.e., 0, 1 or 2 and each R²¹ is independently selected), and wherein R¹⁴ and R²¹ are as defined for formula (I). Examples of these R¹ groups include, for example:

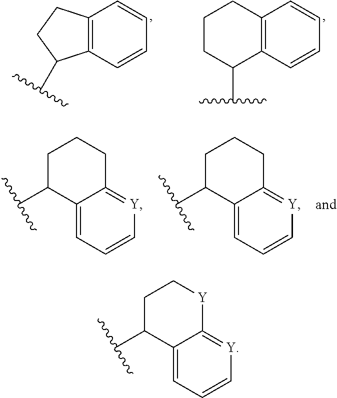

Compounds of formula (I) also include compounds wherein R¹ is an alkyl group (e.g., ethyl) substituted with one R²¹ group. Examples of said R¹ groups include alkyl (e.g., methyl or ethyl) substituted with the R²¹ moiety aryl (e.g., phenyl or naphthyl). Examples of said R¹ groups also include alkyl (e.g., methyl or ethyl) substituted with the R²¹ moiety aryl (e.g., phenyl or naphthyl), which in turn is substituted with one or more (e.g., one or two) independently selected R²² groups (e.g., R²² is halo, such as, for example, F).

Thus, compounds of this invention also include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is an alkyl group (e.g., methyl or ethyl) substituted with the R²¹ group aryl (such as, for example, phenyl or napthyl), or R¹ is an alkyl group (e.g., methyl or ethyl) substituted with the R²¹ moiety aryl (e.g., phenyl or naphthyl) which in turn is substituted with one or more (e.g., one or two) independently selected R²² groups (e.g., R²² is halo, such as, for example, F).

Compounds of this invention also include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is an alkyl group (e.g., methyl or ethyl) substituted with the R²¹ group aryl (such as, for example, phenyl or napthyl), or R¹ is an alkyl group (e.g., methyl or ethyl) substituted with the R²¹ moiety aryl (e.g., phenyl or naphthyl) which in turn is substituted with one or more (e.g., one or two) independently selected R²² groups (e.g., R²² is halo, such as, for example, F), and W is —S(O)₂—

Compounds of this invention also include any one of the compounds of (IA),

(IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is an alkyl group (e.g., methyl or ethyl) substituted with the R²¹ group aryl (such as, for example, phenyl or napthyl), or R¹ is an alkyl group (e.g., methyl or ethyl) substituted with the R²¹ moiety aryl (e.g., phenyl or naphthyl) which in turn is substituted with one or more (e.g., one or two) independently selected R²² groups (e.g., R²² is halo, such as, for example, F), and W is —S(O)—

Examples of the substituted R¹ alkyl groups include, but are not limited to:

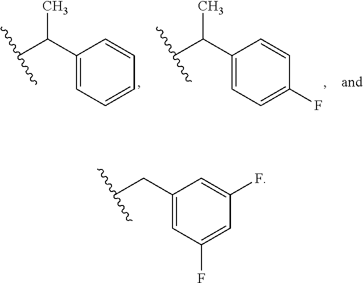

Compounds of this invention include compounds of formula (I) wherein R¹ is a cycloalkyl group (e.g., cyclopropyl or cyclobutyl) substituted with one R²¹ group (e.g., aryl, such as, for example, phenyl), or a cycloalkyl group (e.g., cyclopentyl or cyclohexyl) substituted with one R²¹ group (e.g., aryl, such as, for example, phenyl) which in turn is substituted with one or more (e.g., one or two) independently selected R²² groups (e.g., halo, such as, for example, F). In one example the R²¹ group is bound to the same carbon of the R¹ group that binds the R¹ group to the rest of the molecule.

Thus compounds of this also invention include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is a cycloalkyl group (e.g., e.g., cyclopropyl or cyclobutyl) substituted with the R²¹ group aryl (such as, for example, phenyl), or a cycloalkyl group (e.g., cyclopentyl or cyclohexyl) substituted with the R²¹ group aryl (such as, for example, phenyl) which in turn is substituted with one or more (e.g., one or two) independently selected R²² groups (e.g., halo, such as, for example, F). In one example the R²¹ group is bound to the same carbon of the R¹ group that binds the R¹ group to the rest of the molecule.

Thus compounds of this also invention include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is a cycloalkyl group (e.g., e.g., cyclopropyl or cyclobutyl) substituted with the R²¹ group aryl (such as, for example, phenyl), or a cycloalkyl group (e.g., cyclopentyl or cyclohexyl) substituted with one R²¹ group (e.g., aryl, such as, for example, phenyl) which in turn is substituted with one or more (e.g., one or two) independently selected R²² groups (e.g., halo, such as, for example, F), and W is —S(O)₂—. In one example the R²¹ group is bound to the same carbon of the R¹ group that binds the R¹ group to the rest of the molecule.

Thus compounds of this also invention include any one of the compounds of formulas (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) or (IM) wherein R¹ is a cycloalkyl group (e.g., e.g., cyclopropyl or cyclobutyl) substituted with the R²¹ group aryl (such as, for example, phenyl), or a cycloalkyl group (e.g., cyclopentyl or cyclohexyl) substituted with the R²¹ group aryl (such as, for example, phenyl) which in turn is substituted with one or more (e.g., one or two) independently selected R²² groups (e.g., halo, such as, for example, F), and W is —S(O)—. In one example the R²¹ group is bound to the same carbon of the R¹ group that binds the R¹ group to the rest of the molecule.

Examples of the cycloalkyl R¹ groups include, but are not limited to:

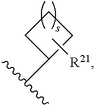

such as, for example,

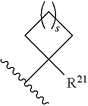

wherein s is 0 (i.e., the ring is cyclopropyl), or 1 (i.e., the ring is cyclobutyl). Examples of these R¹ groups include, but are not limited to:

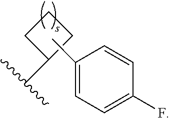

such as, for example,

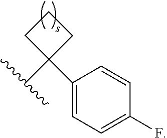

wherein s is 0 (i.e., the ring is cyclopropyl), or 1 (i.e., the ring is cyclobutyl).

Compounds of this invention include compounds of formula (I) wherein R¹ is

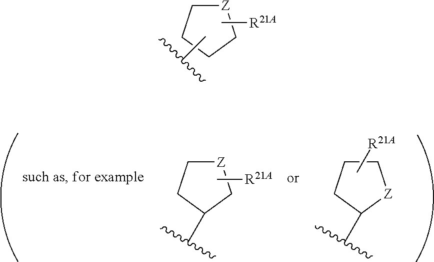

wherein Z is selected from the group consisting of: (1) —O—, (2) —NR¹⁴—, (3) —C(R²¹)_{q}— wherein q is 0, 1 or 2, and each R²¹ is independently selected, (4) —C(R²¹)_{q}—C(R²¹)_{q}— wherein each q is independently 0, 1 or 2 and each R²¹ is independently selected, (5) —(C(R²¹)_{q})_{q}—(C(R²¹)_{q})_{q}— wherein each q is independently 0, 1 or 2, and each R²¹ is independently selected, and (6) —(C(R²¹)_{q})_{q}—N(R¹⁴)—(C(R²¹)_{q})_{q}— wherein each q is independently 0, 1 or 2, and each R²¹ is independently selected. R^{21A} is defined the same as R²¹ for formula (I). Examples of R^{21A} include, but are not limited to, aryl (e.g., phenyl) and aryl (e.g., phenyl) substituted with one or more (e.g., one or two, or one) independently selected R²² groups (e.g., halo, such as, for example, F). Examples of this R¹ include, but are not limited to:

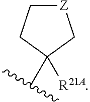

Thus, examples of this R¹ group include, but are not limited to:

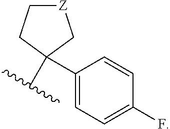

Examples of R¹ also include, but are not limited to:

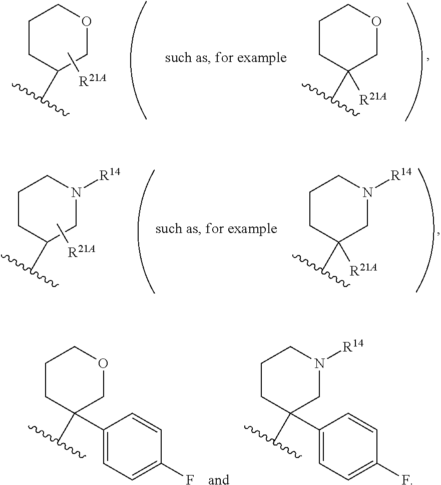

Examples of the R¹ group

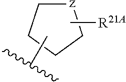

also include, but are not limited to:

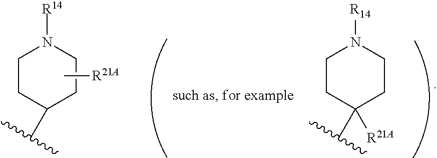

Examples of the R¹ group

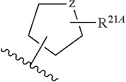

also include, but are not limited to:

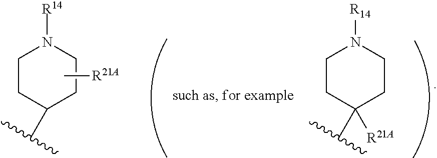

Examples of the R¹ group

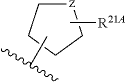

also include, but are not limited to:

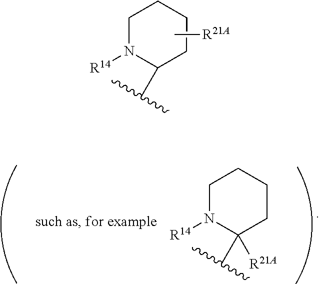

Examples of the R¹ group

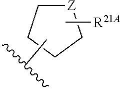

also include, but are not limited to:

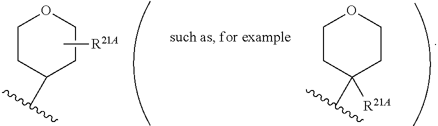

Examples of the R¹ group

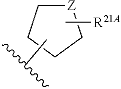

also include, but are not limited to:

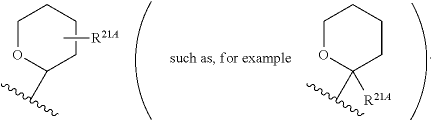

Thus, compounds of this invention include compounds wherein R¹ is as described in this paragraph and W is —S(O)₂—. Compounds of this invention include compounds wherein R¹ is as described in this paragraph and W is —S(O)—.

Compounds of this invention also include compounds of formula (I) wherein R⁸ is H, R¹⁰ is aryl (e.g., phenyl) or aryl (e.g., phenyl) substituted with one or more (e.g., one or two, or one) R²¹ groups (e.g., —OR¹⁵, wherein, for example, R¹⁵ is alkyl, such as, for example, methyl), and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Thus, compounds of this invention include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein R⁸ is H, R¹⁰ is aryl (e.g., phenyl) or aryl (e.g., phenyl) substituted with one or more (e.g., one or two, or one) R²¹ groups (e.g., —OR¹⁵, wherein, for example, R¹⁵ is alkyl, such as, for example, methyl), and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Compounds of this invention also include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein R⁸ is H, R¹⁰ is aryl (e.g., phenyl) or aryl (e.g., phenyl) substituted with one or more (e.g., one or two, or one) R²¹ groups (e.g., —OR¹⁵, wherein, for example, R¹⁵ is alkyl, such as, for example, methyl), R⁹ is heteroaryl (e.g., imidazolyl) or heteroaryl (e.g., imidazolyl) substituted with one or more (e.g., one or two, or one) R²¹ groups (e.g., alkyl, such as, for example, methyl), and W is —S(O)₂—

Compounds of this invention also include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein R⁸ is H, R¹⁰ is aryl (e.g., phenyl) or aryl (e.g., phenyl) substituted with one or more (e.g., one or two, or one) R²¹ groups (e.g., −OR¹⁵, wherein, for example, R¹⁵ is alkyl, such as, for example, methyl), R⁹ is heteroaryl (e.g., imidazolyl) or heteroaryl (e.g., imidazolyl) substituted with one or more (e.g., one or two, or one) R²¹ groups (e.g., alkyl, such as, for example, methyl), and W is —S(O)—

Thus, examples of the

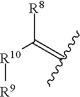

moiety of the compounds of this invention include, but are not limited to:

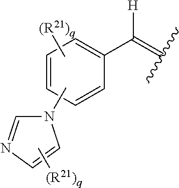

wherein q is 0, 1 or 2, such as, for example,

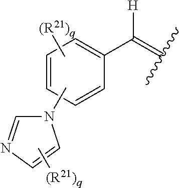

such as, for example,

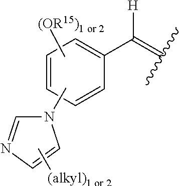

wherein R¹⁵ is alkyl (e.g., methyl), such as, for example,

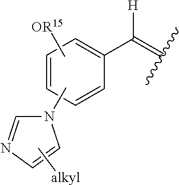

wherein R¹⁵ is alkyl (e.g., methyl), such as, for example,

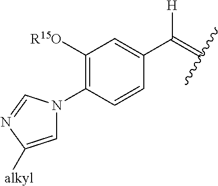

wherein R¹⁵ is alkyl (e.g., methyl), such as, for example,

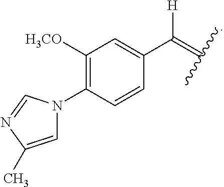

Compounds of this invention also include compounds of formula (I) wherein R⁸ is H, R¹⁰ is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Thus, compounds of this invention include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein R⁸ is H, R¹⁰ is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Compounds of this invention also include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein R⁸ is H, R¹⁰ is heteroaryl or heteroaryl substituted with one or more R²¹ groups, R⁹ is heteroaryl (e.g., imidazolyl) or heteroaryl (e.g., imidazolyl) substituted with one or more (e.g., one or two, or one) R²¹ groups (e.g., alkyl, such as, for example, methyl), and W is —S(O)₂—.

Compounds of this invention also include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein R⁸ is H, R¹⁰ is heteroaryl or heteroaryl substituted with one or more R²¹ groups, R⁹ is heteroaryl (e.g., imidazolyl) or heteroaryl (e.g., imidazolyl) substituted with one or more (e.g., one or two, or one) R²¹ groups (e.g., alkyl, such as, for example, methyl), and W is —S(O)—.

Compounds of this invention also include compounds of formula (I) wherein the moiety:

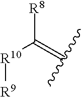

has any one of the definitions described above, and R¹ has any one of the definitions described above, and W is —S(O)₂—, or W is —S(O)—.

Compounds of this invention also include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein the moiety:

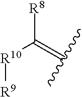

has any one of the definitions described above, and R¹ has any one of the definitions described above, and W is —S(O)₂—.

Compounds of this invention also include any one of the compounds of formulas (IA), (IA.1), (IB), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) wherein the moiety:

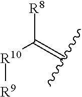

has any one of the definitions described above, and R¹ has any one of the definitions described above, and W is —S(O)—.

In one embodiment of the compounds of formula (I) R² and R³ are not taken together to form a ring.

Thus, in one embodiment of this invention R² is H.

In another embodiment of this invention R³ is H.

In another embodiment of this invention R³ is an alkyl group.

In another embodiment of this invention R³ is methyl:

In another embodiment of this invention R² is H and R³ is H.

In another embodiment of this invention R² is H and R³ is alkyl.

In another embodiment of this invention R² is H and R³ is methyl.

In another embodiment of this invention W is —S(O)—.

In another embodiment of this invention W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention W is —S(O)₂—.

In another embodiment of this invention W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁸ is H.

In another embodiment of this invention R⁸ is H, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment R¹⁰ is aryl substituted with 1 to 3 independently selected R²¹ moieties.

In another embodiment R¹⁰ is aryl substituted with 1 to 3 R²¹ moieties, wherein each R²¹ moiety is the same or different —OR¹⁵ group.

In another embodiment R¹⁰ is aryl substituted with 1 R²¹ moiety.

In another embodiment R¹⁰ is aryl substituted with one R²¹ moiety, and said R²¹ moiety is —OR¹⁵.

In another embodiment R¹⁰ is aryl substituted with one R²¹ moiety, said R²¹ moiety is —OR¹⁵, and said R¹⁵ is alkyl.

In another embodiment R¹⁰ is aryl substituted with one R²¹ moiety, said R²¹ moiety is —OR¹⁵, said R¹⁵ is alkyl, and said alkyl is methyl (i.e., said R²¹ moiety is —OCH₃).

In another embodiment R¹⁰ is phenyl substituted with 1 to 3 independently selected R²¹ moieties.

In another embodiment R¹⁰ is phenyl substituted with 1 to 3 R²¹ moieties, wherein each R²¹ moiety is the same or different —OR¹⁵ group.

In another embodiment R¹⁰ is phenyl substituted with 1 R²¹ moiety.

In another embodiment R¹⁰ is phenyl substituted with one R²¹ moiety, and said R²¹ moiety is —OR¹⁵.

In another embodiment R¹⁰ is phenyl substituted with one R²¹ moiety, said R²¹ moiety is —OR¹⁵, and said R¹⁵ is alkyl.

In another embodiment R¹⁰ is phenyl substituted with one R²¹ moiety, said R²¹ moiety is —OR¹⁵, said R¹⁵ is alkyl, and said alkyl is methyl (i.e., said R²¹ moiety is —OCH₃).

In another embodiment R¹⁰ is:

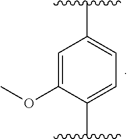

In another embodiment R¹⁰ is:

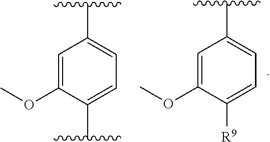

wherein the —R¹⁰—R⁹ moiety is:

In another embodiment R¹⁰ is aryl substituted with 1 to 3 R²¹ moieties, wherein each R²¹ moiety is the same or different halo.

In another embodiment R¹⁰ is aryl substituted with 1 to 3 R²¹ moieties, wherein each R²¹ moiety is F.

In another embodiment R¹⁰ is aryl substituted with one R²¹ moiety, and said R²¹ moiety is halo.

In another embodiment R¹⁰ is aryl substituted with one R²¹ moiety, said R²¹ moiety is -halo, and said halo is F.

In another embodiment R¹⁰ is phenyl substituted with 1 to 3 R²¹ moieties, wherein each R²¹ moiety is the same or different halo.

In another embodiment R¹⁰ is phenyl substituted with 1 to 3 R²¹ moieties, wherein each R²¹ moiety is F.

In another embodiment R¹⁰ is phenyl substituted with one R²¹ moiety, and said R²¹ moiety is halo.

In another embodiment R¹⁰ is phenyl substituted with one R²¹ moiety, said R²¹ moiety is -halo, and said halo is F.

In another embodiment R¹⁰ is:

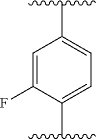

In another embodiment R¹⁰ is:

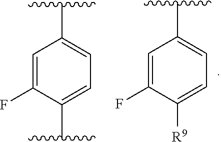

wherein the —R¹⁰—R⁹ moiety is:

In another embodiment, R¹⁰ is unsubstituted heteroaryl.

In another embodiment R¹⁰ is unsubstituted heteroaryl wherein said heteroaryl is pyridyl.

In another embodiment R¹⁰ is:

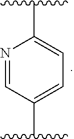

In another embodiment R¹⁰ is:

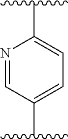

wherein the —R¹⁰—R⁹ moiety is:

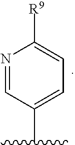

In another embodiment R¹⁰ is selected from the group consisting of:

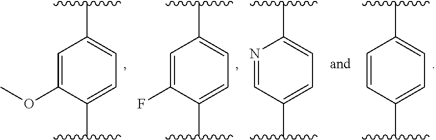

In another embodiment of this invention R¹⁰ is aryl.

In another embodiment of this invention R¹⁰ is aryl, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is aryl, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is phenyl.

In another embodiment of this invention R¹⁰ is phenyl, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is aryl substituted with one or more independently selected (e.g., one) R²¹ groups.

In another embodiment of this invention R¹⁰ is phenyl substituted with one or more (e.g., one) independently selected R²¹ groups.

In another embodiment of this invention R¹⁰ is aryl substituted with one or more (e.g., one) independently selected R²¹ groups, and R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is phenyl substituted with one or more (e.g., one) independently selected R²¹ groups, and R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is phenyl substituted with one or more (e.g., one) independently selected R²¹ groups and R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is aryl substituted with one R²¹ group.

In another embodiment of this invention R¹⁰ is aryl substituted with one R²¹ group, and R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is aryl substituted with one R²¹ group, and R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is phenyl substituted with one R²¹ group.

In another embodiment of this invention R¹⁰ is phenyl substituted with one R²¹ group, and R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is phenyl substituted with one R²¹ group, and R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is aryl substituted with one R²¹ group, wherein said R²¹ group is —OR¹⁵. In one example, R¹⁵ is alkyl. In another example R¹⁵ is methyl.

In another embodiment of this invention R¹⁰ is aryl substituted with one R²¹ group, wherein said R²¹ group is —OR¹⁵. In one example, R¹⁵ is alkyl. In another example R¹⁵ is methyl. In any one of these embodiments R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is aryl substituted with one R²¹ group, wherein said R²¹ group is —OR¹⁵. In one example, R¹⁵ is alkyl. In another example R¹⁵ is methyl. In any one of these embodiments R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is phenyl substituted with one R²¹ group, wherein said R²¹ group is —OR¹⁵. In one example, R¹⁵ is alkyl. In another example R¹⁵ is methyl.

In another embodiment of this invention R¹⁰ is phenyl substituted with one R²¹ group, wherein said R²¹ group is —OR¹⁵. In one example, R¹⁵ is alkyl. In another example R¹⁵ is methyl. In any one of these embodiments R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R¹⁰ is phenyl substituted with one R²¹ group, wherein said R²¹ group is —OR¹⁵. In one example, R¹⁵ is alkyl. In another example R¹⁵ is methyl. In any one of these embodiments R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl.

In another embodiment, R⁹ is unsubstituted heteroaryl.

In another embodiment of this invention R⁹ is selected from the group consisting of heteroaryl and heteroaryl substituted with 1-3 R²¹ groups, and wherein each R²¹ is independently selected.

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxyl, alkoxy, alkyl substituted with halo (e.g., alkyl substituted with F, such as, for example, —CH₂F), and alkyl substituted with —OR¹⁵ (such as, for example, alkyl substituted with —OR¹⁵ wherein R¹⁵ is H, that is, —CH₂OH).

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups.

In another embodiment of this invention R⁹ is selected from the group consisting of imidazolyl and imidazolyl substituted with 1-3 R²¹ groups, and wherein each R²¹ is independently selected.

In another embodiment of this invention R⁹ is imidazolyl substituted with 1-3 R²¹ groups, and wherein each R²¹ is independently selected.

In another embodiment, R⁹ is imidazolyl which is substituted with 1-3 substituents which can be the same or different, each substituent being independently selected from the group consisting of halo, alkyl, CN, NH₂, NH(alkyl), N(alkyl)₂, hydroxyl, alkoxy, alkyl substituted with halo (e.g., alkyl substituted with F, such as, for example, —CH₂F), and alkyl substituted with —OR¹⁵ (such as, for example, alkyl substituted with —OR¹⁵ wherein R¹⁵ is H, that is, —CH₂OH).

In another embodiment, R⁹ is imidazolyl substituted with 1-3 substituents independently selected from the group consisting of halo, alkyl, CN, NH₂, NH(alkyl), N(alkyl)₂, hydroxy and alkoxy groups.

In another embodiment of this invention R⁹ is heteroaryl, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one or more (e.g., one) independently selected R²¹ groups.

In another embodiment of this invention R⁹ is heteroaryl substituted with one or more (e.g., one) independently selected R²¹ groups, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one or more (e.g., one) independently selected R²¹ groups, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one or more (e.g., one) independently selected R²¹ groups, wherein each R²¹ group is the same or different alkyl group (e.g., methyl).

In another embodiment of this invention R⁹ is heteroaryl substituted with one or more (e.g., one) independently selected R²¹ groups, wherein each R²¹ group is the same or different alkyl group (e.g., methyl), R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one or more (e.g., one) independently selected R²¹ groups, wherein each R²¹ group is the same or different alkyl group (e.g., methyl), R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one R²¹ group.

In another embodiment of this invention R⁹ is heteroaryl substituted with one R²¹ group, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one R²¹ group, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one R²¹ group, wherein R²¹ is an alkyl group (e.g., methyl).

In another embodiment of this invention R⁹ is heteroaryl substituted with one R²¹ group, wherein R²¹ is an alkyl group (e.g., methyl), R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl substituted with one R²¹ group, wherein R²¹ is an alkyl group (e.g., methyl), R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment, R⁹ is 4-methyl-imidazol-1-yl.

In another embodiment, R⁹ is 5-chloro-4-methyl-imidazol-1-yl.

In another embodiment, R⁹ is:

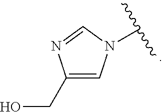

In another embodiment, R⁹ is:

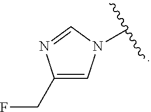

In another embodiment of this invention R⁹ is imidazolyl.

In another embodiment of this invention R⁹ is imidazolyl, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one or more (e.g., one) independently selected R²¹ groups.

In another embodiment of this invention R⁹ is imidazolyl substituted with one or more (e.g., one) independently selected R²¹ groups, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one or more (e.g., one) independently selected R²¹ groups, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one or more (e.g., one) independently selected R²¹ groups, wherein each R²¹ group is the same or different alkyl group (e.g., methyl).

In another embodiment of this invention R⁹ is imidazolyl substituted with one or more (e.g., one) independently selected R²¹ groups, wherein each R²¹ group is the same or different alkyl group (e.g., methyl), R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one or more (e.g., one) independently selected R²¹ groups, wherein each R²¹ group is the same or different alkyl group (e.g., methyl), R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one R²¹ group.

In another embodiment of this invention R⁹ is imidazolyl substituted with one R²¹ group, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one R²¹ group, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one R²¹ group, wherein R²¹ is an alkyl group (e.g., methyl).

In another embodiment of this invention R⁹ is imidazolyl substituted with R²¹ group, wherein R²¹ is an alkyl group (e.g., methyl), R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is imidazolyl substituted with one R²¹ group, wherein R²¹ is an alkyl group (e.g., methyl), R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl).

In another embodiment of this invention R⁹ is heteroaryl, optionally substituted with one or more R²¹ groups, and R¹⁰ is aryl optionally substituted with one or more (e.g., one) R²¹ groups.

In another embodiment of this invention R⁹ is heteroaryl, optionally substituted with one R²¹ group, and R¹⁰ is aryl optionally substituted with one R²¹ group.

In another embodiment of this invention R⁹ is heteroaryl, optionally substituted with one or more R²¹ groups, and R¹⁰ is phenyl optionally substituted with one or more (e.g., one) R²¹ groups.

In another embodiment of this invention R⁹ is heteroaryl, optionally substituted with one R²¹ group, and R¹⁰ is phenyl optionally substituted with one R²¹ group.

In another embodiment of this invention R⁹ is imidazolyl, optionally substituted with one or more R²¹ groups, and R¹⁰ is aryl optionally substituted with one or more (e.g., one) R²¹ groups.

In another embodiment of this invention R⁹ is imidazolyl, optionally substituted with one R²¹ group, and R¹⁰ is aryl optionally substituted with one R²¹ group.

In another embodiment of this invention R⁹ is imidazolyl, optionally substituted with one or more R²¹ groups, and R¹⁰ is phenyl optionally substituted with one or more (e.g., one) R²¹ groups.

In another embodiment of this invention R⁹ is imidazolyl, optionally substituted with one R²¹ group, and R¹⁰ is phenyl optionally substituted with one R²¹ group.

In another embodiment of this invention R⁹ is heteroaryl, optionally substituted with one or more R²¹ groups, R¹⁰ is aryl optionally substituted with one or more (e.g., one) R²¹ groups, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is heteroaryl, optionally substituted with one R²¹ group, and R¹⁰ is aryl optionally substituted with one R²¹ group, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is heteroaryl, optionally substituted with one or more R²¹ groups, and R¹⁰ is phenyl optionally substituted with one or more (e.g., one) R²¹ groups, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is heteroaryl, optionally substituted with one R²¹ group, and R¹⁰ is phenyl optionally substituted with one R²¹ group, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is independently selected from alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one or more R²¹ groups, and R¹⁰ is aryl optionally substituted with one or more (e.g., one) R²¹ groups, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one R²¹ group, and R¹⁰ is aryl optionally substituted with one R²¹ group, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one or more R²¹ groups, and R¹⁰ is phenyl optionally substituted with one or more (e.g., one) R²¹ groups, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one R²¹ group, and R¹⁰ is phenyl optionally substituted with one R²¹ group, R⁸ is H, W is —S(O)—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is independently selected from alkyl. In another example of this embodiment the R²¹ group for R¹⁰ is independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is heteroaryl, optionally substituted with one or more R²¹ groups, R¹⁰ is aryl optionally substituted with one or more (e.g., one) R²¹ groups, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is heteroaryl, optionally substituted with one R²¹ group, and R¹⁰ is aryl optionally substituted with one R²¹ group, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is heteroaryl, optionally substituted with one or more R²¹ groups, and R¹⁰ is phenyl optionally substituted with one or more (e.g., one) R²¹ groups, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is heteroaryl, optionally substituted with one R²¹ group, and R¹⁰ is phenyl optionally substituted with one R²¹ group, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is independently selected from alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one or more R²¹ groups, and R¹⁰ is aryl optionally substituted with one or more (e.g., one) R²¹ groups, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one R²¹ group, and R¹⁰ is aryl optionally substituted with one R²¹ group, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one or more R²¹ groups, and R¹⁰ is phenyl optionally substituted with one or more (e.g., one) R²¹ groups, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is imidazolyl, optionally substituted with one R²¹ group, and R¹⁰ is phenyl optionally substituted with one R²¹ group, R⁸ is H, W is —S(O)₂—, R² and R³ are not taken together to form a ring, and R² and R³ are as described in any one of the embodiments above. For example R² is H and R³ is H, or in another example, R² is H and R³ is alkyl (e.g., methyl). In one example the R²¹ group for R⁹ is independently selected from alkyl. In another example of this embodiment the R²¹ group for R¹⁰ is independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

Other embodiments are directed to any one of the above embodiments wherein R⁹ is:

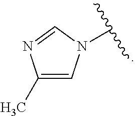

Other embodiments are directed to any one of the above embodiments wherein R¹⁰ is:

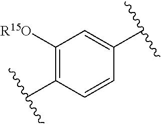

(wherein the —OR¹⁵ is ortho to the carbon to which R⁹ is bound to, i.e., the R⁹—R¹⁰— moiety is:

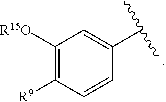

Other embodiments are directed to any one of the above embodiments wherein R¹⁰ is:

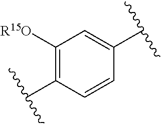

(wherein the —OCH₃ is ortho to the carbon to which R⁹ is bound to, i.e., the R⁹—R¹⁰— moiety is:

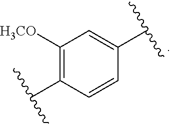

In another embodiment R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

In another embodiment R¹⁰ is selected from the group consisting of phenyl and phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is selected from the group consisting of heteroaryl and heteroaryl substituted with 1-3 R²¹ groups, and the R⁹ group is selected from the group consisting of heteroaryl and heteroaryl substituted with 1-3 R²¹ groups, and wherein each R²¹ is independently selected.

In another embodiment R¹⁰ is selected from the group consisting of pyridyl and pyridyl substituted with 1-3 R²¹ groups, and the R⁹ group is selected from the group consisting of imidazolyl and imidazolyl substituted with 1-3 R²¹ groups, and wherein each R²¹ is independently selected.

In another embodiment R¹⁰ is pyridyl, and the R⁹ group is imidazolyl substituted with 1-3 R²¹ groups, and wherein each R²¹ is independently selected.

In another embodiment the R⁹—R¹⁰— moiety is:

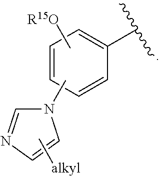

In another embodiment the R⁹—R¹⁰— moiety is:

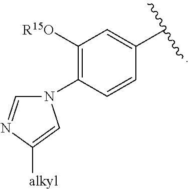

In another embodiment the R⁹—R¹⁰— moiety is:

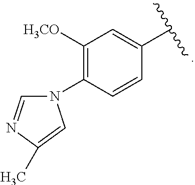

In another embodiment the R⁹—R¹⁰— moiety is:

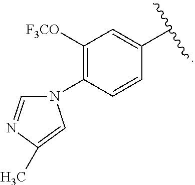

In another embodiment the R⁹—R¹⁰— moiety is:

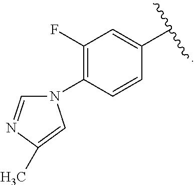

In another embodiment R⁹—R¹⁰— moiety is:

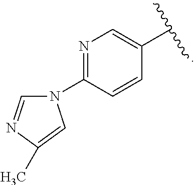

In another embodiment R⁹—R¹⁰— moiety is:

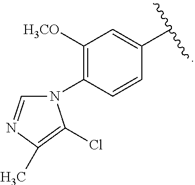

In another embodiment of this invention R¹ is benzofusedcycloalkyl.

In another embodiment of this invention R¹ is:

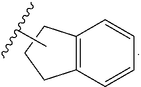

In another embodiment of this invention R¹ is:

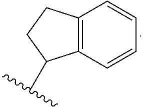

In another embodiment of this invention R¹ is:

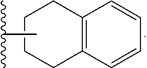

In another embodiment of this invention R¹ is:

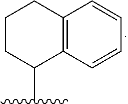

Other embodiments of this invention are directed to any one of the embodiments described above for R², R³, R⁸, W, R⁹, and R¹⁰ (either individually or in combination) wherein R¹ is benzofusedcycloalkyl.

Other embodiments of this invention are directed to any one of the embodiments described above for R², R³, R⁸, W, R⁹, and R¹⁰ (either individually or in combination) wherein R¹ is:

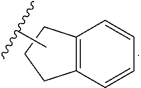

Other embodiments of this invention are directed to any one of the embodiments described above for R², R³, R⁸, W, R⁹, and R¹⁰ (either individually or in combination) wherein R¹ is:

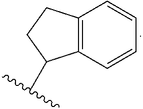

Other embodiments of this invention are directed to any one of the embodiments described above for R², R³, R⁸, W, R⁹, and R¹⁰ (either individually or in combination) wherein R¹ is:

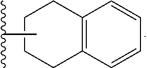

Other embodiments of this invention are directed to any one of the embodiments described above for R², R³, R⁸, W, R⁹, and R¹⁰ (either individually or in combination) wherein R¹ is:

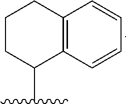

In another embodiment of this invention R¹ is alkyl substituted with one R²¹ group.

In another embodiment of this invention R¹ is alkyl substituted with one R²¹ group, and said alkyl is

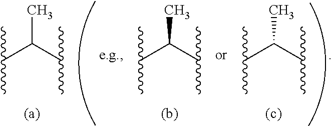

In another embodiment of this invention R¹ is alkyl (e.g., (a), (b) or (c) described above) substituted with one R²¹ group wherein said R²¹ group is aryl.

In another embodiment of this invention R¹ is alkyl (e.g., (a), (b) or (c) described above) substituted with one R²¹ group wherein said R²¹ group is phenyl.

In another embodiment of this invention R¹ is alkyl (e.g., (a), (b) or (c) described above) substituted with one R²¹ group wherein said R²¹ group is naphthyl.

In another embodiment of this invention R¹ is alkyl substituted with one R²¹ group, and said R²¹ group is substituted with two independently selected R²² groups.

In another embodiment of this invention R¹ is alkyl substituted with one R²¹ group, and said R²¹ group is substituted with one R²² group.

In another embodiment of this invention R¹ is alkyl substituted with one R²¹ group, wherein said alkyl group is (a) (e.g., (b) or (c)), as described above, and said R²¹ group is substituted with two independently selected R²² groups.

In another embodiment of this invention R¹ is alkyl substituted with one R²¹ group, wherein said alkyl group is (a) (e.g., (b) or (c)), as described above, and said R²¹ group is substituted with one R²² group.

In another embodiment of this invention R¹ is alkyl substituted with one R²¹ group, wherein said R²¹ group is aryl, and said R²¹ group is substituted with two independently selected R²² groups.

In another embodiment of this invention R¹ is alkyl substituted with one R²¹ group, wherein said R²¹ group is aryl, and said R²¹ group is substituted with one R²² group.

In another embodiment of this invention R¹ is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of this invention R¹ is alkyl substituted with one R²¹ group, wherein said R²¹ group is aryl, wherein said alkyl group is (a) (e.g., (b) or (c)), as described above, and said R²¹ group is substituted with one R²² group.

In another embodiment of this invention R¹ is alkyl substituted with one R²¹ group, wherein said R²¹ group is aryl, said R²¹ group is substituted with two independently selected R²² groups, and each R²² is halo.

In another embodiment of this invention R¹ is alkyl substituted with one R²¹ group, wherein said R²¹ group is aryl, and said R²¹ group is substituted with one R²² group, and said R²² is halo.

In another embodiment of this invention R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of this invention R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is halo.

In another embodiment of this invention R¹ is alkyl substituted with one R²¹ group, wherein said R²¹ group is aryl, said R²¹ group is substituted with two independently selected R²² groups, and each R²² is F.

In another embodiment of this invention R¹ is alkyl substituted with one R²¹ group, wherein said R²¹ group is aryl, and said R²¹ group is substituted with one R²² group, and said R²² is F.

In another embodiment of this invention R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of this invention R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of this invention R¹ is:

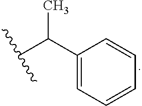

In another embodiment of this invention R¹ is:

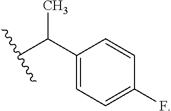

In another embodiment of this invention R¹ is:

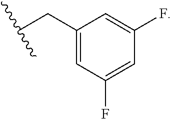

Other embodiments of this invention are directed to any one of the above embodiments described above for R², R³, R⁸, W, R⁹, and R¹⁰ (either individually or in combination) wherein R¹ is alkyl substituted with one R²¹ group, and said alkyl is

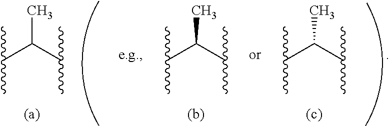

Other embodiments of this invention are directed to any one of the above embodiments described above for R², R³, R⁸, W, R⁹, and R¹⁰ (either individually or in combination) wherein R¹ is alkyl (e.g., (a), (b) or (c) described above) substituted with one R²¹ group wherein said R²¹ group is aryl.

Other embodiments of this invention are directed to any one of the above embodiments described above for R², R³, R⁸, W, R⁹, and R¹⁰ (either individually or in combination) wherein R¹ is alkyl (e.g., (a), (b) or (c) described above) substituted with one R²¹ group wherein said R²¹ group is phenyl.

Other embodiments of this invention are directed to any one of the above embodiments described above for R², R³, R⁸, W, R⁹, and R¹⁰ (either individually or in combination) wherein R¹ is alkyl (e.g., (a), (b) or (c) described above) substituted with one R²¹ group wherein said R²¹ group is naphthyl.

Other embodiments of this invention are directed to any one of the above embodiments described above for R², R³, R⁸, W, R⁹, and R¹⁰ (either individually or in combination) wherein R¹ is alkyl substituted with one R²¹ group, and said R²¹ group is substituted with two independently selected R²² groups.

Other embodiments of this invention are directed to any one of the above embodiments described above for R², R³, R⁸, W, R⁹, and R¹⁰ (either individually or in combination) wherein R¹ is alkyl substituted with one R²¹ group, and said R²¹ group is substituted with one R²² group.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said alkyl group is (a) (e.g., (b) or (c)), as described above, and said R²¹ group is substituted with two independently selected R²² groups.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said alkyl group is (a) (e.g., (b) or (c)), as described above, and said R²¹ group is substituted with one R²² group.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, and said R²¹ group is substituted with two independently selected R²² groups.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, and said R²¹ group is substituted with one R²² group.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, said alkyl group is (a) (e.g., (b) or (c)), as described above, and said R²¹ group is substituted with two independently selected R²² groups.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, wherein said alkyl group is (a) (e.g., (b) or (c)), as described above, and said R²¹ group is substituted with one R²² group.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, said R²¹ group is substituted with two independently selected R²² groups, and each R²² is halo.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, and said R²¹ group is substituted with one R²² group, and said R²² is halo.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is halo.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, said R²¹ group is substituted with two independently selected R²² groups, and each R²² is F.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, and said R²¹ group is substituted with one R²² group, and said R²² is F.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

Other embodiments of this invention are directed to any one of the above embodiments described above for R², R³, R⁸, W, R⁹, and R¹⁰ (either individually or in combination) wherein R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

Other embodiments of this invention are directed to any one of the above embodiments described above for R², R³, R⁸, W, R⁹, and R¹⁰ (either individually or in combination) wherein R¹ is:

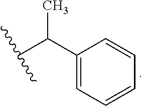

Other embodiments of this invention are directed to any one of the above embodiments described above for R², R³, R⁸, W, R⁹, and R¹⁰ (either individually or in combination) wherein R¹ is:

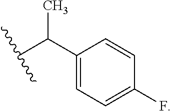

Other embodiments of this invention are directed to any one of the above embodiments described above for R², R³, R⁸, W, R⁹, and R¹⁰ (either individually or in combination) wherein R¹ is:

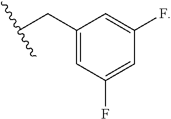

Another embodiment of this invention is directed to compounds of formula (I) wherein R² and R³ taken together with the with the atoms to which they are bound, form a 5 to 6 membered ring.

Thus, another embodiment of this invention is directed to a compound of formula (I) having the formula (IA):

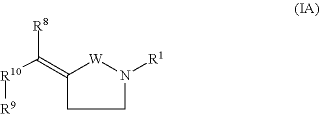

wherein R¹, R⁸, R⁹, R¹⁰, and W are as defined in formula (I).

Another embodiment of this invention is directed to a compound of formula (I) having the formula (IB):

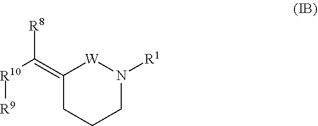

wherein R¹, R⁸, R⁹, R¹⁰, and W are as defined in formula (I).

In another embodiment of the compounds of formula (IA) W is —S(O)—.

In another embodiment of the compounds of formula (IA) W is —S(O)₂—.

In another embodiment of the compounds of formula (IA) R⁸ is H.

In another embodiment of the compounds of formula (IA) R⁸ is H, and W is —S(O)—.

In another embodiment of the compounds of formula (IA) R⁸ is H, and W is —S(O)₂—.

In another embodiment of the compounds of formula (IA) R¹⁰ is aryl.

In another embodiment of the compounds of formula (IA) R¹⁰ is aryl, R⁸ is H, and W is —S(O)—.

In another embodiment of the compounds of formula (IA) R¹⁰ is aryl, R⁸ is H, and W is —S(O)₂—.

In another embodiment of the compounds of formula (IA) R¹⁰ is phenyl.

In another embodiment of the compounds of formula (IA) R¹⁰ is phenyl, R⁸ is H, and W is —S(O)—.

In another embodiment of the compounds of formula (IA) R¹⁰ is aryl substituted with one or more independently selected (e.g., one) R²¹ groups.

In another embodiment of the compounds of formula (IA) R¹⁰ is phenyl substituted with one or more (e.g., one) independently selected R²¹ groups.

In another embodiment of the compounds of formula (IA) R¹⁰ is aryl substituted with one or more (e.g., one) independently selected R²¹ groups, and R⁸ is H, and W is —S(O)—.

In another embodiment of the compounds of formula (IA) R¹⁰ is phenyl substituted with one or more (e.g., one) independently selected R²¹ groups, and R⁸ is H, and W is —S(O)—.

In another embodiment of the compounds of formula (IA) R¹⁰ is phenyl substituted with one or more (e.g., one) independently selected R²¹ groups and R⁸ is H, and W is —S(O)₂—.

In another embodiment of the compounds of formula (IA) R¹⁰ is aryl substituted with one R²¹ group.

In another embodiment of the compounds of formula (IA) R¹⁰ is aryl substituted with one R²¹ group, R⁸ is H, and W is —S(O)—.

In another embodiment of the compounds of formula (IA) R¹⁰ is aryl substituted with one R²¹ group, R⁸ is H, and W is —S(O)₂—.

In another embodiment of the compounds of formula (IA) R¹⁰ is phenyl substituted with one R²¹ group.

In another embodiment of the compounds of formula (IA) R¹⁰ is phenyl substituted with one R²¹ group, R⁵ is H, and W is —S(O)—.

In another embodiment of the compounds of formula (IA) R¹⁰ is phenyl substituted with one R²¹ group, R⁸ is H, and W is —S(O)₂—.

In another embodiment of the compounds of formula (IA) R¹⁰ is aryl substituted with one R²¹ group, wherein said R²¹ group is —OR¹⁵. In one example, R¹⁵ is alkyl. In another example R¹⁵ is methyl.

In another embodiment of the compounds of formula (IA) R¹⁰ is aryl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)—.

In another embodiment of the compounds of formula (IA) R¹⁰ is aryl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)₂—.

In another embodiment of the compounds of formula (IA) R¹⁰ is phenyl substituted with one R²¹ group, wherein said R²¹ group is —OR¹⁵. In one example, R¹⁵ is alkyl. In another example R¹⁵ is methyl.

In another embodiment of the compounds of formula (IA) R¹⁰ is phenyl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)—.

In another embodiment of the compounds of formula (IA) R¹⁰ is phenyl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)₂—.

In another embodiment of the compounds of formula (IA) R⁹ is heteroaryl.

In another embodiment of the compounds of formula (IA) R⁹ is heteroaryl, R⁸ is H, and W is —S(O)—.

In another embodiment of the compounds of formula (IA) R⁹ is heteroaryl, R⁸ is H, and W is —S(O)₂—.

In another embodiment of the compounds of formula (IA) R⁹ is heteroaryl substituted with one or more (e.g., one) independently selected R²¹ groups.

In another embodiment of the compounds of formula (IA) R⁹ is heteroaryl substituted with one or more (e.g., one) independently selected R²¹ groups, R⁸ is H, and W is —S(O)—.

In another embodiment of the compounds of formula (IA) R⁹ is heteroaryl substituted with one or more (e.g., one) independently selected R²¹ groups, R⁸ is H, and W is —S(O)₂—.

In another embodiment of the compounds of formula (IA) R⁹ is heteroaryl substituted with one or more (e.g., one) independently selected R²¹ groups, wherein each R²¹ group is the same or different alkyl group (e.g., methyl).

In another embodiment of the compounds of formula (IA) R⁹ is heteroaryl substituted with one or more (e.g., one) independently selected R²¹ groups, wherein each R²¹ group is the same or different alkyl group (e.g., methyl), R⁸ is H, and W is —S(O)—.

In another embodiment of the compounds of formula (IA) R⁹ is heteroaryl substituted with one or more (e.g., one) independently selected R²¹ groups, wherein each R²¹ group is the same or different alkyl group (e.g., methyl), R⁸ is H, and W is —S(O)₂—.

In another embodiment of the compounds of formula (IA) R⁹ is heteroaryl substituted with one R²¹ group.

In another embodiment of the compounds of formula (IA) R⁹ is heteroaryl substituted with one R²¹ group, R⁸ is H, and W is —S(O)—.

In another embodiment of the compounds of formula (IA) R⁹ is heteroaryl substituted with one R²¹ group, R⁸ is H, and W is —S(O)₂—.

In another embodiment of the compounds of formula (IA) R⁹ is heteroaryl substituted with one R²¹ group, wherein R²¹ is an alkyl group (e.g., methyl).

In another embodiment of the compounds of formula (IA) R⁹ is heteroaryl substituted with R²¹ group, wherein R²¹ is an alkyl group (e.g., methyl), R⁸ is H, and W is —S(O)—.

In another embodiment of the compounds of formula (IA) R⁹ is heteroaryl substituted with one R²¹ group, wherein R²¹ is an alkyl group (e.g., methyl), R⁸ is H, and W is —S(O)₂—.

In another embodiment of the compounds of formula (IA) R⁹ is imidazolyl.

In another embodiment of the compounds of formula (IA) R⁹ is imidazolyl, R⁸ is H, and W is —S(O)—.

In another embodiment of the compounds of formula (IA) R⁹ is imidazolyl, R⁸ is H, and W is —S(O)₂—.

In another embodiment of the compounds of formula (IA) R⁹ is imidazolyl substituted with one or more (e.g., one) independently selected R²¹ groups.

In another embodiment of the compounds of formula (IA) R⁹ is imidazolyl substituted with one or more (e.g., one) independently selected R²¹ groups, R⁸ is H, and W is —S(O)—.

In another embodiment of the compounds of formula (IA) R⁹ is imidazolyl substituted with one or more (e.g., one) independently selected R²¹ groups, R⁸ is H, and W is —S(O)₂—.

In another embodiment of the compounds of formula (IA) R⁹ is imidazolyl substituted with one or more (e.g., one) independently selected R²¹ groups, wherein each R²¹ group is the same or different alkyl group (e.g., methyl).

In another embodiment of the compounds of formula (IA) R⁹ is imidazolyl substituted with one or more (e.g., one) independently selected R²¹ groups, wherein each R²¹ group is the same or different alkyl group (e.g., methyl), R⁸ is H, and W is —S(O)—.

In another embodiment of the compounds of formula (IA) R⁹ is imidazolyl substituted with one or more (e.g., one) independently selected R²¹ groups, wherein each R²¹ group is the same or different alkyl group (e.g., methyl), R⁸ is H, and W is —S(O)₂—.

In another embodiment of the compounds of formula (IA) R⁹ is imidazolyl substituted with one R²¹ group.

In another embodiment of the compounds of formula (IA) R⁹ is imidazolyl substituted with one R²¹ group, R⁸ is H, and W is —S(O)—.

In another embodiment of the compounds of formula (IA) R⁹ is imidazolyl substituted with one R²¹ group, R⁸ is H, and W is —S(O)₂—.

In another embodiment of the compounds of formula (IA) R⁹ is imidazolyl substituted with one R²¹ group, wherein R²¹ is an alkyl group (e.g., methyl).

In another embodiment of the compounds of formula (IA) R⁹ is imidazolyl substituted with R²¹ group, wherein R²¹ is an alkyl group (e.g., methyl), R⁸ is H, and W is —S(O)—.

In another embodiment of the compounds of formula (IA) R⁹ is imidazolyl substituted with one R²¹ group, wherein R²¹ is an alkyl group (e.g., methyl), R⁸ is H, and W is —S(O)₂—.

In another embodiment of the compounds of formula (IA) R⁹ is heteroaryl, optionally substituted with one or more R²¹ groups, and R¹⁰ is aryl optionally substituted with one or more (e.g., one) R²¹ groups.

In another embodiment of the compounds of formula (IA) R⁹ is heteroaryl, optionally substituted with one R²¹ group, and R¹⁰ is aryl optionally substituted with one R²¹ group.

In another embodiment of the compounds of formula (IA) R⁹ is heteroaryl, optionally substituted with one or more R²¹ groups, and R¹⁰ is phenyl optionally substituted with one or more (e.g., one) R²¹ groups.

In another embodiment of the compounds of formula (IA) R⁹ is heteroaryl, optionally substituted with one R²¹ group, and R¹⁰ is phenyl optionally substituted with one R²¹ group.

In another embodiment of the compounds of formula (IA) R⁹ is imidazolyl, optionally substituted with one or more R²¹ groups, and R¹⁰ is aryl optionally substituted with one or more (e.g., one) R²¹ groups.

In another embodiment of the compounds of formula (IA) R⁹ is imidazolyl, optionally substituted with one R²¹ group, and R¹⁰ is aryl optionally substituted with one R²¹ group.

In another embodiment of the compounds of formula (IA) R⁹ is imidazolyl, optionally substituted with one or more R²¹ groups, and R¹⁰ is phenyl optionally substituted with one or more (e.g., one) R²¹ groups.

In another embodiment of the compounds of formula (IA) R⁹ is imidazolyl, optionally substituted with one R²¹ group, and R¹⁰ is phenyl optionally substituted with one R²¹ group.

In another embodiment of the compounds of formula (IA) R⁹ is heteroaryl, optionally substituted with one or more R²¹ groups, R¹⁰ is aryl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is heteroaryl, optionally substituted with one R²¹ group, and R¹⁰ is aryl optionally substituted with one R²¹ group, R⁸ is H, and W is —S(O)—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is heteroaryl, optionally substituted with one or more R²¹ groups, and R¹⁰ is phenyl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is heteroaryl, optionally substituted with one R²¹ group, and R¹⁰ is phenyl optionally substituted with one R²¹ group, R⁸ is H, and W is —S(O)—. In one example the R²¹ group for R⁹ is independently selected from alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one or more R²¹ groups, and R¹⁰ is aryl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one R²¹ group, and R¹⁰ is aryl optionally substituted with one R²¹ group, R⁸ is H, and W is —S(O)—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one or more R²¹ groups, and R¹⁰ is phenyl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one R²¹ group, and R¹⁰ is phenyl optionally substituted with one R²¹ group, R⁸ is H, and W is —S(O)—. In one example the R²¹ group for R⁹ is independently selected from alkyl. In another example of this embodiment the R²¹ group for R¹⁰ is independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is heteroaryl, optionally substituted with one or more R²¹ groups, R¹⁰ is aryl optionally substituted with one or more (e.g., one) R²¹ groups, R⁸ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is heteroaryl, optionally substituted with one R²¹ group, and R¹⁰ is aryl optionally substituted with one R²¹ group, R⁸ is H, and W is —S(O)₂—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is heteroaryl, optionally substituted with one or more R²¹ groups, and R¹⁰ is phenyl optionally substituted with one or more (e.g., one) R²¹ groups, R⁸ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is heteroaryl, optionally substituted with one R²¹ group, and R¹⁰ is phenyl optionally substituted with one R²¹ group, R⁸ is H, and W is —S(O)₂—. In one example the R²¹ group for R⁹ is independently selected from alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one or more R²¹ groups, and R¹⁰ is aryl optionally substituted with one or more (e.g., one) R²¹ groups, R⁵ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one R²¹ group, and R¹⁰ is aryl optionally substituted with one R²¹ group, R⁸ is H, and W is —S(O)₂—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one or more R²¹ groups, and R¹⁰ is phenyl optionally substituted with one or more (e.g., one) R²¹ groups, R⁸ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IA) R⁹ is imidazolyl, optionally substituted with one R²¹ group, and R¹⁰ is phenyl optionally substituted with one R²¹ group, R⁸ is H, and W is —S(O)₂—. In one example the R²¹ group for R⁹ is independently selected from alkyl. In another example of this embodiment the R²¹ group for R¹⁰ is independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

Other embodiments of the compounds of formula (IA) are directed to any one of the above embodiments for formula (IA) wherein R⁹ is:

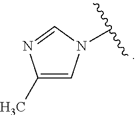

Other embodiments of the compounds of formula (IA) are directed to any one of the above embodiments for the compounds of formula (IA) wherein R¹⁰ is:

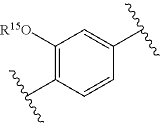

(wherein the —OR¹⁵ is ortho to the carbon to which R⁹ is bound to, i.e., the R⁹—R¹⁰— moiety is:

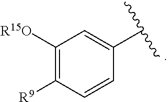

Other embodiments for the compounds of formula (IA) are directed to any one of the above embodiments for formula (IA) wherein R¹⁰ is:

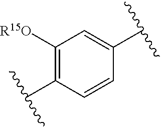

(wherein the —OCH₃ is ortho to the carbon to which R⁹ is bound to, i.e., the R⁹—R¹⁰— moiety is:

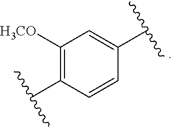

In another embodiment of the compounds of formula (IA) R¹ is benzofusedcycloalkyl.

In another embodiment of the compounds of formula (IA) R¹ is:

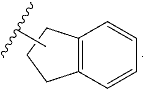

In another embodiment of the compounds of formula (IA) R¹ is:

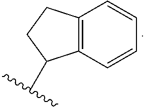

In another embodiment of the compounds of formula (IA) R¹ is:

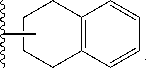

In another embodiment of the compounds of formula (IA) R¹ is:

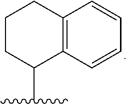

Other embodiments of the compounds of formula (IA) are directed to any one of the embodiments described above for formula (IA) wherein R¹ is benzofusedcycloalkyl.

Other embodiments of the compounds of formula (IA) are directed to any one of the embodiments described above for formula (IA) wherein R¹ is:

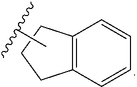

Other embodiments of formula (IA) are directed to any one of the embodiments described above for formula (IA) wherein R¹ is:

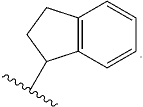

Other embodiments of formula (IA) are directed to any one of the embodiments described above for formula (IA) wherein R¹ is:

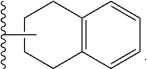

Other embodiments of formula (IA) are directed to any one of the embodiments described above for formula (IA) wherein R¹ is:

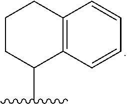

In another embodiment of the compounds of formula (IA) R¹ is alkyl substituted with one R²¹ group.

In another embodiment of the compounds of formula (IA) R¹ is alkyl substituted with one R²¹ group, and said alkyl is

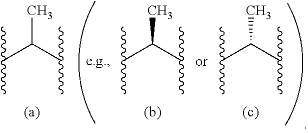

In another embodiment of the compounds of formula (IA) R¹ is alkyl (e.g., (a), (b) or (c) described above) substituted with one R²¹ group wherein said R²¹ group is aryl.

In another embodiment of the compounds of formula (IA) R¹ is alkyl (e.g., (a), (b) or (c) described above) substituted with one R²¹ group wherein said R²¹ group is phenyl.

In another embodiment of the compounds of formula (IA) R¹ is alkyl (e.g., (a), (b) or (c) described above) substituted with one R²¹ group wherein said R²¹ group is naphthyl.

In another embodiment of the compounds of formula (IA) R¹ is alkyl substituted with one R²¹ group, and said R²¹ group is substituted with two independently selected R²² groups.

In another embodiment of the compounds of formula (IA) R¹ is alkyl substituted with one R²¹ group, and said R²¹ group is substituted with one R²² group.

In another embodiment of the compounds of formula (IA) R¹ is alkyl substituted with one R²¹ group, wherein said alkyl group is (a) (e.g., (b) or (c)), as described above, and said R²¹ group is substituted with two independently selected R²² groups.

In another embodiment of the compounds of formula (IA) R¹ is alkyl substituted with one R²¹ group, wherein said alkyl group is (a) (e.g., (b) or (c)), as described above, and said R²¹ group is substituted with one R²² group.

In another embodiment of the compounds of formula (IA) R¹ is alkyl substituted with one R²¹ group, wherein said R²¹ group is aryl, and said R²¹ group is substituted with two independently selected R²² groups.

In another embodiment of the compounds of formula (IA) R¹ is alkyl substituted with one R²¹ group, wherein said R²¹ group is aryl, and said R²¹ group is substituted with one R²² group.

In another embodiment of the compounds of formula (IA) R¹ is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of the compounds of formula (IA) R¹ is alkyl substituted with one R²¹ group, wherein said R²¹ group is aryl, wherein said alkyl group is (a) (e.g., (b) or (c)), as described above, and said R²¹ group is substituted with one R²² group.

In another embodiment of the compounds of formula (IA) R¹ is alkyl substituted with one R²¹ group, wherein said R²¹ group is aryl, said R²¹ group is substituted with two independently selected R²² groups, and each R²² is halo.

In another embodiment of the compounds of formula (IA) R¹ is alkyl substituted with one R²¹ group, wherein said R²¹ group is aryl, and said R²¹ group is substituted with one R²² group, and said R²² is halo.

In another embodiment of the compounds of formula (IA) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of the compounds of formula (IA) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is halo.

In another embodiment of the compounds of formula (IA) R¹ is alkyl substituted with one R²¹ group, wherein said R²¹ group is aryl, said R²¹ group is substituted with two independently selected R²² groups, and each R²² is F.

In another embodiment of the compounds of formula (IA) R¹ is alkyl substituted with one R²¹ group, wherein said R²¹ group is aryl, and said R²¹ group is substituted with one R²² group, and said R²² is F.

In another embodiment of the compounds of formula (IA) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of the compounds of formula (IA) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of the compounds of formula (IA) R¹ is:

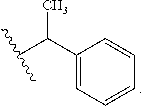

In another embodiment of the compounds of formula (IA) R¹ is:

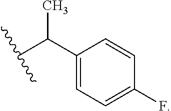

In another embodiment of the compounds of formula (IA) R¹ is:

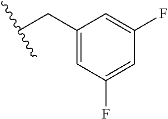

In another embodiment of the compounds of formula (IB) W is —S(O)—.

In another embodiment of the compounds of formula (IB) W is —S(O)₂—.

In another embodiment of the compounds of formula (IB) R⁸ is H.

In another embodiment of the compounds of formula (IB) R⁸ is H, and W is —S(O)—.

In another embodiment of the compounds of formula (IB) R⁸ is H, and W is —S(O)₂—.

In another embodiment of the compounds of formula (IB) R¹⁰ is aryl.

In another embodiment of the compounds of formula (IB) R¹⁰ is aryl, R⁸ is H, and W is —S(O)—.

In another embodiment of the compounds of formula (IB) R¹⁰ is aryl, R⁸ is H, and W is —S(O)₂—.

In another embodiment of the compounds of formula (IB) R¹⁰ is phenyl.

In another embodiment of the compounds of formula (IB) R¹⁰ is phenyl, R⁸ is H, and W is —S(O)—.

In another embodiment of the compounds of formula (IB) R¹⁰ is aryl substituted with one or more independently selected (e.g., one) R²¹ groups.

In another embodiment of the compounds of formula (IB) R¹⁰ is phenyl substituted with one or more (e.g., one) independently selected R²¹ groups.

In another embodiment of the compounds of formula (IB) R¹⁰ is aryl substituted with one or more (e.g., one) independently selected R²¹ groups, and R⁸ is H, and W is —S(O)—.

In another embodiment of the compounds of formula (IB) R¹⁰ is phenyl substituted with one or more (e.g., one) independently selected R²¹ groups, and R⁸ is H, and W is —S(O)—.

In another embodiment of the compounds of formula (IB) R¹⁰ is phenyl substituted with one or more (e.g., one) independently selected R²¹ groups and R⁸ is H, and W is —S(O)₂—.

In another embodiment of the compounds of formula (IB) R¹⁰ is aryl substituted with one R²¹ group.

In another embodiment of the compounds of formula (IB) R¹⁰ is aryl substituted with one R²¹ group, R⁸ is H, and W is —S(O)—.

In another embodiment of the compounds of formula (IB) R¹⁰ is aryl substituted with one R²¹ group, R⁸ is H, and W is —S(O)₂—.

In another embodiment of the compounds of formula (IB) R¹⁰ is phenyl substituted with one R²¹ group.

In another embodiment of the compounds of formula (IB) R¹⁰ is phenyl substituted with one R²¹ group, R⁸ is H, and W is —S(O)—.

In another embodiment of the compounds of formula (IB) R¹⁰ is phenyl substituted with one R²¹ group, R⁸ is H, and W is —S(O)₂—.

In another embodiment of the compounds of formula (IB) R¹⁰ is aryl substituted with one R²¹ group, wherein said R²¹ group is —OR¹⁵. In one example, R¹⁵ is alkyl. In another example R¹⁵ is methyl.

In another embodiment of the compounds of formula (IB) R¹⁰ is aryl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)—.

In another embodiment of the compounds of formula (IB) R¹⁰ is aryl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)₂—.

In another embodiment of the compounds of formula (IB) R¹⁰ is phenyl substituted with one R²¹ group, wherein said R²¹ group is —OR¹⁵. In one example, R¹⁵ is alkyl. In another example R¹⁵ is methyl.

In another embodiment of the compounds of formula (IB) R¹⁰ is phenyl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)—.

In another embodiment of the compounds of formula (IB) R¹⁰ is phenyl substituted with one R²¹ group, wherein said R²¹ group is —OR¹⁵. In one example, R¹⁵ is alkyl. In another example R¹⁵ is methyl. In any one of these embodiments R⁸ is H, and W is —S(O)₂—.

In another embodiment of the compounds of formula (IB) R⁹ is heteroaryl.

In another embodiment of the compounds of formula (IB) R⁹ is heteroaryl, R⁸ is H, and W is —S(O)—.

In another embodiment of the compounds of formula (IB) R⁹ is heteroaryl, R⁸ is H, and W is —S(O)₂—.

In another embodiment of the compounds of formula (IB) R⁹ is heteroaryl substituted with one or more (e.g., one) independently selected R²¹ groups.

In another embodiment of the compounds of formula (IB) R⁹ is heteroaryl substituted with one or more (e.g., one) independently selected R²¹ groups, R⁸ is H, and W is —S(O)—.

In another embodiment of the compounds of formula (IB) R⁹ is heteroaryl substituted with one or more (e.g., one) independently selected R²¹ groups, R⁸ is H, and W is —S(O)₂—.

In another embodiment of the compounds of formula (IB) R⁹ is heteroaryl substituted with one or more (e.g., one) independently selected R²¹ groups, wherein each R²¹ group is the same or different alkyl group (e.g., methyl).

In another embodiment of the compounds of formula (IB) R⁹ is heteroaryl substituted with one or more (e.g., one) independently selected R²¹ groups, wherein each R²¹ group is the same or different alkyl group (e.g., methyl), R⁸ is H, and W is —S(O)—.

In another embodiment of the compounds of formula (IB) R⁹ is heteroaryl substituted with one or more (e.g., one) independently selected R²¹ groups, wherein each R²¹ group is the same or different alkyl group (e.g., methyl), R⁸ is H, and W is —S(O)₂—.

In another embodiment of the compounds of formula (IB) R⁹ is heteroaryl substituted with one R²¹ group.

In another embodiment of the compounds of formula (IB) R⁹ is heteroaryl substituted with one R²¹ group, R⁸ is H, and W is —S(O)—.

In another embodiment of the compounds of formula (IB) R⁹ is heteroaryl substituted with one R²¹ group, R⁸ is H, and W is —S(O)₂—.

In another embodiment of the compounds of formula (IB) R⁹ is heteroaryl substituted with one R²¹ group, wherein R²¹ is an alkyl group (e.g., methyl).

In another embodiment of the compounds of formula (IB) R⁹ is heteroaryl substituted with R²¹ group, wherein R²¹ is an alkyl group (e.g., methyl), R⁸ is H, and W is —S(O)—.

In another embodiment of the compounds of formula (IB) R⁹ is heteroaryl substituted with one R²¹ group, wherein R²¹ is an alkyl group (e.g., methyl), R⁸ is H, and W is —S(O)₂—.

In another embodiment of the compounds of formula (IB) R⁹ is imidazolyl.

In another embodiment of the compounds of formula (IA) R⁹ is imidazolyl, R⁸ is H, and W is —S(O)—.

In another embodiment of the compounds of formula (IB) R⁹ is imidazolyl, R⁸ is H, and W is —S(O)₂—.

In another embodiment of the compounds of formula (IB) R⁹ is imidazolyl substituted with one or more (e.g., one) independently selected R²¹ groups.

In another embodiment of the compounds of formula (IB) R⁹ is imidazolyl substituted with one or more (e.g., one) independently selected R²¹ groups, R⁸ is H; and W is —S(O)—.

In another embodiment of the compounds of formula (IB) R⁹ is imidazolyl substituted with one or more (e.g., one) independently selected R²¹ groups, R⁸ is H, and W is —S(O)₂—.

In another embodiment of the compounds of formula (IB) R⁹ is imidazolyl substituted with one or more (e.g., one) independently selected R²¹ groups, wherein each R²¹ group is the same or different alkyl group (e.g., methyl).

In another embodiment of the compounds of formula (IB) R⁹ is imidazolyl substituted with one or more (e.g., one) independently selected R²¹ groups, wherein each R²¹ group is the same or different alkyl group (e.g., methyl), R⁸ is H, and W is —S(O)—.

In another embodiment of the compounds of formula (IB) R⁹ is imidazolyl substituted with one or more (e.g., one) independently selected R²¹ groups, wherein each R²¹ group is the same or different alkyl group (e.g., methyl), R⁸ is H, and W is —S(O)₂—.

In another embodiment of the compounds of formula (IB) R⁹ is imidazolyl substituted with one R²¹ group.

In another embodiment of the compounds of formula (IB) R⁹ is imidazolyl substituted with one R²¹ group, R⁸ is H, and W is —S(O)—.

In another embodiment of the compounds of formula (IB) R⁹ is imidazolyl substituted with one R²¹ group, R⁸ is H, and W is —S(O)₂—.

In another embodiment of the compounds of formula (IB) R⁹ is imidazolyl substituted with one R²¹ group, wherein R²¹ is an alkyl group (e.g., methyl).

In another embodiment of the compounds of formula (IB) R⁹ is imidazolyl substituted with R²¹ group, wherein R²¹ is an alkyl group (e.g., methyl), R⁸ is H, and W is —S(O)—.

In another embodiment of the compounds of formula (IB) R⁹ is imidazolyl substituted with one R²¹ group, wherein R²¹ is an alkyl group (e.g., methyl), R⁸ is H, and W is —S(O)₂—.

In another embodiment of the compounds of formula (IB) R⁹ is heteroaryl, optionally substituted with one or more R²¹ groups, and R¹⁰ is aryl optionally substituted with one or more (e.g., one) R²¹ groups.

In another embodiment of the compounds of formula (IB) R⁹ is heteroaryl, optionally substituted with one R²¹ group, and R¹⁰ is aryl optionally substituted with one R²¹ group.

In another embodiment of the compounds of formula (IB) R⁹ is heteroaryl, optionally substituted with one or more R²¹ groups, and R¹⁰ is phenyl optionally substituted with one or more (e.g., one) R²¹ groups.

In another embodiment of the compounds of formula (IB) R⁹ is heteroaryl, optionally substituted with one R²¹ group, and R¹⁰ is phenyl optionally substituted with one R²¹ group.

In another embodiment of the compounds of formula (IB) R⁹ is imidazolyl, optionally substituted with one or more R²¹ groups, and R¹⁰ is aryl optionally substituted with one or more (e.g., one) R²¹ groups.

In another embodiment of the compounds of formula (IB) R⁹ is imidazolyl, optionally substituted with one R²¹ group, and R¹⁰ is aryl optionally substituted with one R²¹ group.

In another embodiment of the compounds of formula (IB) R⁹ is imidazolyl, optionally substituted with one or more R²¹ groups, and R¹⁰ is phenyl optionally substituted with one or more (e.g., one) R²¹ groups.

In another embodiment of the compounds of formula (IB) R⁹ is imidazolyl, optionally substituted with one R²¹ group, and R¹⁰ is phenyl optionally substituted with one R²¹ group.

In another embodiment of the compounds of formula (IB) R⁹ is heteroaryl, optionally substituted with one or more R²¹ groups, R¹⁰ is aryl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is heteroaryl, optionally substituted with one R²¹ group, and R¹⁰ is aryl optionally substituted with one R²¹ group, R⁸ is H, and W is —S(O)—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is heteroaryl, optionally substituted with one or more R²¹ groups, and R¹⁰ is phenyl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is heteroaryl, optionally substituted with one R²¹ group, and R¹⁰ is phenyl optionally substituted with one R²¹ group, R⁸ is H, and W is —S(O)—. In one example the R²¹ group for R⁹ is independently selected from alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one or more R²¹ groups, and R¹⁰ is aryl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one R²¹ group, and R¹⁰ is aryl optionally substituted with one R²¹ group, R⁸ is H, and W is —S(O)—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one or more R²¹ groups, and R¹⁰ is phenyl optionally substituted with one or more (e.g., one) R²¹ groups, R⁸ is H, and W is —S(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one R²¹ group, and R¹⁰ is phenyl optionally substituted with one R²¹ group, R⁸ is H, and W is —S(O)—. In one example the R²¹ group for R⁹ is independently selected from alkyl. In another example of this embodiment the R²¹ group for R¹⁰ is independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is heteroaryl, optionally substituted with one or more R²¹ groups, R¹⁰ is aryl optionally substituted with one or more (e.g., one) R²¹ groups, R⁸ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is heteroaryl, optionally substituted with one R²¹ group, and R¹⁰ is aryl optionally substituted with one R²¹ group, R⁸ is H, and W is —S(O)₂—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is heteroaryl, optionally substituted with one or more R²¹ groups, and R¹⁰ is phenyl optionally substituted with one or more (e.g., one) R²¹ groups, R⁸ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is heteroaryl, optionally substituted with one R²¹ group, and R¹⁰ is phenyl optionally substituted with one R²¹ group, R⁸ is H, and W is —S(O)₂—. In one example the R²¹ group for R⁹ is independently selected from alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one or more R²¹ groups, and R¹⁰ is aryl optionally substituted with one or more (e.g., one) R²¹ groups, R⁸ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one R²¹ group, and R¹⁰ is aryl optionally substituted with one R²¹ group, R⁸ is H, and W is —S(O)₂—. In one example the R²¹ group for R⁹ is alkyl. In another example of this embodiment the R²¹ group for R¹⁰ is —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one or more R²¹ groups, and R¹⁰ is phenyl optionally substituted with one or more (e.g., one) R²¹ groups, R⁸ is H, and W is —S(O)₂—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of the compounds of formula (IB) R⁹ is imidazolyl, optionally substituted with one R²¹ group, and R¹⁰ is phenyl optionally substituted with one R²¹ group, R⁸ is H, and W is —S(O)₂—. In one example the R²¹ group for R⁹ is independently selected from alkyl. In another example of this embodiment the R²¹ group for R¹⁰ is independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

Other embodiments of the compounds of formula (IB) are directed to any one of the above embodiments for formula (IA) wherein R⁹ is:

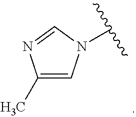

Other embodiments of the compounds of formula (IB) are directed to any one of the above embodiments for the compounds of formula (IB) wherein R¹⁰ is:

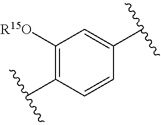

(wherein the —OR¹⁵ is ortho to the carbon to which R⁹ is bound to, i.e., the R⁹—R¹⁰— moiety is:

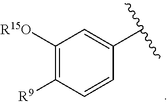

Other embodiments for the compounds of formula (IB) are directed to any one of the above embodiments for formula (IA) wherein R¹⁰ is:

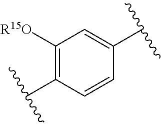

(wherein the —OCH₃ is ortho to the carbon to which R⁹ is bound to, i.e., the R⁹—R¹⁰— moiety is:

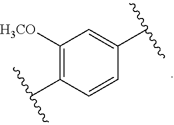

In another embodiment of the compounds of formula (IB) R¹ is benzofusedcycloalkyl.

In another embodiment of the compounds of formula (IB) R¹ is:

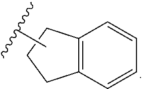

In another embodiment of the compounds of formula (IB) R¹ is:

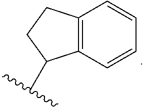

In another embodiment of the compounds of formula (IB) R¹ is:

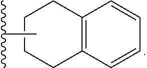

In another embodiment of the compounds of formula (IB) R¹ is:

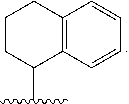

Other embodiments of the compounds of formula (IB) are directed to any one of the embodiments described above for formula (IB) wherein R¹ is benzofusedcycloalkyl.

Other embodiments of the compounds of formula (IB) are directed to any one of the embodiments described above for formula (IB) wherein R¹ is:

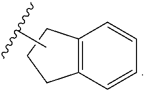

Other embodiments of formula (IB) are directed to any one of the embodiments described above for formula (IB) wherein R¹ is:

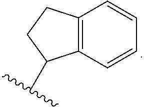

Other embodiments of formula (IB) are directed to any one of the embodiments described above for formula (IB) wherein R¹ is:

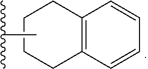

Other embodiments of formula (IB) are directed to any one of the embodiments described above for formula (IB) wherein R¹ is:

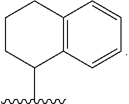

In another embodiment of the compounds of formula (IB) R¹ is alkyl substituted with one R²¹ group.

In another embodiment of the compounds of formula (IB) R¹ is alkyl substituted with one R²¹ group, and said alkyl is

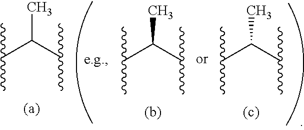

In another embodiment of the compounds of formula (IB) R¹ is alkyl (e.g., (a), (b) or (c) described above) substituted with one R²¹ group wherein said R²¹ group is aryl.

In another embodiment of the compounds of formula (IB) R¹ is alkyl (e.g., (a), (b) or (c) described above) substituted with one R²¹ group wherein said R²¹ group is phenyl.

In another embodiment of the compounds of formula (IB) R¹ is alkyl (e.g., (a), (b) or (c) described above) substituted with one R²¹ group wherein said R²¹ group is naphthyl.

In another embodiment of the compounds of formula (IB) R¹ is alkyl substituted with one R²¹ group, and said R²¹ group is substituted with two independently selected R²² groups.

In another embodiment of the compounds of formula (IB) R¹ is alkyl substituted with one R²¹ group, and said R²¹ group is substituted with one R²² group.

In another embodiment of the compounds of formula (IB) R¹ is alkyl substituted with one R²¹ group, wherein said alkyl group is (a) (e.g., (b) or (c)), as described above, and said R²¹ group is substituted with two independently selected R²² groups.

In another embodiment of the compounds of formula (IB) R¹ is alkyl substituted with one R²¹ group, wherein said alkyl group is (a) (e.g., (b) or (c)), as described above, and said R²¹ group is substituted with one R²² group.

In another embodiment of the compounds of formula (IB) R¹ is alkyl substituted with one R²¹ group, wherein said R²¹ group is aryl, and said R²¹ group is substituted with two independently selected R²² groups.

In another embodiment of the compounds of formula (IB) R¹ is alkyl substituted with one R²¹ group, wherein said R²¹ group is aryl, and said R²¹ group is substituted with one R²² group.

In another embodiment of the compounds of formula (IB) R¹ is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of the compounds of formula (IB) R¹ is alkyl substituted with one R²¹ group, wherein said R²¹ group is aryl, wherein said alkyl group is (a) (e.g., (b) or (c)), as described above, and said R²¹ group is substituted with one R²² group.

In another embodiment of the compounds of formula (IB) R¹ is alkyl substituted with one R²¹ group, wherein said R²¹ group is aryl, said R²¹ group is substituted with two independently selected R²² groups, and each R²² is halo.

In another embodiment of the compounds of formula (IB) R¹ is alkyl substituted with one R²¹ group, wherein said R²¹ group is aryl, and said R²¹ group is substituted with one R²² group, and said R²² is halo.

In another embodiment of the compounds of formula (IB) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of the compounds of formula (IB) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is halo.

In another embodiment of the compounds of formula (IB) R¹ is alkyl substituted with one R²¹ group, wherein said R²¹ group is aryl, said R²¹ group is substituted with two independently selected R²² groups, and each R²² is F.

In another embodiment of the compounds of formula (IB) R¹ is alkyl substituted with one R²¹ group, wherein said R²¹ group is aryl, and said R²¹ group is substituted with one R²² group, and said R²² is F.

In another embodiment of the compounds of formula (IB) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of the compounds of formula (IB) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of the compounds of formula (IB) R¹ is:

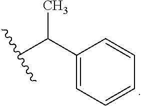

In another embodiment of the compounds of formula (IB) R¹ is:

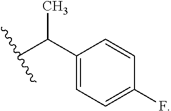

In another embodiment of the compounds of formula (IB) R¹ is:

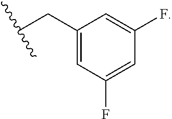

Other embodiments of this invention are directed to any one of the above embodiments directed to compounds of formula (IA) or (IB) wherein a compound of the formula (IA.1), (IB.1), (IC), (IC.1), (ID), (ID.1), (ID.2), (ID.3), (ID.4), (IE), (IE.1), (IF), (IF.1), (IG), (IG.1), (IG.2), (IH), (IJ), (IK), (IL) or (IM) is used instead of (IA) or (IB).

Representative compounds of formula (I) having formula (IA) include but are not limited to:

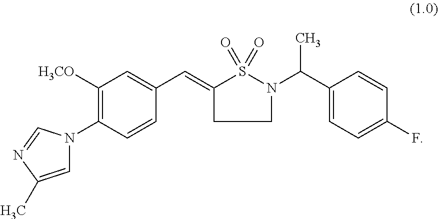

Representative compounds of formula (I) having formula (IB) include but are not limited to:

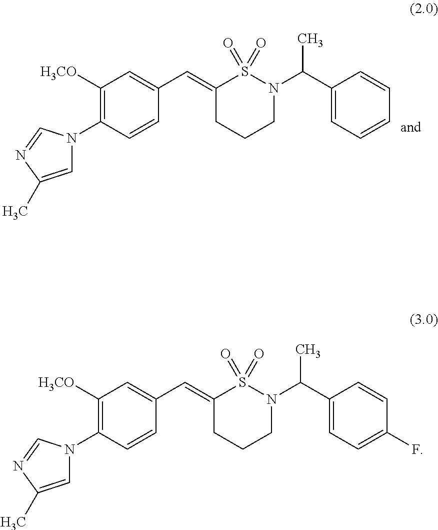

Representative compounds of formula (I) wherein R² and R³ are not taken together to form a ring, include, but are not limited to, the compounds in Table 1 below.

TABLE 1
(4.0)

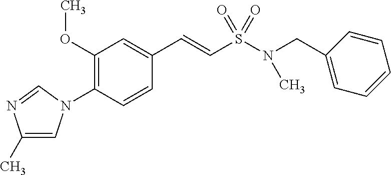

(5.0)

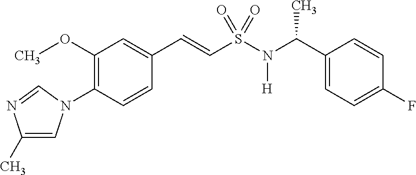

(6.0)

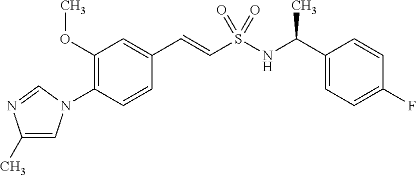

(7.0)

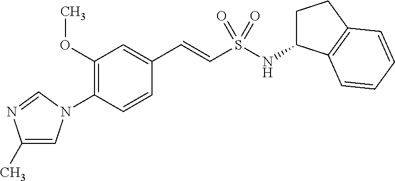

(8.0)

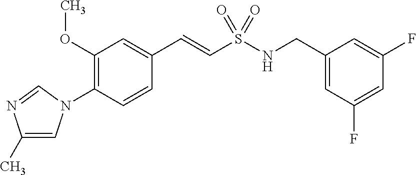

(9.0)

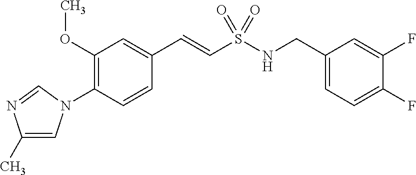

(10.0)

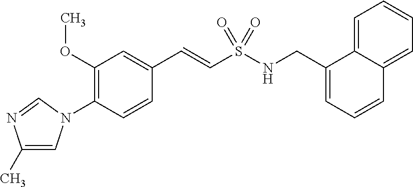

(11.0)

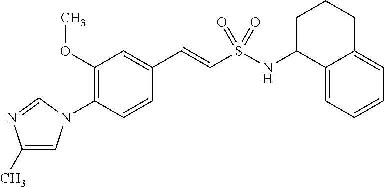

(12.0)

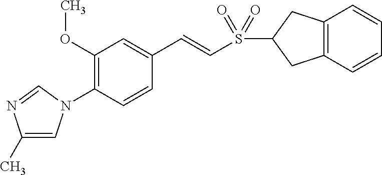

(13.0)

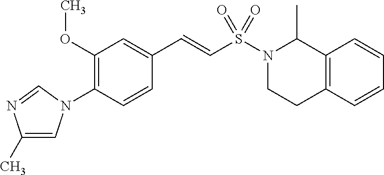

Representative compounds of formula (I) having formula (IB.1) include but are not limited to:

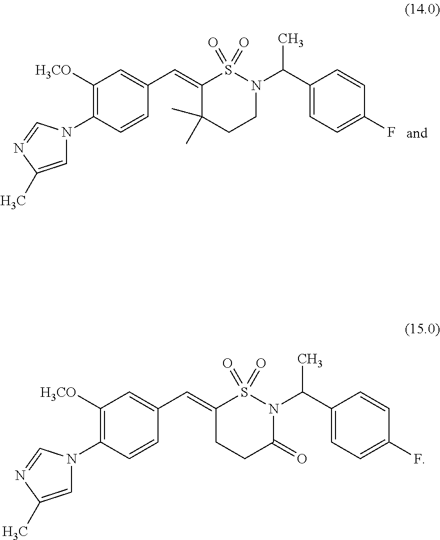

Representative compounds of formula (I) having formula (IC) include but are not limited to:

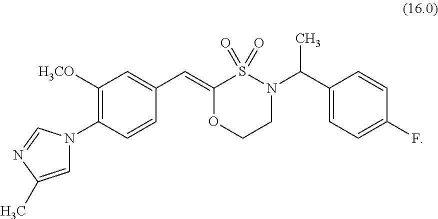

Representative compounds of formula (I) having formula (IL) include but are not limited to:

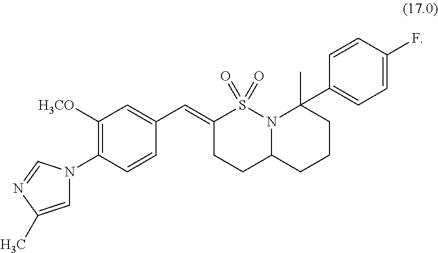

Thus another embodiment of this invention is directed to a compound of formula 1.0.

Another embodiment of this invention is directed to a compound of formula 2.0.

Another embodiment of this invention is directed to a compound of formula 3.0.

Another embodiment of this invention is directed to a compound of formula 4.0.

Another embodiment of this invention is directed to a compound of formula 5.0.

Another embodiment of this invention is directed to a compound of formula 6.0.

Another embodiment of this invention is directed to a compound of formula 7.0.

Another embodiment of this invention is directed to a compound of formula 8.0.

Another embodiment of this invention is directed to a compound of formula 9.0.

Another embodiment of this invention is directed to a compound of formula 10.0.

Another embodiment of this invention is directed to a compound of formula 11.0.

Another embodiment of this invention is directed to a compound of formula 12.0.

Another embodiment of this invention is directed to a compound of formula 13.0.

Another embodiment of this invention is directed to a compound of formula 14.0.

Another embodiment of this invention is directed to a compound of formula 15.0.

Another embodiment of this invention is directed to a compound of formula 161.0.

Another embodiment of this invention is directed to a compound of formula 17.0.

Another embodiment of this invention is directed to a pharmaceutically acceptable salt of a compound of formula 1.0.

Another embodiment of this invention is directed to a pharmaceutically acceptable salt of a compound of formula 2.0.

Another embodiment of this invention is directed to a pharmaceutically acceptable salt of a compound of formula 3.0.

Another embodiment of this invention is directed to a pharmaceutically acceptable salt of a compound of formula 4.0.

Another embodiment of this invention is directed to a pharmaceutically acceptable salt of a compound of formula 5.0.

Another embodiment of this invention is directed to a pharmaceutically acceptable salt of a compound of formula 6.0.

Another embodiment of this invention is directed to a pharmaceutically acceptable salt of a compound of formula 7.0.

Another embodiment of this invention is directed to a pharmaceutically acceptable salt of a compound of formula 8.0.

Another embodiment of this invention is directed to a pharmaceutically acceptable salt of a compound of formula 9.0.

Another embodiment of this invention is directed to a pharmaceutically acceptable salt of a compound of formula 10.0.

Another embodiment of this invention is directed to a pharmaceutically acceptable salt of a compound of formula 11.0.

Another embodiment of this invention is directed to a pharmaceutically acceptable salt of a compound of formula 12.0.

Another embodiment of this invention is directed to a pharmaceutically acceptable salt of a compound of formula 13.0.

Another embodiment of this invention is directed to a pharmaceutically acceptable salt of a compound of formula 14.0.

Another embodiment of this invention is directed to a pharmaceutically acceptable salt of a compound of formula 15.0.

Another embodiment of this invention is directed to a pharmaceutically acceptable salt of a compound of formula 16.0.

Another embodiment of this invention is directed to a pharmaceutically acceptable salt of a compound of formula 17.0.

Another embodiment of this invention is directed to a pharmaceutically acceptable ester of a compound of formula 1.0.

Another embodiment of this invention is directed to a pharmaceutically acceptable ester of a compound of formula 2.0.

Another embodiment of this invention is directed to a pharmaceutically acceptable ester of a compound of formula 3.0.

Another embodiment of this invention is directed to a pharmaceutically acceptable ester of a compound of formula 4.0.

Another embodiment of this invention is directed to a pharmaceutically acceptable ester of a compound of formula 5.0.

Another embodiment of this invention is directed to a pharmaceutically acceptable ester of a compound of formula 6.0.

Another embodiment of this invention is directed to a pharmaceutically acceptable ester of a compound of formula 7.0.

Another embodiment of this invention is directed to a pharmaceutically acceptable ester of a compound of formula 8.0.

Another embodiment of this invention is directed to a pharmaceutically acceptable ester of a compound of formula 9.0.

Another embodiment of this invention is directed to a pharmaceutically acceptable ester of a compound of formula 10.0.

Another embodiment of this invention is directed to a pharmaceutically acceptable ester of a compound of formula 11.0.

Another embodiment of this invention is directed to a pharmaceutically acceptable ester of a compound of formula 12.0.

Another embodiment of this invention is directed to a pharmaceutically acceptable ester of a compound of formula 13.0.

Another embodiment of this invention is directed to a pharmaceutically acceptable ester of a compound of formula 14.0.

Another embodiment of this invention is directed to a pharmaceutically acceptable ester of a compound of formula 15.0.

Another embodiment of this invention is directed to a pharmaceutically acceptable ester of a compound of formula 16.0.

Another embodiment of this invention is directed to a pharmaceutically acceptable ester of a compound of formula 17.0.

Another embodiment of this invention is directed to a solvate of a compound of formula 1.0.

Another embodiment of this invention is directed to a solvate of a compound of formula 2.0.

Another embodiment of this invention is directed to a solvate of a compound of formula 3.0.

Another embodiment of this invention is directed to a solvate of a compound of formula 4.0.

Another embodiment of this invention is directed to a solvate of a compound of formula 5.0.

Another embodiment of this invention is directed to a solvate of a compound of formula 6.0.

Another embodiment of this invention is directed to a solvate of a compound of formula 7.0.

Another embodiment of this invention is directed to a solvate of a compound of formula 8.0.

Another embodiment of this invention is directed to a solvate of a compound of formula 9.0.

Another embodiment of this invention is directed to a solvate of a compound of formula 10.0.

Another embodiment of this invention is directed to a solvate of a compound of formula 11.0.

Another embodiment of this invention is directed to a pharmaceutically acceptable ester of a compound of formula 12.0.

Another embodiment of this invention is directed to a pharmaceutically acceptable ester of a compound of formula 13.0.

Another embodiment of this invention is directed to a pharmaceutically acceptable ester of a compound of formula 14.0.

Another embodiment of this invention is directed to a pharmaceutically acceptable ester of a compound of formula 15.0.

Another embodiment of this invention is directed to a pharmaceutically acceptable ester of a compound of formula 16.0.

Another embodiment of this invention is directed to a pharmaceutically acceptable ester of a compound of formula 17.0.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ antagonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

It is noted that the carbons of formula I and other formulas herein may be replaced with 1 to 3 silicon atoms so long as all valency requirements are satisfied.

As used above, and throughout this disclosure, the following terms, unless otherwise indicated, shall be understood to have the following meanings:

“Patient” includes both human and animals.

“Mammal” means humans and other mammalian animals.

“One or more” means that there is at least one and there can be more than one, and examples include 1, 2 or 3, or 1 and 2, or 1.

“At least one” means there is at least one and there can be more than one, and examples include 1, 2 or 3, or 1 and 2, or 1.

“Fused benzocycloalkyl ring” means a phenyl ring fused to a cycloalkyl ring (as cycloalkyl is defined below), such as, for example,

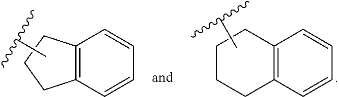

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or this before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)— cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

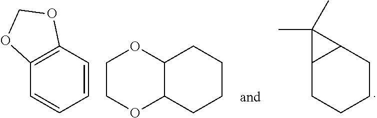

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes rings wherein ═O replaces two available hydrogens on the same carbon atom on a ring system (i.e., heterocyclyl includes rings having a carbonyl in the ring). An example of such moiety is pyrrolidone:

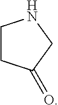

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom on a ring system (i.e., heterocyclyl includes rings having a carbonyl in the ring). An example of such moiety is pyrrolidinone:

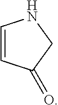

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

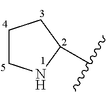

there is no —OH attached directly to carbons marked 2 and 5.

It should also be noted that tautomeric forms such as, for example, the moieties:

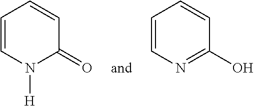

are considered equivalent in certain embodiments of this invention.

It should also be noted that tautomeric forms such as, for example, the moieties:

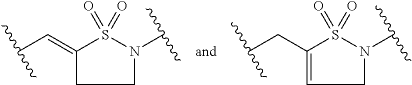

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I.R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phemethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula I, and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula I, and of the salts, solvates, esters and prodrugs of the compounds of Formula I, are intended to be included in the present invention.

The compounds according to the invention can have pharmacological properties; in particular, the compounds of Formula I can be modulators of gamma secretase (including inhibitors, antagonists and the like).

More specifically, the compounds of Formula I can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula I, or a pharmaceutically acceptable salt, solvate, ester or prodrug of said compound to the mammal.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula I, or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Yet another aspect of this invention is a kit comprising an amount of at least one compound of Formula I, or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative example which should not be construed to limit the scope of the disclosure.

Alternative mechanistic pathways and analogous structures will be apparent to those skilled in the art.

### EXAMPLES

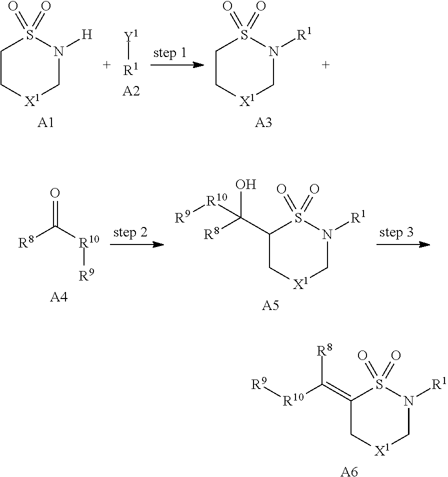

### Method A, Step 1

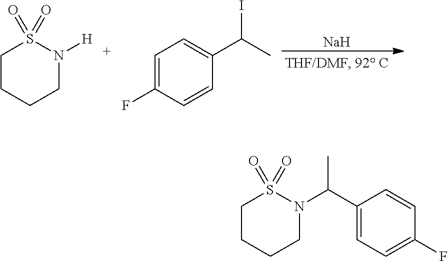

To a solution of compound A1 (X¹=CH₂, 1.08 g, 8.0 mmol) in THF/DMF (10/10 ml) was added NaH (60%, 352 mg, 8.8 mmol) and compound A2 (Y¹=I, R¹=1-(4-F-Phenyl)-ethyl, 2.0 g, 8.0 mmol), and the resulting mixture was heated at 92° C. overnight. Then it was diluted with H₂O, extracted with EtOAc, dried (Na₂SO₄), concentrated and purified by silica gel flash chromatography (Hex/EtOAc) to afford compound A3 as colorless oil (X¹=CH₂, R¹=1-(4-F-Phenyl)-ethyl, 1.2 g, 58%). MS (M+1): 258.

### Method A, Step 2

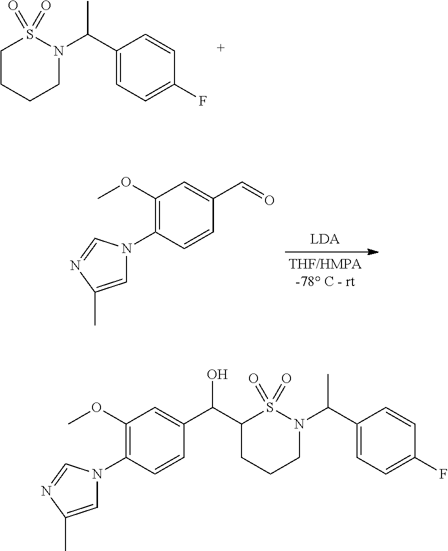

To a solution of compound A3 (X¹=CH₂, R¹=1-(4-F-Phenyl)-ethyl, 300 mg, 1.17 mmol) in THF/HMPA (3 ml/400 ul) was added LDA (2M, 0.7 ml, 1.40 mmol) at −78° C. under N₂ and stirred for 10 min. Then compound A4 (R⁸=H, R¹⁰=3-MeO-Phenyl, R⁹=4-(4-Methyl-imidazol-1-yl), 252 mg, 1.17 mmol) was added and the resulting mixture was stirred overnight. Then it was quenched with saturated aqueous NH₄Cl, extracted with EtOAc, dried (Na₂SO4), concentrated and purified by silica gel flash chromatography (EtOAc) to afford compound A5 as colorless oil (X¹=CH₂, R¹=1-(4-F-Phenyl)-ethyl, R⁸=H, R¹⁰=3-MeO-Phenyl, R⁹=4-(4-Methyl-imidazol-1-yl), 120 mg, 22%). MS (M+1): 474.

### Method A, Step 3

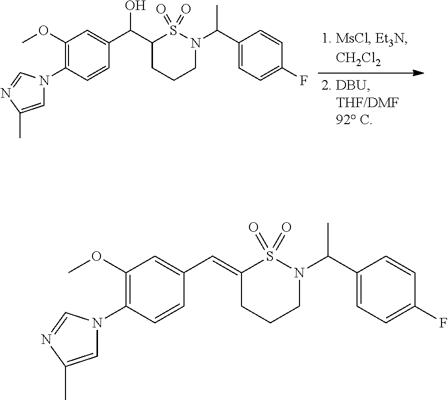

To a solution of compound A5 (X¹=CH₂, R¹=1-(4-F-Phenyl)-ethyl, R⁸=H, R¹⁰=3-MeO-Phenyl, R⁹=4-(4-Methyl-imidazol-1-yl), 123 mg, 0.26 mmol) in CH₂Cl₂ (2 ml) was added Et₃N (0.109 ml, 0.78 mmol) and MsCl (0.059 ml, 0.78 mmol). The resulting mixture was stirred overnight, then diluted with CH₂Cl₂, washed with H₂O, dried (Na₂SO₄), concentrated and purified by silica gel flash chromatography (CH₂Cl₂/MeOH) to afford product as colorless oil (130 mg, 90%). The above product was dissolved in THF/DMF (2/2 ml) and added with DBU (110 mg, 0.72 mmol) and heated at 92° C. for 4 hrs. Then it was concentrated and purified by silica gel flash chromatography (CH₂Cl₂:MeOH) to afford compound A6 as colorless oil (X¹=CH₂, R¹=1-(4-F-Phenyl)-ethyl, R⁸=H, R¹⁰=3-MeO-Phenyl, R⁹=4-(4-Methyl-imidazol-1-yl), 50 mg, 46% yield). MS (M+1): 456.

Following the procedures similar to those of method A, step 1-3, the compounds in Table 2 were prepared.

TABLE 2

| Compound | Obs. Mass |
|---|---|
| 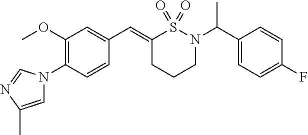 | 456.3 |
| 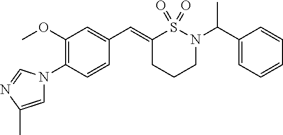 | 438.2 |
| 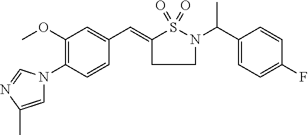 | 442.2 |

### Method B

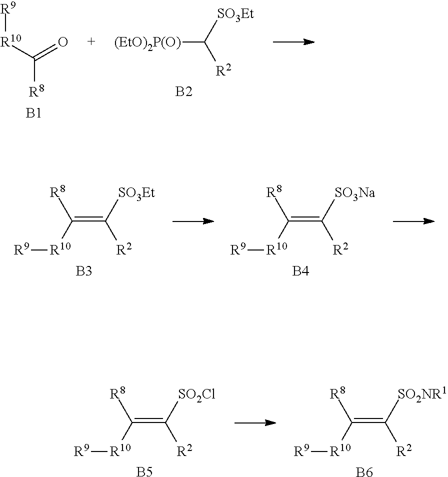

### Method B, Step 1

Compound B2 (R²=H, 1.5 g, 5.6 mmol, synthesized as in Tett. 1987, 43, 5125) in 40 ml of anhydrous THF at 0° C. was treated with NaH (0.25 g, 5.9 mmol, 60% in mineral oil). After 20 min, B1 (R⁸=H, R¹⁰=3-MeO-Phenyl, R⁹=4-(4-Methylimidazol-1-yl), 1.0 g, 4.62 mmol) was added and the reaction warmed to ambient temperature. After 18 h, the reaction mixture was quenched with saturated aqueous NH₄Cl and extracted with EtOAc (2×). The combined organic layers washed with saturated aqueous NaHCO₃, brine, dried over MgSO₄ and concentrated in vacuo. Flash chromatography (gradient 1→10% MeOH/CH₂Cl₂) provided 1.2 g (81%) of B3 (R⁸=H, R²=H, R¹⁰=3-MeO-Phenyl, R⁹=4-(4-Methyl-imidazol-1-yl)).

### Method B, Step 2

B3 (R⁸=H, R²=H, R¹⁰=3-MeO-Phenyl, R⁹=4-(4-Methyl-imidazol-1-yl), 1.2 g, 3.72 mmol) in 110 mL EtOH and 4 mL H₂O was heated to reflux. After 18 h, the reaction mixture was cooled to ambient temperature and NaOAc (0.34 g, 4.09 mmol) was added. After 1 additional h, the reaction mixture was concentrated in vacuo to afford B4 (R⁸=H, R²=H, R¹⁰=3-MeO-Phenyl, R⁹=4-(4-Methyl-imidazol-1-yl)) and used without further purification.

### Method B, Step 3

A solution of crude B4 (R⁸=H, R²=H, R¹⁰=3-MeO-Phenyl, R⁹=4-(4-Methyl-imidazol-1-yl)) from the previous step in CH₂Cl₂ (40 mL) was treated with N,N-DMF (400 μL) and phosgene (3.7 mL, 20% in toluene). After 2.5 h, the reaction mixture was concentrated in vacuo to provide B5 (R⁸=H, R²=H, R¹⁰=3-MeO-Phenyl, R⁹=4-(4-Methyl-imidazol-1-yl), 1.9 g) which was used without further purification.

### Method B, Step 4

A solution of crude B5 (150 mg, 0.48 mmol, R⁸=H, R²=H, R¹⁰=3-MeO-Phenyl, R⁹=4-(4-Methyl-imidazol-1-yl)) from the previous step in CH₂Cl₂ (2 mL) was treated with Et₃N (250 μL, 1.79 mmol) and 3,5-difluoro benzylamine (75 μL, 0.63 mmol). After 18 h, the reaction mixture was quenched with saturated aqueous NH₄Cl and extracted with EtOAc (2×). The combined organic layers washed with saturated aqueous NaHCO₃, brine, dried over MgSO₄ and concentrated in vacuo. Preparative thin layer chromatography (gradient 5% MeOH/CH₂Cl₂) provided 8 mg (4%) of B6 (R⁸=H, R²=H, R¹⁰=3-MeO-Phenyl, R⁹=4-(4-Methyl-imidazol-1-yl), R¹=3,5-difluorobenzyl): ¹H NMR (CDCl₃, 400 MHz) δ 7.76 (d, J=1.5 Hz, 1H), 7.44 (d, J=15.4 Hz, 1H), 7.30 (d, J=8.1 Hz, 1H), 7.13 (dd, J=8.1, 1.5 Hz, 1H), 7.06 (d, J=1.5 Hz, 1H), 6.95 (d, J=1.5 Hz, 1H), 6.92-6.89 (m, 2H), 6.74 (s, 1H), 6.71 (s, 1H), 5.20 (br s, 1H), 4.28 (s, 2H), 3.90 (s, 3H), 2.29 (s, 3H). MS (ES-LCMS, M+1): 420.2.

Following procedures similar to those of Method B, the compounds in Table 3 were prepared.

TABLE 3

| Compound | Obs. Mass |
|---|---|
| 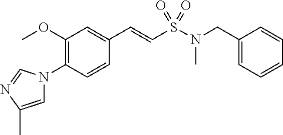 | 398.2 |
| 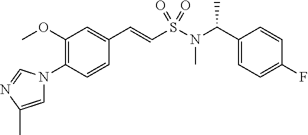 | 416.2 |
| 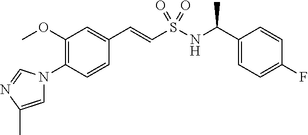 | 416.2 |
| 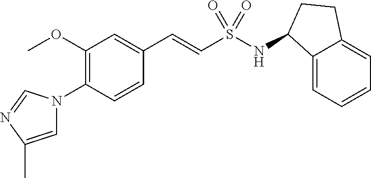 | 410.2 |
| 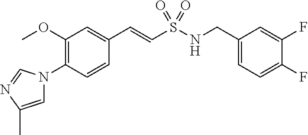 | 420.2 |
| 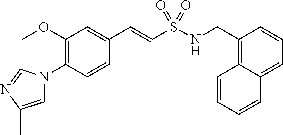 | 434.2 |
| 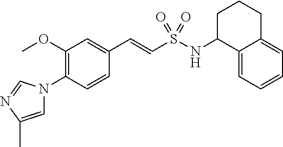 | 424.2 |

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-W02 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody W02 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated A□ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

The compounds in Table 4 below had a Cellular Aβ42 IC50 in the range of about 104 nM to about 19912 nM.

TABLE 4
Compound

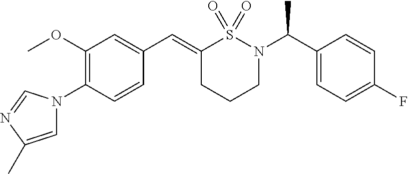

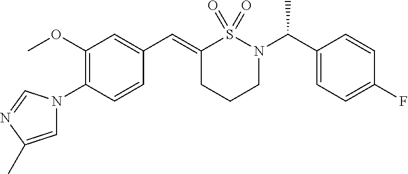

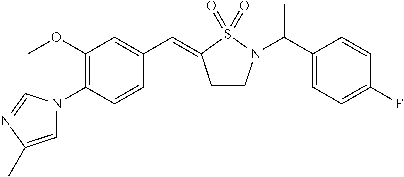

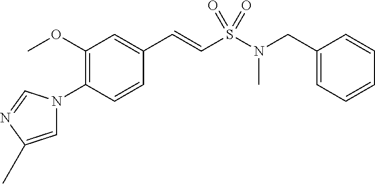

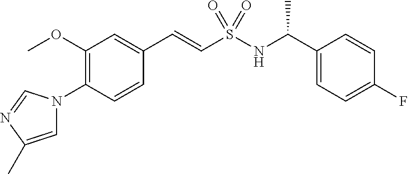

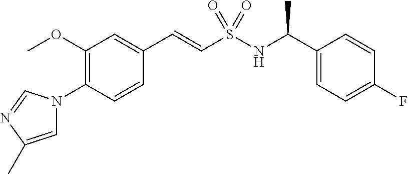

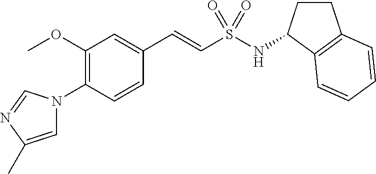

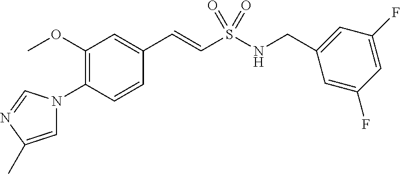

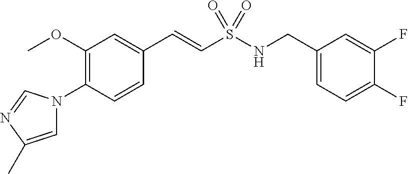

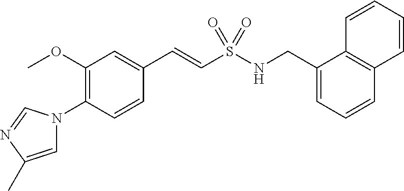

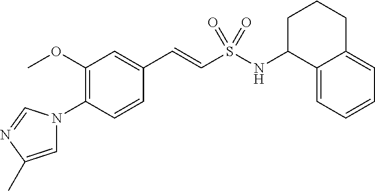

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound of the formula: 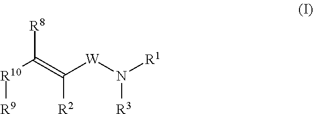
or the pharmaceutically acceptable salts thereof, wherein:
R¹, R², R³, R⁸, R⁹, R¹⁰, and W are independently selected;
W is selected from the group consisting of; —S(O)—, and —S(O)₂—;
R¹ is selected from the group consisting of H, alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedheterocycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl-R¹ groups is optionally substituted with 1-5 independently selected R²¹ groups;
R² and R³ are each independently selected from the group consisting of H, alkyl-, alkenyl-, alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl-, and heterocyclyalkyl-; wherein each of said alkyl-, alkenyl- and alkynyl-, aryl-, arylalkyl-, alkylaryl-, cycloalkyl-, cycloalkenyl, cycloalkylalkyl-, cycloalkenyl-, heteroaryl-, heteroarylalkyl-, heterocyclyl-, heterocyclenyl- and heterocyclyalkyl- R¹ groups is optionally substituted with 1-5 independently selected R²¹ groups; or
R² and R³ taken together, along with the atoms to which they are bound, form a ring selected from the group consisting of:
(a) a 5 to 6 membered heterocycloalkyl ring, said heterocycloalkyl ring optionally comprising, in addition to W and in addition to the N adjacent to W, at least one other heteroatom independently selected from the group consisting of: —O—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one other heteroatom independently selected from the group consisting of: —O—, —S(O)—, —S(O)₂, and —C(O)—,
wherein said ring is optionally substituted with 1-5 independently selected R²¹ groups; or
R² and R³ taken together along with the atoms to which they are bound, and R¹ and R³ are taken together along with the atoms to which they are bound, form the fused ring moiety: 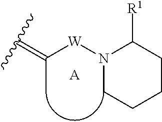
wherein Ring A is a ring selected from the group consisting of:
(a) a 5 to 6 membered heterocycloalkyl ring, said heterocycloalkyl ring optionally comprising, in addition to W and in addition to the N adjacent to W, at least one other heteroatom independently selected from the group consisting of: —O—, —NR¹⁴—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one other heteroatom independently selected from the group consisting of: —O—, —NR¹⁴—, —S(O)—, —S(O)₂, and —C(O)—, and
wherein said fused ring moiety is optionally substituted with 1-5 independently selected R²¹ groups; or
R¹ and R³ taken together with the atoms to which they are bound form a fused benzoheterocycloalkyl ring, and wherein said fused ring is optionally substituted with 1-5 independently selected R²¹ groups,
R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl-; wherein each of said R⁸ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl, heterocyclenyl- and heterocyclyalkyl- is optionally substituted with 1-3 independently selected R²¹ groups;
R⁹ is selected from the group consisting of: alkyl-, alkenyl-, alkynyl-, aryl-, arylalkyl-, alkylaryl-, cycloalkyl-, cycloalkenyl, cycloalkylalkyl, heteroaryl-, heteroarylalkyl-, heterocyclyl-, heterocyclenyl-, and heterocyclyalkyl-, wherein each of said R⁹ alkyl-, alkenyl- and alkynyl-, aryl-, arylalkyl-, alkylaryl-, cycloalkyl-, cycloalkenyl, cycloalkyl alkyl-, heteroaryl-, heteroarylalkyl-, heterocyclyl-, heterocyclenyl-, heterocyclyalkyl- and heterocyclyalkyl- is optionally substituted with 1-3 independently selected R²¹ groups;
R¹⁰ is selected from the group consisting of: a bond, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl-, heterocyclyalkenyl-, 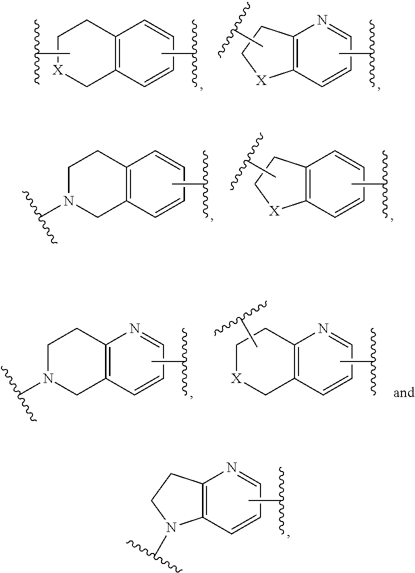
wherein X is selected from the group consisting of: O, —N(R¹⁴)— or —S—; and
wherein each of said R¹⁰ moieties is optionally substituted with 1-3 independently selected R²¹ groups;
R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —ON, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);
R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;
Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or
two R¹⁸ moieties on adjacent carbons can be linked together to form a 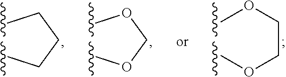
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl:
Each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —ON, —OR¹⁵, —C(O)R¹⁵, —C(O)OR¹⁵, —C(O)N(R¹⁵)(R¹⁶), —SR¹⁵, —S(O)N(R¹⁶)(R¹⁶), —CH(R¹⁵)(R¹⁶), —S(O)₂N(R¹⁶)(R¹⁶), —C(═NOR¹⁶)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —OH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups; and
Each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷); —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

2.-5. (canceled)

6. The compound of claim 1 wherein:
(a) R² is H and R³ is methyl, or R² is H and R³ is H;
(b) W is —S(O)₂;
c) R⁸ is H;
(d) R¹⁰ is aryl substituted with one R²¹ group, said R²¹ is —OR¹⁵, wherein said R¹⁵ is methyl;
(e) R⁹ is heteroaryl substituted with one R²¹ group, wherein said R²¹ is methyl, and said heteroaryl is imidazolyl.

7.-20. (canceled)

21. The compound of claim 1 wherein:
(a) R¹ is a fused benzocycloalkyl; or
(b) R¹ is selected from the group consisting of: 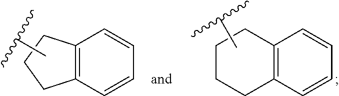
or
(c) R¹ selected from the group consisting of:

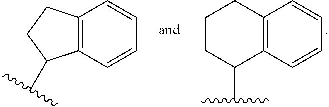

22.-23. (canceled)

24. The compound of claim 1 wherein:
(a) R¹ is alkyl substituted with one R²¹ group, and said R²¹ is selected from the group consisting of phenyl and naphthyl; or
(b) R¹ is alkyl substituted with one R²¹ group, said R²¹ group is aryl substituted with one or two independently selected R²² groups, wherein said aryl is phenyl, and said R²² group is halo.

25.-30. (canceled)

31. The compound of claim 24 wherein said halo is F in (b).

32. The compound of claim 1 wherein said R¹ is selected from the group consisting of:

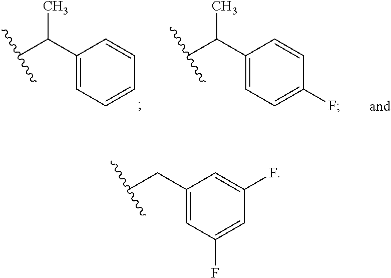

33.-34. (canceled)

35. The compound of claim 1 wherein: R² is H, R³ is selected from the group consisting of H and alkyl, W is —S(O)₂, R⁸ is H, R¹⁰ is aryl substituted with one R²¹ group, R⁹ is heteroaryl substituted with one R²¹ group, and R¹ is selected from the group consisting of: benzofusedcycloalkyl, and alkyl substituted with one R²¹ group.

36. The compound of claim 35 wherein:
(a) said R²¹ group on said R¹⁰ moiety is —OR¹⁵, said R²¹ group on said R⁹ moiety is alkyl, and said R²¹ group on said R¹ group is aryl; or
(b) said R²¹ group on said R¹⁰ moiety is —OR¹⁵, said R²¹ group on said R⁹ moiety is alkyl, and said R²¹ group on said R¹ group is aryl substituted with one R²² group; or
(c) said R²¹ group on said R¹⁰ moiety is —OR¹⁵, said R¹⁵ is alkyl, said R²¹ group on said R⁹ moiety is alkyl, and said R²¹ group on said R¹ group is aryl; or
(d) said R²¹ group on said R¹⁰ moiety is —OR¹⁵, said R¹⁵ is alkyl, said R²¹ group on said R⁹ moiety is alkyl, and said R²¹ group on said R¹ group is aryl substituted with one R²² group; or
(e) said R²¹ group on said R¹⁰ moiety is —OR¹⁵, said R¹⁵ is alkyl, said R²¹ group on said R⁹ moiety is alkyl, said R²¹ group on said R¹ group is aryl, and the R⁹ heteroaryl moiety is imidazolyl; or
(f) said R²¹ group on said R¹⁰ moiety is —OR¹⁵, said R¹⁵ is alkyl, said R²¹ group on said R⁹ moiety is alkyl, said R²¹ group on said R¹ group is aryl substituted with one R²² group, and the R⁹ heteroaryl moiety is imidazolyl.

37.-41. (canceled)

42. The compound of claim 36: wherein R¹ in (e) and in (f) is selected from the group consisting of:

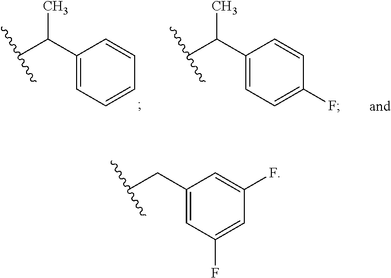

43.-47. (canceled)

48. The compound of claim 1 wherein R² and R³, taken together with the atoms to which they are bound, form a 5 to 6 membered ring.

49. The compound of claim 48 wherein:
(a) said ring is a five membered ring and the compound of formula (I) is a compound of formula (IA): 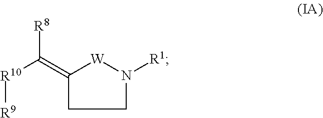
or
(b) said ring is a six membered ring and the compound of formula (I) is a compound of formula (1B):

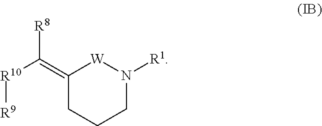

50. (canceled)

51. The compound of claim 48 wherein:
W is —S(O)₂;
(b) R⁸ is H;
(c) R¹⁰ is aryl substituted with one R²¹ group, said aryl is phenyl, said R²¹ group is —OR¹⁵, said R¹⁵ is methyl; and
(d) R⁹ is heteroaryl substituted with one R²¹ group, said R²¹ is methyl, said heteroaryl is imidazolyl.

52.-63. (canceled)

64. The compound of claim 48 wherein:
(a) R¹ is alkyl substituted with one R²¹ group, and said R²¹ is phenyl; or
(b) R¹ is alkyl substituted with one R²¹ group, and said R²¹ is phenyl substituted with one R²¹ group, and said R²¹ group is halo; or
(c) R¹ is alkyl substituted with one R²¹ group, and said R²¹ is phenyl substituted with one R²¹ group, and said R²¹ group is F; or
(d) R¹ is: 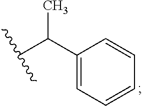
(e) R¹ is:

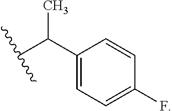

65.-72. (canceled)

73. The compound of claim 49 wherein:
(a) for the compound in (a), W is —S(O)₂, R⁸ is H, R¹⁰ is aryl substituted with one R²¹ group, R⁹ is heteroaryl substituted with one R²¹ group, and R¹ is alkyl substituted with one R²¹ group; or
(b) for the compound in (a), W is —S(O)₂, R⁸ is H, R¹⁰ is aryl substituted with one R²¹ group, R⁹ is heteroaryl substituted with one R²¹ group, R¹ is alkyl substituted with one R²¹ group, said R²¹ group on said R¹⁰ moiety is —OR¹⁵, said R²¹ group on said R⁹ moiety is alkyl, and said R²¹ group on said R¹ group is aryl; or
(c) for the compound in (a), W is —S(O)₂, R⁸ is H, R¹⁰ is aryl substituted with one R²¹ group, R⁹ is heteroaryl substituted with one R²¹ group, R¹ is alkyl substituted with one R²¹ group, wherein said R²¹ group on said R¹⁰ moiety is —OR¹⁵, said R²¹ group on said R⁹ moiety is alkyl, and said R²¹ group on said R¹ group is aryl substituted with one R²² group; or
(d) for the compound in (a), W is —S(O)₂, R⁸ is H, R¹⁰ is aryl substituted with one R²¹ group, R⁹ is heteroaryl substituted with one R²¹ group, R¹ is alkyl substituted with one R²¹ group, said R²¹ group on said R¹⁰ moiety is —OR¹⁵, said R¹⁵ is alkyl, said R²¹ group on said R⁹ moiety is alkyl, and said R²¹ group on said R¹ group is aryl; or
(e) for the compound in (a), W is —S(O)₂, R⁸ is H, R¹⁰ is aryl substituted with one R²¹ group, R⁹ is heteroaryl substituted with one R²¹ group, R¹ is alkyl substituted with one R²¹ group, wherein said R²¹ group on said R¹⁰ moiety is —OR¹⁵, said R¹⁵ is alkyl, said R²¹ group on said R⁹ moiety is alkyl, and said R²¹ group on said R¹ group is aryl substituted with one R²² group; or
(f) for the compound in (a), W is —S(O)₂, R⁸ is H, R¹⁰ is aryl substituted with one R²¹ group, R⁹ is heteroaryl substituted with one R²¹ group, said R⁹ is imidazolyl, R¹ is alkyl substituted with one R²¹ group, said R²¹ group on said R¹⁰ moiety is —OR¹⁵, said R¹⁵ is alkyl, said R²¹ group on said R⁹ moiety is alkyl, and said R²¹ group on said R¹ group is aryl; or
(g) for the compound in (a), W is —S(O)₂, R⁸ is H, R¹⁰ is aryl substituted with one R²¹ group, R⁹ is heteroaryl substituted with one R²¹ group, said R⁹ is imidazolyl, R¹ is alkyl substituted with one R²¹ group, wherein said R²¹ group on said R¹⁰ moiety is —OR¹⁵, said R¹⁵ is alkyl, said R²¹ group on said R⁹ moiety is alkyl, and said R²¹ group on said R¹ group is aryl substituted with one R²² group; or
(h) for the compound in (a), W is —S(O)₂, R⁸ is H, R¹⁰ is aryl substituted with one R²¹ group, R⁹ is heteroaryl substituted with one R²¹ group, said R⁹ is imidazolyl, said R²¹ group on said R¹⁰ moiety is —OR¹⁵, said R¹⁵ is alkyl, said R²¹ group on said R⁹ moiety is alkyl, and said R¹ is selected from the group consisting of: 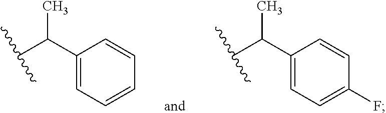
or
(g) for the compound in (a), W is —S(O)₂, R⁸ is H, R¹⁰ is aryl substituted with one R²¹ group, R⁹ is heteroaryl substituted with one R²¹ group, said R⁹ is imidazolyl, wherein said R²¹ group on said R¹⁰ moiety is ‥OR¹⁵, said R¹⁵ is alkyl, said R²¹ group on said R⁹ moiety is alkyl, and said R¹ is selected from the group consisting of:

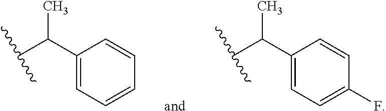

74.-83. (canceled)

84. The compound of claim 49 wherein:
(a) for the compound in (b) in claim 49, W is —S(O)₂, R⁸ is H, R¹⁰ is aryl substituted with one R²¹ group, R⁹ is heteroaryl substituted with one R²¹ group, and R¹ is alkyl substituted with one R²¹ group; or
(b) for the compound in (b) in claim 49, W is —S(O)₂, R⁸ is H, R¹⁰ is aryl substituted with one R²¹ group, R⁹ is heteroaryl substituted with one R²¹ group, R¹ is alkyl substituted with one R²¹ group said R²¹ group on said R¹⁰ moiety is —OR¹⁵, said R²¹ group on said R⁹ moiety is alkyl, and said R²¹ group on said R¹ group is aryl; or
(c) for the compound in (b) in claim 49, W is —S(O)₂, R⁸ is H, R¹⁰ is aryl substituted with one R²¹ group, R⁹ is heteroaryl substituted with one R²¹ group, R¹ is alkyl substituted with one R²¹ group, said R²¹ group on said R¹⁰ moiety is —OR¹⁵, said R²¹ group on said R⁹ moiety is alkyl, and said R²¹ group on said R¹ group is aryl substituted with one R²² group; or
(d) for the compound in (b) in claim 49, W is —S(O)₂, R⁸ is H, R¹⁰ is aryl substituted with one R²¹ group, R⁹ is heteroaryl substituted with one R²¹ group, R¹ is alkyl substituted with one R²¹ group said R²¹ group on said R¹⁰ moiety is —OR¹⁵, said R¹⁵ is alkyl, said R²¹ group on said R⁹ moiety is alkyl, and said R²¹ group on said R¹ group is aryl; or
(e) for the compound in (b) in claim 49, W is —S(O)₂, R⁸ is H, R¹⁰ is aryl substituted with one R²¹ group, R⁹ is heteroaryl substituted with one R²¹ group, R¹ is alkyl substituted with one R²¹ group, said R²¹ group on said R¹⁰ moiety is —OR¹⁵, said R¹⁵ is alkyl, said R²¹ group on said R⁹ moiety is alkyl, and said R²¹ group on said R¹ group is aryl substituted with one R²² group; or
(f) for the compound in (b) in claim 49, W is —S(O)₂, R⁸ is H, R¹⁰ is aryl substituted with one R²¹ group, R⁹ is heteroaryl substituted with one R²¹ group, said heteroaryl moiety is imidazolyl, R¹ is alkyl substituted with one R²¹ group, said R²¹ group on said R¹⁰ moiety is —OR¹⁵, said R¹⁵ is alkyl, said R²¹ group on said R⁹ moiety is alkyl, and said R²¹ group on said R¹ group is aryl; or
(g) for the compound in (b) in claim 49, W is —S(O)₂, R⁸ is H, R¹⁰ is aryl substituted with one R²¹ group, R⁹ is heteroaryl substituted with one R²¹ group, said heteroaryl moiety is imidazolyl, R¹ is alkyl substituted with one R²¹ group said R²¹ group on said R¹⁰ moiety is —OR¹⁵, said R¹⁵ is alkyl, said R²¹ group on said R⁹ moiety is alkyl, and said R²¹ group on said R¹ group is aryl substituted with one R²² group; or
(h) for the compound in (b) in claim 49, W is —S(O)₂, R⁸ is H, R¹⁰ is aryl substituted with one R²¹ group, R⁹ is heteroaryl substituted with one R²¹ group, said heteroaryl moiety is imidazolyl, said R²¹ group on said R¹⁰ moiety is —OR¹⁵, said R¹⁵ is alkyl, said R²¹ group on said R⁹ moiety is alkyl, and R¹ is: 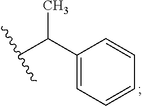
or
(i) for the compound in (b) in claim 49, W is —S(O)₂, R⁸ is H, R¹⁰ is aryl substituted with one R²¹ group, R⁹ is heteroaryl substituted with one R²¹ group, said heteroaryl moiety is imidazolyl, said R²¹ group on said R¹⁰ moiety is —OR¹⁵, said R¹⁵ is alkyl, said R²¹ group on said R⁹ moiety is alkyl, and R¹ is:

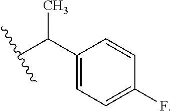

85.-94. (canceled)

95. The compound of claim 1:
(a) selected from the group consisting of: (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1), (IF.2), (IG), (IG.1), (IG.2), (IH), (IJ), (IK), (IL) and (IM); or
(b) selected from the group consisting of: (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IF), (IF.1), (IF.2), (IG), (IG.1), (IG.2), (IH), (IJ), (IK), (IL) and (IM), wherein W is —S(O)₂—; or
(c) selected from the group consisting of: (IA), (IA.1), (IB), (IB.1), (IC), (IC.1), (IC.2), (ID), (ID.1), (ID.2), (ID.3), (ID.4), (ID.5), (ID.6), (ID.7), (ID.8), (IE), (IE.1), (IE.2), (IF), (IF.1, (IF.2, (IG), (IG.1), (IG.2), (IH), (IJ), (IK), (IL) and (IM), where W is —S(O)—.

96.-97. (canceled)

98. A compound selected from the group consisting of:

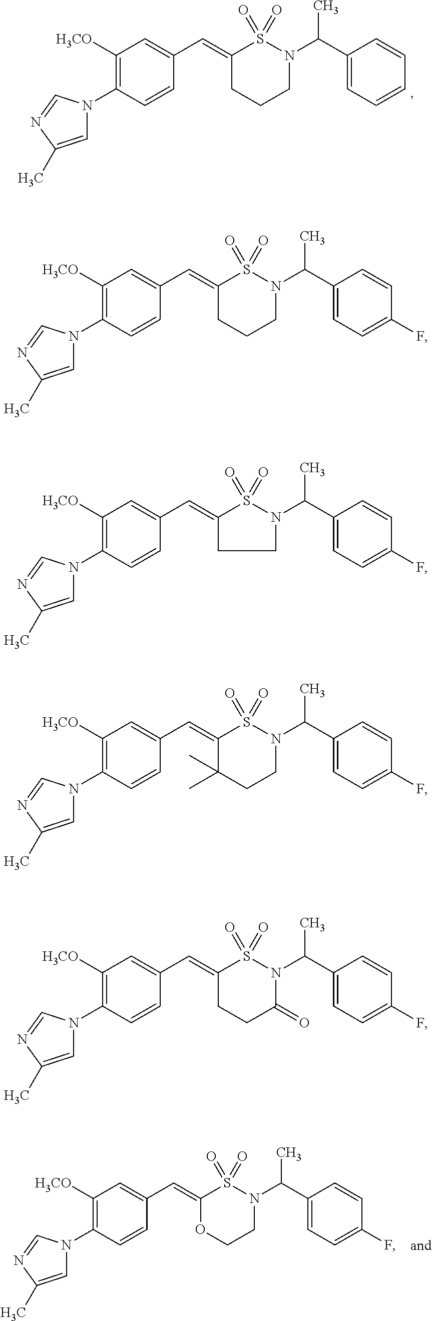

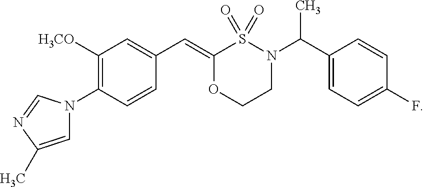

99. The compound of claim 1 selected from the group consisting of:

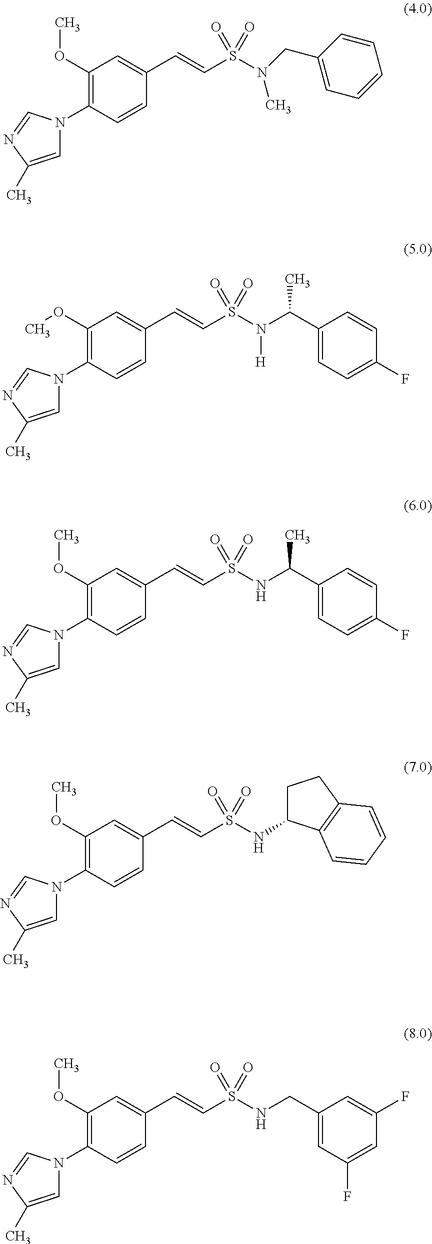

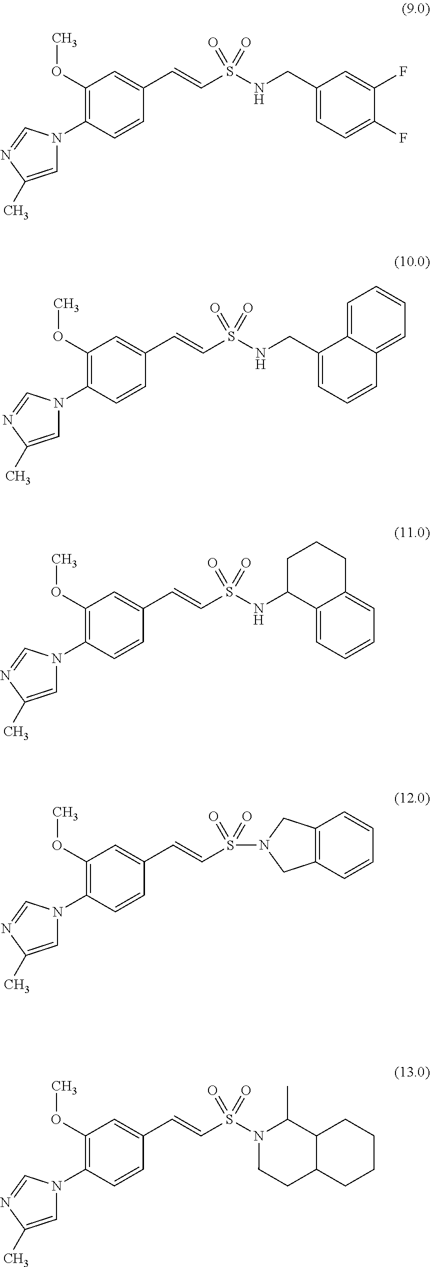

100.-116. (canceled)

117. A pharmaceutical composition:
(a) comprising a therapeutically effective amount of one or more compounds of claim 1, and a pharmaceutically acceptable carrier; or
(b) comprising an effective amount of one or more compounds of claim 1, and an effective amount of one or more other pharmaceutically active ingredients, and a pharmaceutically acceptable carrier, said other pharmaceutically active ingredients are selected form the group consisting of: (a) drugs useful for the treatment of Alzheimer's disease, (b) drugs useful for inhibiting the deposition of amyloid protein in, on or around neurological tissue, (c) drugs useful for treating neurodegenerative diseases, and (d) drugs useful for inhibiting gamma-secretase; or
(c) comprising an effective amount of one or more compounds of claim 1, and an effective amount of one or more other pharmaceutically active ingredients, and a pharmaceutically acceptable carrier, said other pharmaceutically active ingredients are selected from the group consisting of: BACE inhibitors; muscarinic antagonists; cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon; Cognex; Tau kinase inhibitors; anti-Abeta vaccine; APP ligands; agents that upregulate insulin cholesterol lowering agents; cholesterol absorption inhibitors; fibrates; LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin; or
(d) comprising a therapeutically effective amount of one or more compounds of claim 1, a pharmaceutical acceptable carrier, and a therapeutically effective amount of one or compounds selected from the group consisting of: cholinesterase inhibitors, Aβ antibody inhibitors, gamma secretase inhibitors and beta secretase inhibitors; or
(e) comprising a therapeutically effective amount of one or more compounds of claim 1, a pharmaceutically acceptable carrier, and a therapeutically effective amount of one or compounds selected from the group consisting of: donepezil hydrochloride, Aβ antibody inhibitors, gamma secretase inhibitors and beta secretase inhibitors; or
(f) a therapeutically effective amount of one or more compounds of claim 1, and a pharmaceutically acceptable carrier, and an effective amount of one or more BACE inhibitors.

118.-130. (canceled)

131. A method of treating Alzheimers disease:
(a) comprising administering a therapeutically effective amount of at least one compound of claim 1 to a patient in need of such treatment; or
(b) comprising administering an effective amount of one or more compounds of claim 1 in combination with an effective amount of: (1) one or more cholinesterase inhibitors; or (2) donepezil hydrochloride; or (3) one or more compounds selected from the group consisting of Aβ antibody inhibitors, gamma secretase inhibitors and beta secretase inhibitors; or (4) one or more BACE inhibitors; or (5) Exelon; or (6) Cognex; or (7) a Tau kinase inhibitor; or (8) a Tau kinase inhibitor selected from the group consisting of: GSK3beta inhibitors, cdk5 inhibitors, and ERK inhibitors; or (9) one anti-Abeta vaccination; or (10) one or more APP ligands; or (11) one or more agents that upregulate insulin degrading enzyme and/or neprilysin; or (12) one or more cholesterol lowering agents; or (13) one or more cholesterol lowering agents selected from the group consisting of statins and cholesterol absorption inhibitors; or (14) one or more cholesterol lowering agents selected from the group consisting of: Atorvastatin, Fluvastatin, Lovastatin, Mevastatin, Pitavastatin, Pravastatin, Rosuvastatin, Simvastatin, and Ezetimibe; or (15) one or more fibrates; or (16) one or more fibrates selected from the group consisting of: clofibrate, Clofibride, Etofibrate, and Aluminium Clofibrate; or (17) one or more LXR agonists; or (18) one or more LRP mimics; or (19) one or more 5-HT6 receptor antagonists; or (20) one or more nicotinic receptor agonists; or (21) one or more H3 receptor antagonists; or (22) one or more histone deacetylase inhibitors; or (23) one or more hsp90 inhibitors; or (24) one or more m1 muscarinic receptor agonists; or (25) one or more 5-HT6 receptor antagonists, mGluR1, mGluR5, or positive allosteric modulators or agonists; or (26) one or more mGluR2/3 antagonists; or (27) one or more anti-inflammatory agents that can reduce neuroinflammation; or (28) one or more Prostaglandin EP2 receptor antagonists; or (29) one or more PAI-1 inhibitors; or (30) one or more agents that can induce Abeta efflux; or (31) gelsolin.

132.-136. (canceled)

137. A method of:
(1) modulating gamma-secretase comprising administering an effective amount of one or more compounds of claim 1 to a patient in need of such treatment; or
(2) treating one or more neurodegenerative diseases comprising administering an effective amount of one or more compounds of claim 1 to a patient in need of treatment; or
(3) inhibiting the deposition of amyloid protein in, on or around neurological tissue, comprising administering an effective amount of one or more compounds of claim 1 to a patient in need of treatment; or
(4) treating mild cognitive impairment comprising administering an effective amount of one or more compounds of claim 1 to a patient in need of treatment
(5) treating glaucoma comprising administering an effective amount of one or more compounds of claim 1 to a patient in need of treatment; or
(6) treating cerebral amyloid angiopathy comprising administering an effective amount of one or more compounds of claim 1 to a patient in need of treatment; or
(7) treating stroke comprising administering an effective amount of one or more compounds of claim 1 to a patient in need of treatment; or
(8) treating dementia comprising administering an effective amount of one or more compounds of claim 1 to a patient in need of treatment, or
(9) treating microgliosis comprising administering an effective amount of one or more compounds of claim 1 to a patient in need of treatment; or
(10 treating brain inflammation comprising administering an effective amount of one or more compounds of claim 1 to a patient in need of treatment; or
(11) treating olfactory function loss comprising administering an effective amount of one or more compounds of claim 1 to a patient in need of treatment; or
(12) treating a central nervous system disorder comprising administering a therapeutically effective amount of at least one compound of claim 1 to a patient in need of such treatment; or
(13) treating Downs syndrome comprising administering a therapeutically effective amount of at least one compound of claim 1 to a patient in need of such treatment; or
(14) treating down's syndrome comprising administering an effective amount of one or more compounds of claim 1, in combination with an effective amount of one or more cholinesterase inhibitors, to a patient in need of treatment; or
(15) treating down's syndrome comprising administering an effective amount of one or more compounds of claim 1, in combination with an effective amount of donepezil hydrochloride, to a patient in need of treatment.

138.-142. (canceled)

143. A kit comprising, in separate containers, in a single package, pharmaceutical compositions for use in combination, wherein one container comprises an effective amount of a compound of claim 1 in a pharmaceutically acceptable carrier, and another container comprises an effective amount of another pharmaceutically active ingredient, the combined quantities of the compound of claim 1 and the other pharmaceutically active ingredient being effective to: (a) treat Alzheimer's disease, or (b) inhibit the deposition of amyloid protein in, on or around neurological tissue, or (c) treat neurodegenerative diseases, or (d) modulate the activity of gamma-secretase.

144.-145. (canceled)

146. A compound of the formula: 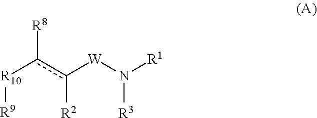
or the pharmaceutically acceptable salts thereof, wherein:
R¹, R², R³, R⁸, R⁹, R¹⁰, and W are independently selected;
W is selected from the group consisting of; —S(O)—, and —S(O)₂—;
R¹ is selected from the group consisting of H, alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedheterocycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl- R¹ groups is optionally substituted with 1-5 independently selected R²¹ groups;
R² and R³ are each independently selected from the group consisting of H, alkyl-, alkenyl-, alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl-, and heterocyclyalkyl-; wherein each of said alkyl-, alkenyl- and alkynyl-, aryl-, arylalkyl-, alkylaryl-, cycloalkyl-, cycloalkenyl, cycloalkylalkyl-, cycloalkenyl-, heteroaryl-, heteroarylalkyl-, heterocyclyl-, heterocyclenyl- and heterocyclyalkyl- R¹ groups is optionally substituted with 1-5 independently selected R²¹ groups; or
R² and R³ taken together, along with the atoms to which they are bound, form a ring selected from the group consisting of:
(a) a 5 to 6 membered heterocycloalkyl ring, said heterocycloalkyl ring optionally comprising, in addition to W and in addition to the N adjacent to W, at least one other heteroatom independently selected from the group consisting of: —O—, —NR¹⁴—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one other heteroatom independently selected from the group consisting of: —O—, —NR¹⁴—, —S(O)—, —S(O)₂, and —C(O)—, and
wherein said ring is optionally substituted with 1-5 independently selected R²¹ groups; or
R² and R³ taken together along with the atoms to which they are bound, and R¹ and R³ are taken together along with the atoms to which they are bound, form the fused ring moiety: 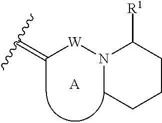
wherein Ring A is a ring selected from the group consisting of:
(a) a 5 to 6 membered heterocycloalkyl ring, said heterocycloalkyl ring optionally comprising, in addition to W and in addition to the N adjacent to W, at least one other heteroatom independently selected from the group consisting of: —O—, —NR¹⁴—, —S(O)—, —S(O)₂, and —C(O)—, and
(b) a 5 to 6 membered heterocycloalkenyl ring, said heterocycloalkenyl ring optionally comprising, in addition to W and in addition to the N adjacent to W, at least one other heteroatom independently selected from the group consisting of: —O—, —NR¹⁴—, —S(O)—, —S(O)₂, and —C(O)—, and
wherein said fused ring moiety is optionally substituted with 1-5 independently selected R²¹ groups; or
R¹ and R³ taken together with the atoms to which they are bound form a fused benzoheterocycloalkyl ring, and wherein said fused ring is optionally substituted with 1-5 independently selected R²¹ groups,
R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl-; wherein each of said R⁸ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl- and heterocyclyalkyl- is optionally substituted with 1-3 independently selected R²¹ groups;
R⁹ is selected from the group consisting of: alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, and heterocyclyalkyl-, wherein each of said R⁹ alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl- and heterocyclyalkyl- is optionally substituted with 1-3 independently selected R²¹ groups;
R¹⁰ is selected from the group consisting of: a bond, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl-, heterocyclyalkyl-, heterocyclyalkenyl-, 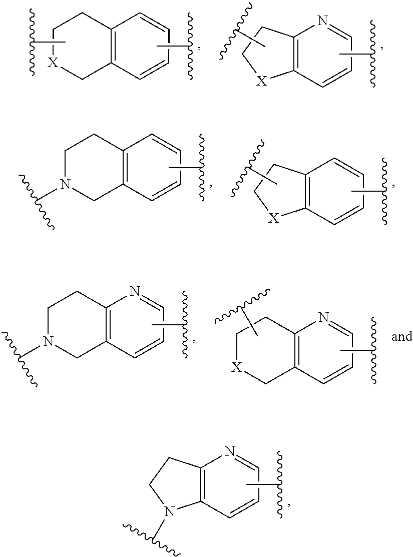
wherein X is selected from the group consisting of: O, —N(R¹⁴)— or —S—; and
wherein each of said R¹⁰ moieties is optionally substituted with 1-3 independently selected R²¹ groups;
R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);
R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;
Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or
two R¹⁸ moieties on adjacent carbons can be linked together to form a 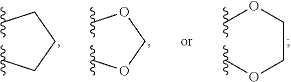
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl;
Each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —ON, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups; and
Each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶—CH₂—N(R¹⁵)C(O)OR¹⁶—N₃, ═NOR¹⁵, —NO₂, —S(O)R¹⁵ and —S(O)₂R¹⁵.

147. The compound of claim 146 wherein the optional bond is absent and the compound of formula (A) is [chemical structure]
